(19) United States
(12) Patent Application Publication
(10) Pub. No.: US 2021/0113378 A1
(43) Pub. Date: Apr. 22, 2021

(54) APPARATUS AND METHOD FOR CATARACT EXTRACTION

(71) Applicant: NEW WORLD MEDICAL, INC., Rancho Cucamonga, CA (US)

(72) Inventors: Malik KAHOOK, Denver, CO (US); Suhail ABDULLAH, Fontana, CA (US); Khalid MANSOUR, Corona, CA (US); Eric PORTEOUS, Corona, CA (US); Joey TRAN, Ontario, CA (US); Patrick CHEN, Hacienda Heights, CA (US); Vijay R. BALAN, Rancho Cucamonga, CA (US); Cristos IFANTIDES, Denver, CO (US)

(21) Appl. No.: 17/052,127

(22) PCT Filed: Apr. 25, 2019
(86) PCT No.: PCT/US2019/029206

§ 371 Date: Oct. 30, 2020

Related U.S. Application Data
(60) Provisional application No. 62/666,640, filed on May 3, 2018.

(51) Int. Cl. A61F 9/007; A61B 17/3205

(52) U.S. Cl. CPC A61F 9/00763; A61B 2017/32006; A61B 17/32056

(57) ABSTRACT

Apparatus and methods for cataract extraction are disclosed. An extraction device may include a delivery shaft having a lumen and a distal end. The lumen may include a first channel and a second channel diverging at the distal end. A wire may form a loop and extend from the first channel and the second channel. The wire may be movable within the lumen.

### CROSS-REFERENCE TO RELATED APPLICATIONS

This application is the national stage of International Patent Application No. PCT/US2019/029206, entitled “APPARATUS AND METHOD FOR CATARACT EXTRACTION,” filed Apr. 25, 2019, which claims the benefit of U.S. Provisional Application No. 62/666,640, entitled “APPARATUS AND METHOD FOR CATARACT EXTRACTION,” filed May 3, 2018, the entirety of which is incorporated herein by reference.

### TECHNICAL FIELD

The present disclosure generally relates to surgical devices and, in particular, relates to cataract extraction devices.

### BACKGROUND

Cataracts often cause loss of vision by clouding the lens of the eye. Cataract surgery is often performed, in which the lens is removed from the eye and replaced with a synthetic lens. However, if care is not taken, it can be difficult to remove the lens.

### BRIEF DESCRIPTION OF THE DRAWINGS

The accompanying drawings, which are included to provide further understanding of the subject technology and are incorporated in and constitute a part of this description, illustrate aspects of the subject technology and, together with the specification, serve to explain principles of the subject technology.

FIG. 1A shows a perspective view of an extraction device, in accordance with one or more embodiments of the present disclosure.

FIG. 1B shows a perspective view of the extraction device of FIG. 1A, in accordance with one or more embodiments of the present disclosure.

FIG. 1C shows a perspective view of the extraction device of FIG. 1A, in accordance with one or more embodiments of the present disclosure.

FIG. 1D shows a perspective view of the extraction device of FIG. 1A, in accordance with one or more embodiments of the present disclosure.

FIG. 1E shows a perspective view of the extraction device of FIG. 1A, in accordance with one or more embodiments of the present disclosure.

FIG. 1F shows a perspective view of the extraction device of FIG. 1A, in accordance with one or more embodiments of the present disclosure.

FIG. 1G shows a perspective view of the extraction device of FIG. 1A, in accordance with one or more embodiments of the present disclosure.

FIG. 2A shows a perspective view of an extraction device, in accordance with one or more embodiments of the present disclosure.

FIG. 2B shows a perspective view of the extraction device of FIG. 2A, in accordance with one or more embodiments of the present disclosure.

FIG. 3A shows a perspective view of an extraction device, in accordance with one or more embodiments of the present disclosure.

FIG. 3B shows a perspective view of an extraction device, in accordance with one or more embodiments of the present disclosure.

FIG. 4 shows a perspective view of an extraction device, in accordance with one or more embodiments of the present disclosure.

FIG. 5A shows a perspective view of an interface tool, in accordance with one or more embodiments of the present disclosure.

FIG. 5B shows a perspective view of the interface tool of FIG. 5A, in accordance with one or more embodiments of the present disclosure.

FIG. 6A shows a perspective view of an extraction device, in accordance with one or more embodiments of the present disclosure.

FIG. 6B shows a perspective view of the extraction device of FIG. 6A, in accordance with one or more embodiments of the present disclosure.

FIG. 7 shows a perspective view of an extraction device, in accordance with one or more embodiments of the present disclosure.

FIG. 8 shows a perspective view of an extraction device, in accordance with one or more embodiments of the present disclosure.

FIG. 9 shows a perspective view of an extraction device, in accordance with one or more embodiments of the present disclosure.

FIG. 10A shows a perspective view of an extraction device, in accordance with one or more embodiments of the present disclosure.

FIG. 10B shows a perspective view the extraction device of FIG. 10A, in accordance with one or more embodiments of the present disclosure.

FIG. 10C shows a perspective view of the extraction device of FIG. 10A, in accordance with one or more embodiments of the present disclosure.

FIG. 10D shows an end view of the extraction device of FIG. 10A, in accordance with one or more embodiments of the present disclosure.

FIG. 10E shows a perspective view of the extraction device of FIG. 10A, in accordance with one or more embodiments of the present disclosure.

FIG. 11A shows a perspective view of an extraction device, in accordance with one or more embodiments of the present disclosure.

FIG. 11B shows a perspective view the extraction device of FIG. 11A, in accordance with one or more embodiments of the present disclosure.

FIG. 12A shows a perspective view of an extraction device, in accordance with one or more embodiments of the present disclosure.

FIG. 12B shows a perspective view the extraction device of FIG. 12A, in accordance with one or more embodiments of the present disclosure.

FIG. 13A shows a perspective view of an extraction device, in accordance with one or more embodiments of the present disclosure.

FIG. 13B shows a perspective view the extraction device of FIG. 13A, in accordance with one or more embodiments of the present disclosure.

FIG. 14A shows a perspective view of an extraction device, in accordance with one or more embodiments of the present disclosure.

FIG. 14B shows a perspective view the extraction device of FIG. 14A, in accordance with one or more embodiments of the present disclosure.

FIG. 14C shows a perspective view the extraction device of FIG. 14A, in accordance with one or more embodiments of the present disclosure.

FIG. 15A shows a perspective view of an extraction device, in accordance with one or more embodiments of the present disclosure.

FIG. 15B shows a cross-sectional view the extraction device of FIG. 15A, in accordance with one or more embodiments of the present disclosure.

FIG. 16A shows a side view of an extraction device, in accordance with one or more embodiments of the present disclosure.

FIG. 16B shows a cross-sectional side view the extraction device of FIG. 16A, in accordance with one or more embodiments of the present disclosure.

FIG. 16C shows a top view the extraction device of FIG. 16A, in accordance with one or more embodiments of the present disclosure.

FIG. 16D shows a top view the extraction device of FIG. 16A, in accordance with one or more embodiments of the present disclosure.

FIG. 16E shows a top view the extraction device of FIG. 16A, in accordance with one or more embodiments of the present disclosure.

FIG. 17A shows a front view of an extraction device, in accordance with one or more embodiments of the present disclosure.

FIG. 17B shows a side angle view the extraction device of FIG. 17A, in accordance with one or more embodiments of the present disclosure.

FIG. 18 shows a perspective view of an extraction device, in accordance with one or more embodiments of the present disclosure.

FIG. 19 shows a perspective view of a lid structure for the extraction device of FIG. 18, in accordance with one or more embodiments of the present disclosure.

FIG. 20 shows a cross-sectional view of a portion of the extraction device of FIG. 18, in accordance with one or more embodiments of the present disclosure.

FIG. 21 shows a perspective view of a frame structure for the extraction device of FIG. 18, in accordance with one or more embodiments of the present disclosure.

FIG. 22 shows a cross-sectional perspective view of a portion of the extraction device of FIG. 18, in accordance with one or more embodiments of the present disclosure.

FIG. 23 shows a cross-sectional perspective view of a portion of the extraction device of FIG. 18, in accordance with one or more embodiments of the present disclosure.

FIG. 24 shows a perspective view of a portion of an extraction device, in accordance with one or more embodiments of the present disclosure.

FIG. 25 shows a side view of a portion of the extraction device of FIG. 24, in accordance with one or more embodiments of the present disclosure.

FIG. 26 shows a perspective view of a portion of the extraction device of FIG. 24 including a portion of a handle, in accordance with one or more embodiments of the present disclosure.

FIG. 27 shows a perspective the extraction device of FIG. 24, in accordance with one or more embodiments of the present disclosure.

FIG. 28 shows a perspective view of a portion of an extraction device having three wire loops, in accordance with one or more embodiments of the present disclosure.

FIG. 29 shows a perspective view of the portion of the extraction device of FIG. 28 with the wire loops in a rotated configuration, in accordance with one or more embodiments of the present disclosure.

FIG. 30 shows a perspective view of a portion of an extraction device having two interconnected wire loops in a flattened configuration, in accordance with one or more embodiments of the present disclosure.

FIG. 31 shows a perspective view of the portion of the extraction device of FIG. 30 with the interconnected wire loops in a rotated configuration, in accordance with one or more embodiments of the present disclosure.

FIG. 32 shows a perspective view of a portion of another extraction device having two interconnected wire loops in a flattened configuration, in accordance with one or more embodiments of the present disclosure.

FIG. 33 shows a perspective view of the portion of the extraction device of FIG. 32 with the interconnected wire loops in a separated configuration, in accordance with one or more embodiments of the present disclosure.

FIG. 34 shows a perspective view of a portion of another extraction device having three interconnected wire loops in a flattened configuration, in accordance with one or more embodiments of the present disclosure.

FIG. 35 shows a side view of the portion of the extraction device of FIG. 34, in accordance with one or more embodiments of the present disclosure.

FIG. 36 shows a face-on view of the extraction device of FIG. 30, in accordance with one or more embodiments of the present disclosure.

FIG. 37 shows a face-on view of the extraction device of FIG. 31, in accordance with one or more embodiments of the present disclosure.

FIG. 38 shows a face-on view of the extraction device of FIG. 32, in accordance with one or more embodiments of the present disclosure.

FIG. 39 shows a face-on view of the extraction device of FIG. 33, in accordance with one or more embodiments of the present disclosure.

FIG. 40 shows a face-on view of the extraction device of FIG. 34, in accordance with one or more embodiments of the present disclosure.

FIG. 41 shows a perspective view of a portion of an extraction device having wire loops, a stent, and an encapsulation bag, in accordance with one or more embodiments of the present disclosure.

FIG. 42 shows a side view of the extraction device of FIG. 41, in accordance with one or more embodiments of the present disclosure.

FIG. 43 shows a perspective view of the wire loops of the extraction device of FIG. 41, in accordance with one or more embodiments of the present disclosure.

FIG. 44 shows a perspective view of a lens cut by the wire loops of FIG. 43, in accordance with one or more embodiments of the present disclosure.

FIG. 45 shows a perspective view of the stent of the extraction device of FIG. 41, in accordance with one or more embodiments of the present disclosure.

FIG. 46 shows a perspective view of a lens cubed by the stent of FIG. 45, in accordance with one or more embodiments of the present disclosure.

FIG. 47 shows a perspective view of the encapsulation bag of the extraction device of FIG. 41, in accordance with one or more embodiments of the present disclosure.

FIG. 48 shows a perspective view of the encapsulation bag of the extraction device of FIG. 41 in a collapsed configuration, in accordance with one or more embodiments of the present disclosure.

FIG. 49 shows a cross-sectional view of the extraction device of FIG. 41, in accordance with one or more embodiments of the present disclosure.

FIG. 50 shows a perspective view of the extraction device of FIG. 41 during cutting of the lens by the wire loops, in accordance with one or more embodiments of the present disclosure.

FIG. 51 shows a perspective view of the extraction device of FIG. 41 after cutting of the lens by the wire loops, in accordance with one or more embodiments of the present disclosure.

FIG. 52 shows a perspective view of the extraction device of FIG. 41 after cubing of the lens by the stent and after collapsing of the encapsulation bag, in accordance with one or more embodiments of the present disclosure.

FIG. 53A shows a perspective view of an extraction device, in accordance with one or more embodiments of the present disclosure.

FIG. 53B shows a top view of a wire that can be used in the extraction device from FIG. 53A, in accordance with one or more embodiments of the present disclosure.

FIG. 53C shows a top view of an extraction device using the wire from FIG. 53B, in accordance with one or more embodiments of the present disclosure.

FIGS. 54A-54C show side views of the extraction device from FIG. 53A interacting with a specimen, in accordance with one or more embodiments of the present disclosure.

FIG. 55A shows a perspective view of the extraction device from FIG. 53A including a handle, in accordance with one or more embodiments of the present disclosure.

FIGS. 55B-55C show cut-away views of the extraction device from FIG. 53A including a handle, in accordance with one or more embodiments of the present disclosure.

FIG. 55D shows a perspective view of the extraction device from FIG. 53A including a handle implemented with a push button, in accordance with one or more embodiments of the present disclosure.

FIGS. 56A-56C show various views of the extraction device from FIG. 53A including an articulating joint, in accordance with one or more embodiments of the present disclosure.

FIGS. 56D-56F show cutaway views of the extraction device from FIG. 53A including an articulating joint, in accordance with one or more embodiments of the present disclosure.

FIG. 57 shows a top view of the extraction device from FIG. 53A, in accordance with one or more embodiments of the present disclosure.

FIGS. 58A-58C shows top views of the extraction device from FIG. 53A, implemented with various cutting faces at the delivery shaft distal end, in accordance with one or more embodiments of the present disclosure.

FIG. 58D shows a perspective view of the extraction device from FIG. 53A, implemented with a cutting face at the delivery shaft distal end, in accordance with one or more embodiments of the present disclosure.

FIG. 59 shows a perspective view of the extraction device from FIG. 53A implemented with a sleeve, in accordance with one or more embodiments of the present disclosure.

FIG. 60 shows a top view of the extraction device from FIG. 53A interacting with a lens and capsular bag, in accordance with one or more embodiments of the present disclosure.

### DETAILED DESCRIPTION

In the following detailed description, specific details are set forth to provide an understanding of the subject technology. It will be apparent, however, to one ordinarily skilled in the art that the subject technology may be practiced without some of these specific details. In other instances, well-known structures and techniques have not been shown in detail so as not to obscure the subject technology.

A handheld device, sometimes referred to herein as a cataract extraction device or an extraction device, is provided that can be used to break the lens of the eye of a patient into a desired number of pieces and remove the resulting pieces from the eye through an incision. For example, after a hydrodissection of the lens has been performed to displace the lens from its capsule, the extraction device can be moved into the eye through a corneal incision and be utilized to encapsulate or partially surround the lens and remove it in a controlled manner in one or more pieces. As another example, the extraction device can be utilized to encircle and cut the lens into smaller pieces while the lens is within the lens capsule. A second device can be used to flush the eye of any lens remnants.

The extraction device may include additional mechanisms or components to control the motion of lens elements out of the anterior chamber of the eye. The devices can be provided together as a kit. In some embodiments, the extraction device can contain at least a part of the lens during and/or after hydrodissection. If the lens is undesirably displaced during hydrodissection, the device can be used to recapture the lens or a portion thereof.

In accordance with various embodiments described in further detail below, an extraction device may be provided with a delivery shaft and a manipulator having one or more components or features that can be extended from a lumen of the delivery shaft to cut, grab, encapsulate, and/or otherwise manipulate the lens of a patient's eye for extraction.

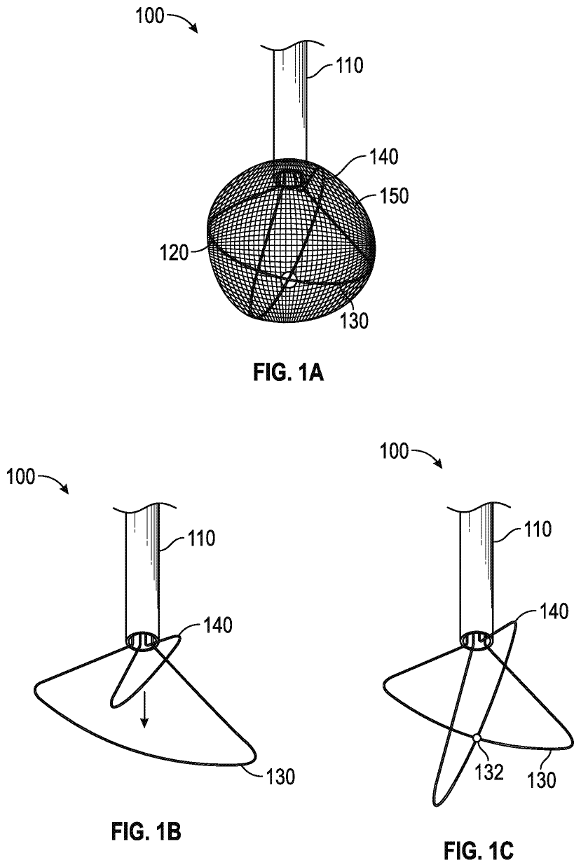

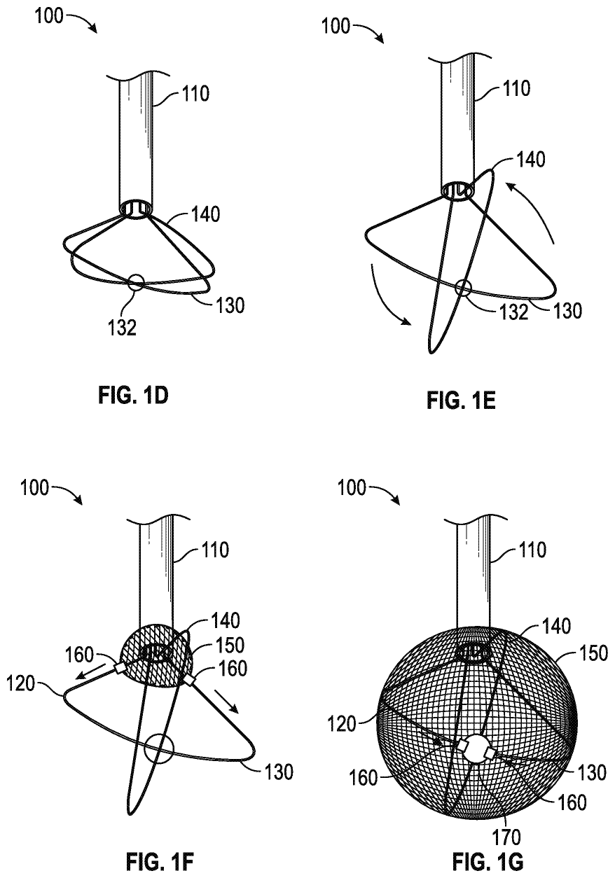

According to some embodiments, for example as shown in FIG. 1A, an extraction device 100 can include a delivery shaft 110 having a lumen and a distal end. A cover 150 can encompass an outer cross-sectional dimension of a capture region 120 defined, at least in part, by each of (i) the first wire 130 and (ii) the second wire 140, as described further herein. The wires can be textured and/or contain features that allow a lens to be grasped, to help break up the lens, and to grip the lens while it is being moved.

The first wire 130 can form a first arc portion (e.g., loop) distal to the distal end of the delivery shaft 110, and the second wire 140 can form a second arc portion (e.g., loop) distal to the distal end of the delivery shaft 110. Each of the first wire 130 and the second wire 140 can be separately retractable relative to the delivery shaft 110. As shown in FIGS. 1B and 1C, the first wire 130 can be deployed from the lumen of the delivery shaft 110 before the second wire 140 is deployed. The second wire 140 can be deployed until it reaches a distalmost end 132 of the first wire 130. The distalmost end of the second wire 140 can remain proximal to the distalmost end 132 of the first wire 130. Additionally, the second wire 140 can be retracted into the lumen of the delivery shaft 110 before the first wire 130 is retracted. According to some embodiments, the separate wires can be connected to each other and can be advanced and/or retracted at the same time. According to some embodiments, the wires can also rotate in the same direction and/or in multiple directions prior to and/or during advancement and/or retraction. According to some embodiments, one or more wires can be contained within another wire. For instance, a larger diameter wire can have a groove for receiving a smaller wire. A third wire is connected to a cover, such as a net or bag. Once the lens is encapsulated in the cover, the smaller second wire is actuated and moves along the groove of the first wire to dissect the lens.

The first wire 130 and the second wire 140 can be rings of metal (e.g., nitinol), plastic, and/or a shape memory material. The capture region 120 may also include other materials, such as a suture or polymer that acts to encircle the lens once deployed from the lumen. The rings can be closed by retraction to cut a lens of a patient into smaller pieces. Alternatively, or in combination, other mechanisms could be employed to dissect the lens, including scissors, forceps, or blades. Such tools can dissect the lens as the lens is held in position or move (e.g., rotate) the lens against a mechanism to cut the lens. Alternatively, or in combination, any number of wires can be provided. For example, wires in addition to the first wire 130 and the second wire 140 can be provided in a nested formation, such that each wire is deployed within a region bounded by an adjacent wire.

According to some embodiments, for example as shown in FIGS. 1D and 1E, the first wire 130 and the second wire 140 can be rotated relative to each other before, during, or after deployment from the delivery shaft 110. As shown in FIG. 1D, the first wire 130 and the second wire 140 can be positioned to be substantially flat. As shown in FIG. 1E, the first wire 130 and the second wire 140 can be positioned to have an enlarged region of enclosure within the first wire 130 and the second wire 140. For example, the first wire 130 can reside within a plane that is substantially perpendicular to a plane within which the second wire 140 resides.

According to some embodiments, for example as shown in FIGS. 1F and 1G, the cover 150 can be moved about the capture region 120. One or more guides 160 can be moveable along the arc portion of the first wire 130 toward the distalmost end 132 of the first wire 130. The guides 160 can deploy the cover 150 about the capture region 120 as they move along the first wire 130 from a first position (see FIG. 1F) to a second position (see FIG. 1G), more distal than the first position. While in the second position, the cover 150 encompasses an outer cross-sectional dimension of each of (i) the first wire 130 and (ii) the second wire 140. The cover can extend such that an aperture 170 remains at the distalmost end 132 of the first wire 130. The guides 160 can partially or completely wrap around a wire 130, 140 and/or be able to travel along or within a groove of a wire 130, 140. The guides 160 can be actuated (e.g., advanced and/or retracted) by one or more push rods (not shown) that connect to the guides 160 and have a portion thereof that is accessible to the user. The push rods can extend through the delivery shaft 110. The push rods can be actuated based on mechanical activation (e.g., trigger or rack and pinion control) and/or electrical activation (e.g., motor).

The cover 150 can include polyethylene or similar compliant polymer. The cover 150 can include polyethylene and nitinol, PTFE and nitinol, a flexible polymer and/or nitinol, a substantially inflexible material and/or nitinol, a substantially inflexible material and/or a material that acts as a platform for the inflexible or flexible material membranes so that the membrane may be advanced efficiently over the capture region 120. Additional structures that can accompany a cover are discussed in greater detail herein. The cover 150 can be a molded piece of elastomeric material that is everted. The cover 150 can have a natural tendency to return to its original shape. A device that is controllable by a user can be brought into contact with the cover 150 to urge the cover 150 into a shape for which the cover 150 has a bias.

According to some embodiments, the capture region 120 can be introduced prior to the cover 150 and the cover 150 is deployed second over the capture region 120 which has already been positioned to hold the natural lens. According to some embodiments, the cover 150 can be introduced prior to the capture region 120 and the capture region 120 is deployed second over the cover 150 which has already been positioned to hold the natural lens. The cover 150 can be deployed to capture a lens before, during, or after the lens has been cut and/or dissected (e.g., hydrodissected from the lens capsule and/or divided into pieces) by a tool (e.g., the wires 130, 140 of the capture region 120). The cover 150 can be deployed from a proximal end of the capture region 120 and a distal direction or from a distal end of the capture region 120 in a proximal direction.

The capture region 120 and/or the cover 150 can be retracted in stages or in one movement into the lumen of the introducer. According to some embodiments, the material that forms the lumen used to introduce the capture region 120 and cover 150 is composed of flexible material that may expand when the capture region 120 and cover 150 are retracted and then returns to previous form.

According to some embodiments, the capture region 120 and cover 150 are maneuvered in and out of the eye with a screw type mechanism to control linear motion. According to some embodiments, the capture region 120 and cover 150 are maneuvered in and out of the eye with a hydraulic system to control linear motion. According to some embodiments, the capture region 120 and cover 150 are maneuvered in and out of the eye with a mechanical mechanism to control linear motion, such as a pneumatic control.

According to some embodiments, the capture region 120 and cover 150 are maneuvered in and out of the eye after the lens has been completely removed from the natural bag and positioned at the iris plane or above.

According to some embodiments, the capture region 120 and cover 150 are maneuvered to feed the lens into a cutting membrane that is present at the lumen of the device so as to cause further segmentation of the lens prior to retracting into the lumen. According to some embodiments, the capture region 120 and cover 150 are maneuvered to feed the lens using a twisting action so as to cause further segmentation of the lens prior to retracting into the lumen. According to some embodiments, a third structure is introduced that contains a second lumen that “punches a hole” within the body of the contained lens material so as to core out the central nucleus prior to being retracted into the lumen of the main device. According to some embodiments, the device lumen is connected to an inflow and outflow lumen that allows for fluid to enter and exit the eye and to maintain the anterior chamber. According to some embodiments, the device lumen is connected to an inflow and outflow lumen that allows for lens material to enter and exit the eye and to maintain the anterior chamber.

According to some embodiments, the cover 150 surrounds the lens which has been dissected out of the lens bag, and a cover 150 that contains a second lumen “punches a hole” within the body of the contained lens material so as to core out the central nucleus prior to being retracted into the lumen of the main device (no capture region 120 is used).

According to some embodiments, the extraction device is devoid of electronic components. According to some embodiments, the extraction device is operated using finger and hand controls. According to some embodiments, the extraction device is operated with foot pedal controls. According to some embodiments, the extraction device is operated using robotic controls.

According to some embodiments, the capture region 120 is used independently to segment the lens into smaller pieces and a second device, commonly known as phacoemulsification hand piece or irrigation/aspiration hand piece, is used to remove the segmented lens pieces.

According to some embodiments, the capture region 120 and/or the cover 150 are manufactured from materials that are substantially transparent to optical coherence tomography (OCT) imaging so as to not interfere or substantially not interfere with intraoperative OCT imaging. According to some embodiments, the structures of the extraction device are colored so the operator may easily differentiate between the parts.

According to some embodiments, the entire extraction device is disposable. According to some embodiments, some parts of the extraction device are disposable. For example, the capture region 120 and the cover 150 are designed so that removing them for cleaning will disallow further adjoining with the lumen shaft.

According to some embodiments, the extraction device, including capture region 120 and/or secondary component are designed to contain a plurality of holes that are 50 microns to 1 mm in size. The holes are designed to allow for segmentation of the lens into pieces equal to or smaller than 1 mm once the distal components are retracted back into the lumen. The segmented pieces, which remain in the anterior chamber, are then removed from the eye by an irrigation/aspiration extraction device.

According to some embodiments, the capture region 120 is discontinuous, with only partial surrounding of the lens prior to manipulation of the lens.

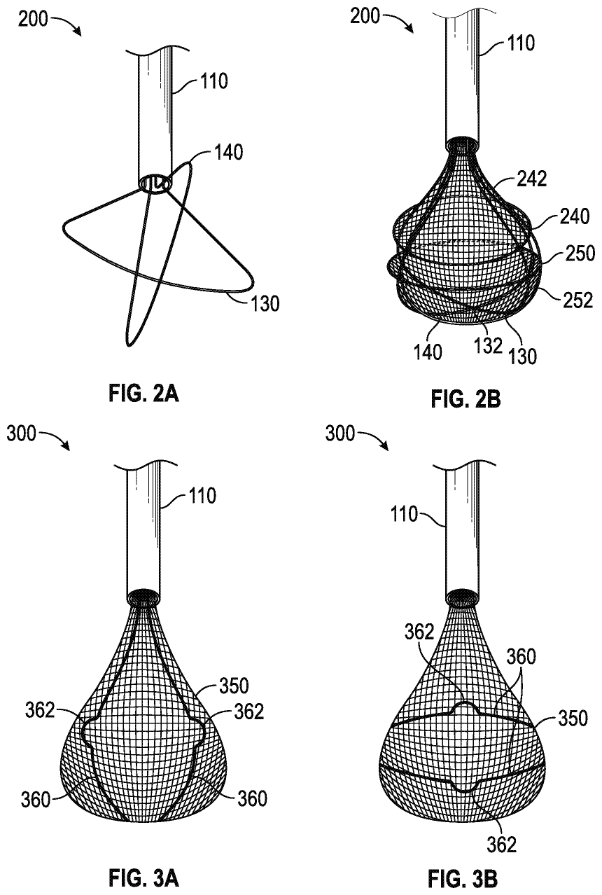

According to some embodiments, for example as shown in FIGS. 2A and 2B, an extraction device 200 can include a first ring 240 extending about the first wire 130 and the second wire 140 and a first cover 242 extending from the first ring 240 to the delivery shaft 110. The extraction device 200 can also include a second ring 250 extending about the first wire 130 and the second wire 140 and a second cover 252 extending from the second ring 250 to the distalmost end 132 of the first wire 130. The first cover 242 and the second cover 252 can enclose a space for capturing debris. The first ring 240 and the second ring 250 can be adjacent to each other or spaced apart. The first ring 240 and the second ring 250 can be actuated (e.g., advanced and/or retracted) by one or more push rods (not shown) that connect to the first ring 240 and the second ring 250 and have a portion thereof that is accessible to the user. The push rods can extend through the delivery shaft 110. The push rods can be actuated based on mechanical activation (e.g., trigger or rack and pinion control) and/or electrical activation (e.g., motor).

According to some embodiments, for example as shown in FIGS. 3A and 3B, an extraction device 300 can include a cover 350 that is advanceable and retractable over and around a target region. The extraction device 300 can further include one or more support bands 360 positioned on, within, or against the cover 350. The support bands 360 can provide a degree of rigidity to the cover 350, to urge the cover 350 to conform to a particular shape. The support bands 360 can be biased to a particular shape. For example, the support bands 360 can be a shape memory alloy. The support bands 360 can extend along a portion or an entirety of a length or width of the cover 350. As shown in FIG. 3A, the support bands 360 can extend in a generally longitudinal direction. As shown in FIG. 3B, the support bands 360 can extend in a generally circumferential direction. Each of the support bands 360 can include one or more bends 362 to facilitate bending along the length of the support band 360. The support bands 360 can be advanced and retracted with the cover 350, independent of any other wires (e.g., wires 130, 140, not shown in FIGS. 3A and 3B).

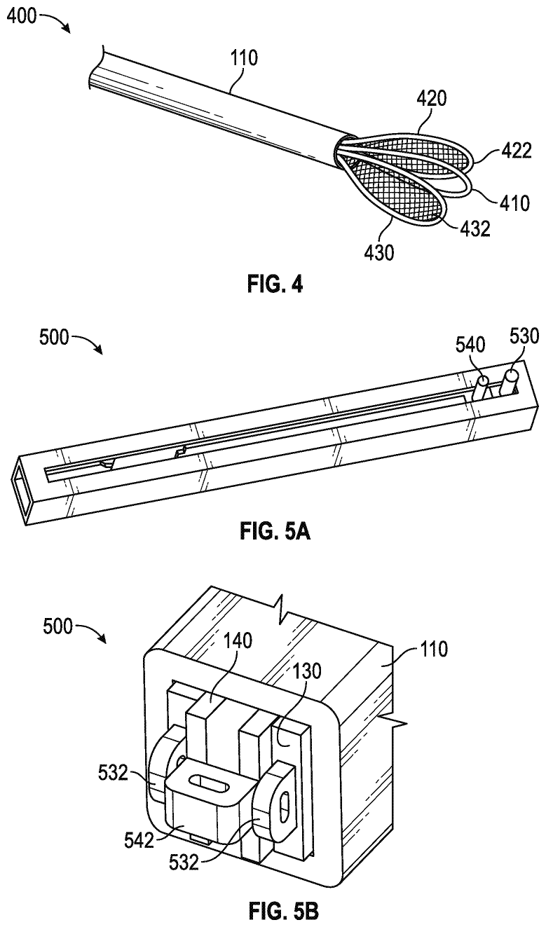

According to some embodiments, for example as shown in FIG. 4, an extraction device 400 can include a delivery shaft 110 having a lumen and a distal end and a dissection tool 410 distal to the distal end of the delivery shaft 110. The dissection tool 410 can be a wire loop or any other mechanism for dissecting a lens. The dissection tool 410 can be aligned along a central axis of the delivery shaft 110. A first capture portion 420 can be positioned on a first side of an axis of the delivery shaft 110. The first capture portion 420 can include a first cover 422. A second capture portion 430 can be positioned on a second side of an axis of the delivery shaft 110. The second capture portion 430 can include a second cover 432. The first capture portion 420 and the second capture portion 430 are configured to move toward the axis upon actuation and define an enclosed space between the first cover 422 and the second cover 432. The first capture portion 420 and the second capture portion 430 can be actuated, for example, by at least partial retraction into the delivery shaft 110. The dissection tool 410 can be used to dissect the lens after actuation of the first capture portion 420 and the second capture portion 430. Alternatively, or in combination, the dissection tool 410 can be retracted into the delivery shaft 110 prior to actuation of the first capture portion 420 and the second capture portion 430. The first cover 422 and the second cover 432 can be an elastomer.

According to some embodiments, for example as shown in FIGS. 5A and 5B, deployment and retraction of components described herein can be achieved by an interface tool 500. The interface tool 500 can include a first control mechanism 530 and the second control mechanism 540. The first control mechanism 530 can connect, via a first interface 532, to the first wire 130 or another component described herein. The second control mechanism 540 can connect, via the second interface 542, to the second wire 140 or another component described herein. Actuation of the first control mechanism 530 and the second control mechanism 540 can separately controllably deploy, retract, and/or rotate components attached thereto. According to some embodiments, the cross-section of the slide mechanism can be various shapes including circular (e.g., one tube within another which allows for rotational motion in addition to translational motion).

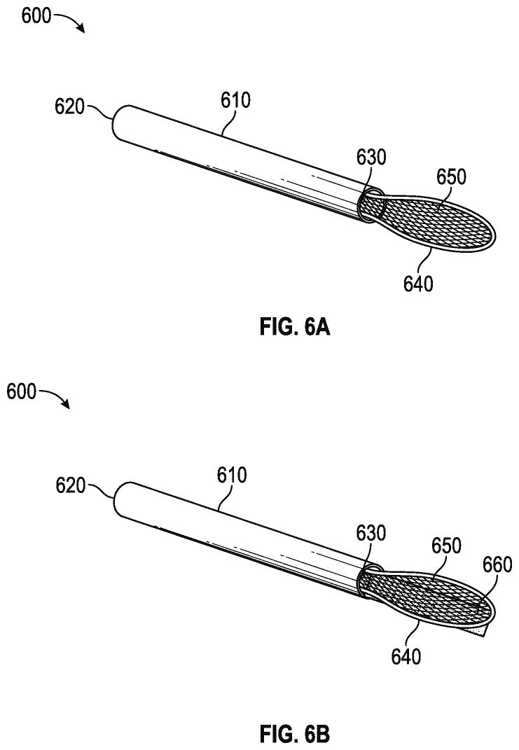

According to some embodiments, for example as shown in FIGS. 6A and 6B, an extraction device 600 can include a delivery shaft 610 having a lumen and a distal end. The extraction device 600 can further include an infusion port 620 for connecting a syringe or other fluid source to an irrigation port 630 at a distal end of the delivery shaft 610. The extraction device 600 can further include a loop 640 attached to the distal end of the delivery shaft 610. The loop 640 can be configured to be positioned under a lens to pull the lens out of the eye as irrigation and/or aspiration is provided from the irrigation port 630. A cover 650 can extend across an opening of the loop 640 and attach at a periphery of the loop 640. The extraction device 600 can further include a blade 660 that extends from an interior portion of the delivery shaft 610 to extend across at least a portion of an opening of the loop 640. The blade 660 can perform a cutting action upon the lens or other tissue encountered along its path. The blade 660 can be metal, silicone, or a rigid material. The blade 660 can be advanced until it reaches a distal end of the loop 640. The blade 660 can travel along a track defined by the loop 640. An interior space can be defined between the blade 660 and the cover 650 for capturing materials. The loop 640 and the blade 660 can be retracted into the lumen of the delivery shaft 610, along with any materials captured therein.

According to some embodiments, a method for using extraction devices described herein includes making an incision (e.g., an incision of less than 4 millimeters (mm)) to enter an anterior chamber of an eye. A capsulorhexis is performed. The lens is hydrodissected and/or hydrodelineated so that the lens changes position from being entirely within the natural capsular bag to being partially or completely displaced from the capsular bag. Viscoelastic may be used to further position the lens for optimum approach by an extraction device. The extraction device is introduced through the incision and advanced forward. An encircling structure (e.g., capture region 120) is introduced through the lumen of the device and advanced towards the lens. The encircling structure is positioned so that it surrounds the lens. Further deployment of the encircling structure may allow for multiple elements to expand and further encompass the lens. A second structure (e.g., cover 150) is advanced from the lumen of the device distally. The second structure covers the encircling structure from proximal to distal and substantially conforms to the encircling structure geometry. The encircling structure is retracted back into the lumen and may or may not twist as it is being retracted so that the lens is segmented into pieces smaller than the original whole lens. The encircling structure may or may not be advanced and retracted several times to further segment the lens. The second structure is then retracted in whole or in segments so that any remaining lens material within the structure is retracted into the lumen. The second structure can expand distally to allow for all the lens material to fit into the lumen. The entire device is removed from the anterior chamber of the eye. A second suction device is used to remove any remaining lens materials. The lens bag is reformed with viscoelastic to receive an artificial intraocular lens for reversal of aphakia and refractive correction.

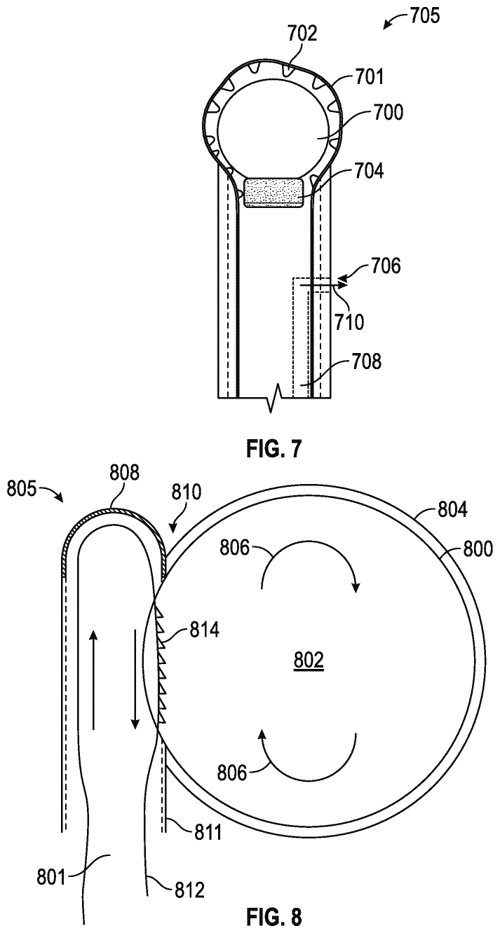

According to some embodiments, for example as shown in FIG. 7, nitinol wire(s) 701 of an extraction device 705 can grab the lens 700. The wires 701 can be surrounded by a mesh or other means to encapsulate the lens. The wires can contain teeth 702 or other means to firmly hold onto the lens 700. The wires 701 can spin the lens 700 in the plane that is shown if the distal part of the wire is pulled while the proximal part is advanced. This can be done using a wheel or other mechanism, or the wire can simply tension the lens against the lumen when it is retracted slightly. A recessed area contains a rotating element 704 that is abrasive or sharp to break the lens 700 into pieces. As the lens 700 is pulled into the recessed area by the wire 701, the abrasive rotating element 704 is actuated mechanically with gears or another mechanism to break up the lens. The lumen of the device has another port 706 and tube 708 within it that allows for fluid irrigation when connected to a pressurized fluid source. Fluid 710 can be aspirated by connecting the main lumen to a vacuum mechanism.

According to some embodiments, for example as shown in FIG. 8, one or more nitinol wires 800 of device 805 grabs the lens 802. The wires 800 can be surrounded by a mesh 804 or other means to encapsulate the lens. The wires can have features like the ones described for FIG. 1A. The wires can spin the lens as shown by the large arrows 806 or tension the lens against a recessed space within the lumen 801. Note that the end 808 of the lumen 801 is capped and the opening 810 is in the wall 811 of the tube. Within the lumen there is wire 812 with jagged cutting edges 814 (e.g., like a bandsaw). When the lens is tensioned against the bandsaw, the bandsaw can be mechanically actuated to break up the lens. This design can also have an irrigation and aspiration port.

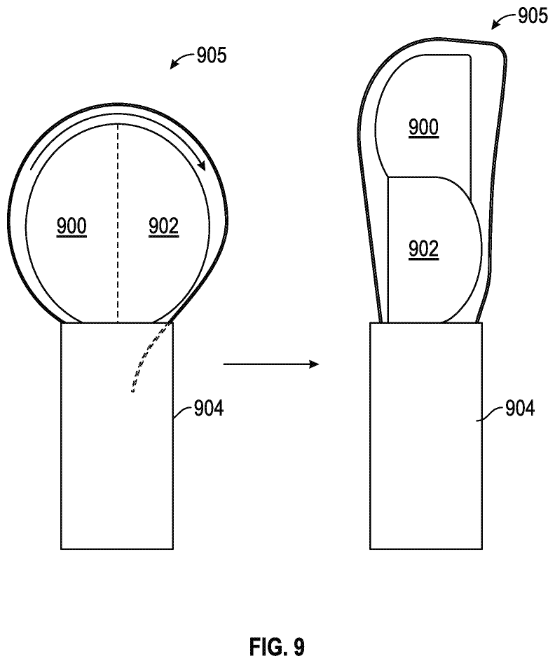

According to some embodiments, for example as shown in FIG. 9, first and second structures 900 and 902 of a device 905 can expand distally to allow for all the lens material to fit into the lumen. In this way, lens material can be squeezed and extended to be able to fit into the lumen 904 of the device.

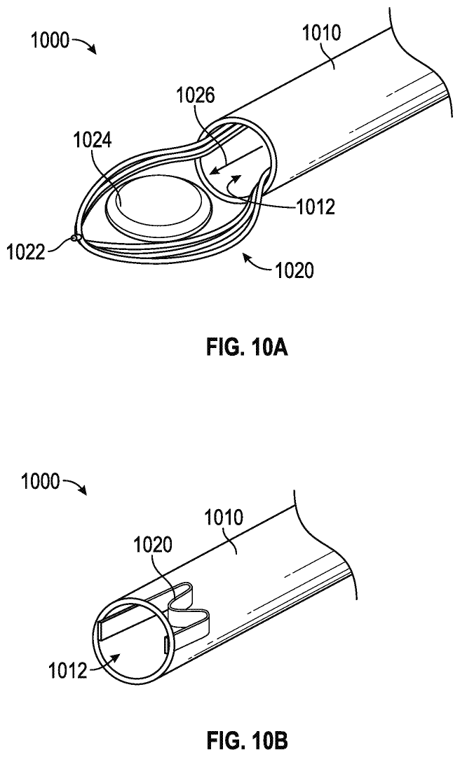

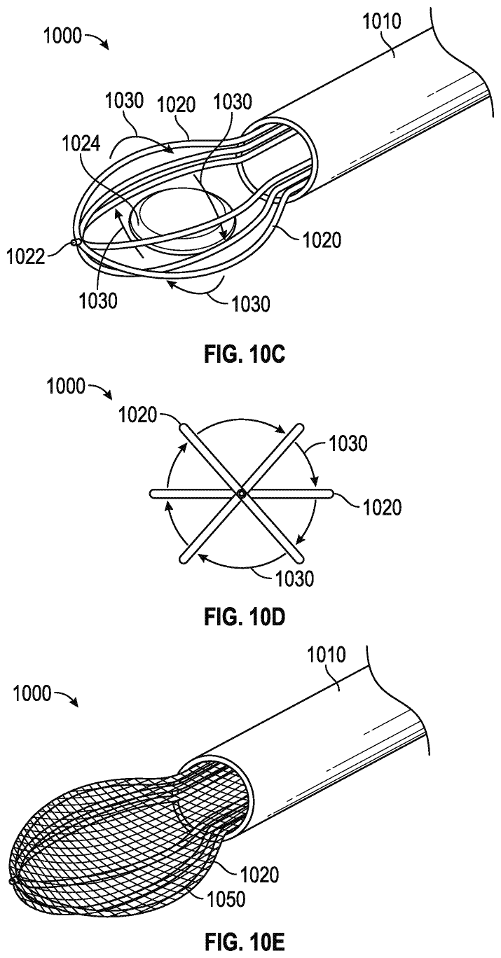

According to some embodiments, for example as shown in FIGS. 10A-10E, an extraction device 1000 can include a plurality of rings 1020 that extend in a direction 1026 from a lumen 1012 of a delivery shaft 1010 to encapsulate a sample or specimen 1024 (e.g., some or all of a lens). Rings 1020 may, for example, be concentric wire rings that are pivotally bound at a joint 1022 at a distalmost end. In the configuration of FIG. 10A, rings 1020 have been deployed out of the lumen 1012 (e.g., by a manually or automatically controlled mechanism such as a push rod as described above in connection with FIGS. 5A and 5B).

As shown in FIG. 10B, prior to deployment, rings 1020 may be folded within the distal end of lumen 1012. In this way, rings 1020 can be stored within shaft 1010 during insertion through an incision. As shown in FIG. 10C, rings 1020 may be rotated (e.g., in the direction of arrows 1030) relative to each other such that rings 1020 encapsulate and substantially surround sample 1024. FIG. 10D shows a top view of rings 1020 following rotation of the rings in directions as indicated by arrows 1030 to an encapsulated configuration.

Following encapsulation of the sample, a mesh cover or bag 1050 as discussed herein may be extended over rings 1020 (e.g., using guides similar to guides 160 above) to substantially surround and encapsulate the sample 1024 within bag 1050. In some embodiments, once sample 1024 is encapsulated within rings 1020 and bag 1050, rings 1020 may be pulled back into lumen 1012. As rings 1020 are withdrawn into lumen 1012, rings 1020 may cut sample 1024 into fragments to aid in withdrawing the sample into lumen 1012. After cutting of sample 1024 with rings 1020, bag (or cover) 1050 may be withdrawn into lumen 1012 to retrieve and extract the sample.

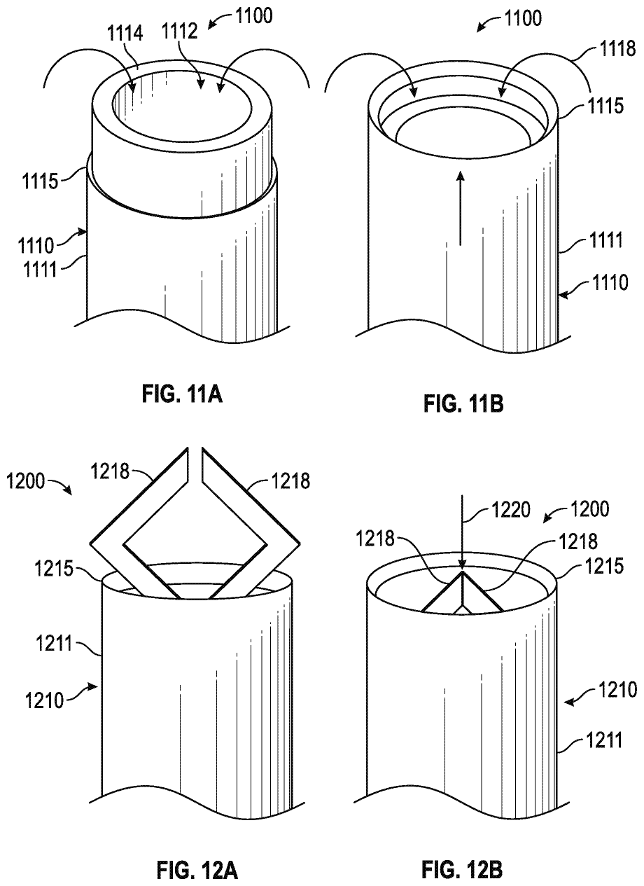

According to some embodiments, for example as shown in FIGS. 11A and 11B, an extraction device 1100 can include a shaft 1110 having an inner cylindrical shaft structure 1114 with a lumen 1112 and an outer shaft structure 1111 with a sharp cutting edge 1115 at a distalmost end thereof. As shown in FIG. 11B, cutting edge 1115 of outer shaft structure 1111 can be extended beyond the distalmost end of inner shaft structure 1114 to excise tissue. Suction can be provided that draws tissue excised by cutting edge 1115 into lumen 1112 as indicated by arrows 1118.

According to some embodiments, for example as shown in FIGS. 12A and 12B, an extraction device 1200 can include a shaft 1210 including an outer shaft structure 1211 with a sharp cutting edge 1115 at a distalmost end thereof. As shown in FIG. 12A, gripping structures such as forceps 1218 may be extended from within shaft 1210 to grip tissue to be excised. As shown in FIG. 12B, as forceps 1218 are withdrawn into shaft 1210 in direction 1220, the tissue held by forceps 1218 can be cut free by cutting edge 1215. The forceps 1218 and/or suction can be provided that draws tissue excised by cutting edge 1215 into shaft 1210 for removal of the tissue.

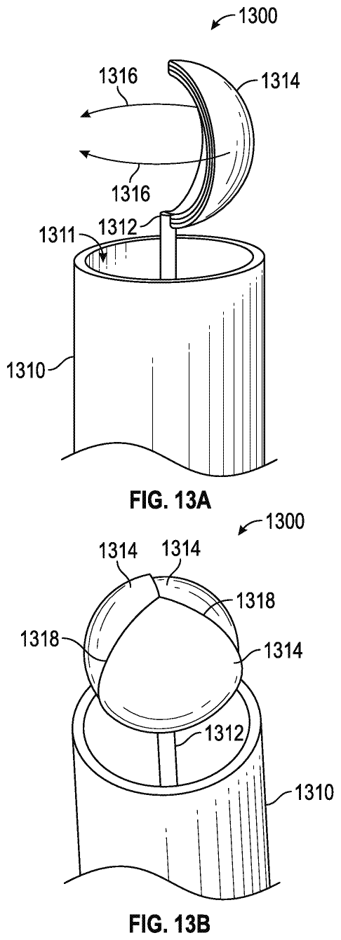

According to some embodiments, for example as shown in FIGS. 13A and 13B, an extraction device 1300 can include a shaft 1310 having a lumen 1311 and an excision member that extends therefrom. The excision member may include a control shaft 1312 and one or more layered cutting and encapsulation leaves 1314. In the layered configuration of FIG. 13A, the excision member may have a concave scoop shape with a reduced profile for insertion through an incision. Layered cutting and encapsulation leaves 1314 may be reticulated relative to each other about an axis as indicated by arrows 1316 to create an enclosure for a lens or portion thereof as shown in the encapsulation configuration of FIG. 13B.

In some embodiments, one or more of leaves 1314 may have a cutting edge 1318 thereon that, when that leaf 1314 is rotated about the axis of, for example, shaft 1312, cuts through and excises the tissue to be encapsulated. In other embodiments, edges of leaves 1314 may be blunt edges that slide between the lens and its surrounding tissue to encapsulate substantially the entire lens prior to removal. In some embodiments, a cutting and/or suction mechanism can be applied within the encapsulated region formed by leaves 1314 (e.g., via an inner lumen of shaft 1312 into the encapsulated region to break up the encapsulated lens and suction out the broken up lens before re-stacking or re-layering leaves 1314 for withdrawal of device 1300.

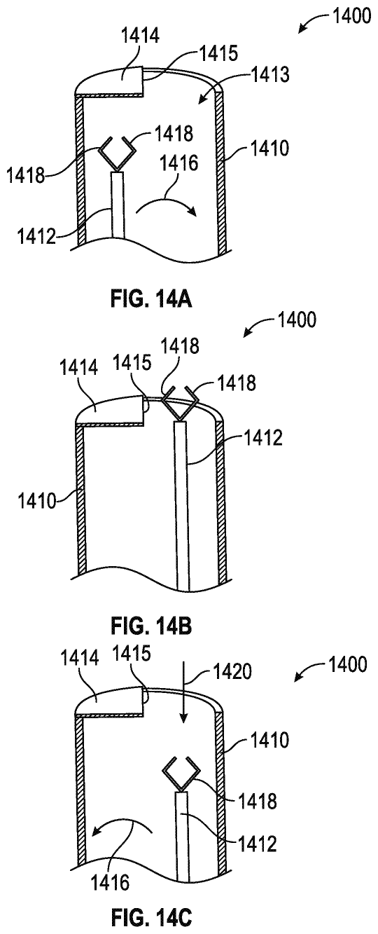

According to some embodiments, for example as shown in FIGS. 14A-14C, an extraction device 1400 can include a shaft 1410 having a lumen 1413 with a partial lid structure 1414 for the lumen. Partial lid structure 1414 may include a sharp cutting edge 1415. In the example of FIG. 14A, partial lid 1414 extends perpendicularly to the axis of shaft 1410 to cover a portion of lumen 1413. However, this is merely illustrative, and partial lid 1414 (and cutting edge 1415) can be positioned at angles other than 90 degrees with respect to the axis of shaft 1410.

As shown in FIG. 14A, forceps 1418 may be movably disposed within lumen 1413 for grasping or gripping of tissue. In the embodiment of FIGS. 14A-14C, a twisting action may be performed to excise tissue using an axis that is off-center to the cutting edge 1415. For example, as shown in FIG. 14B, forceps 1418 may be extended out of lumen 1413 by inner shaft 1412 to grasp tissue to be excised. The tissue may be grabbed, suctioned, and/or drawn into lumen 1413 (as indicated by arrow 1420 of FIG. 4C) and moved relative to the cutting edge 1415 to cause an excision. The tissue can be moved by fixing the cutting edge and rotating forceps 1418 (e.g., as indicated by arrow 1416 of FIGS. 4A and 4C) and/or by rotating shaft 1410 to rotate cutting edge 1415 relative to the tissue that is held by forceps 1418 (e.g., to reduce the potential of lens movement or rotation during excision that can damage the capsule). Grasping, suctioning, and/or rotating of tissue relative to cutting edge 1415 can be repeated to excise portions of a lens or other tissue until all of the desired tissue is removed.

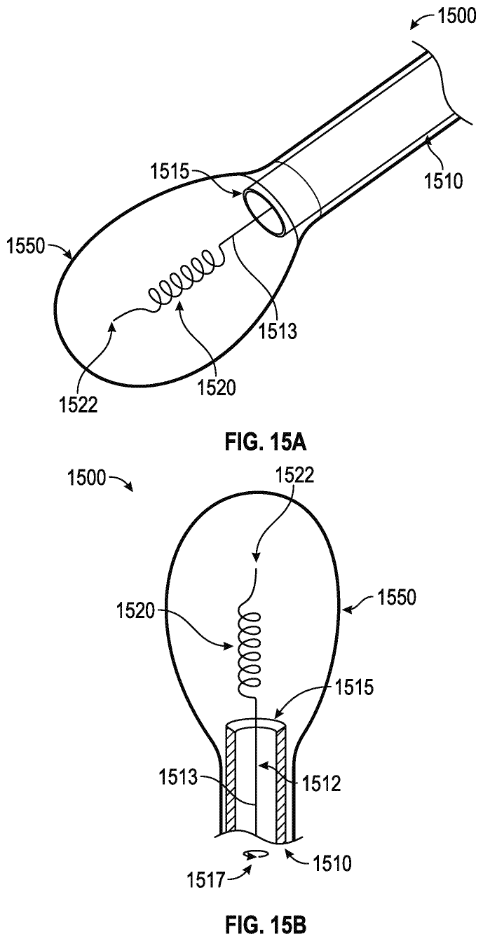

According to some embodiments, for example as shown in FIGS. 15A and 15B, an extraction device 1500 can include a shaft 1510 having a lumen 1512 and a wire 1513 having a helical wire section 1520. Wire 1513 may be extendible from within the lumen 1512 at the distalmost end thereof. Shaft 1510 may include a sharp cutting edge 1515 at the distalmost end thereof. Wire 1513 may have a sharp piercing tip 1522 so that helical wire section 1520 can be rotated into a sample in a manner similar to a corkscrew to penetrate and grip the sample to be removed.

As shown, a mesh cover or bag 1550 may be deployed from shaft 1510 (in a manner similar to the cover extension operations as discussed above in accordance with one or more embodiments) to encapsulate the sample to be removed. FIG. 15B shows a cross-sectional view of shaft 1510 showing how cutting edge 1515 may include an angled surface at the end of shaft 1510 relative to the axis of shaft 1510.

In the example of FIGS. 15A and 15B, shaft 1510, having cutting edge 1515, may be inserted into the anterior chamber of a patient's eye to deploy wire 1513 (e.g., a metallic wire as described herein) having piercing tip 1522 and a helical section 1520. As the wire 1513 is deployed, the wire tip 1522 pierces a specimen to be removed and may be rotated as indicated by arrow 1517 to envelop into the specimen. Bag 1550 (e.g., a silicone bag or other cover) may be deployed around the specimen and pulled back into shaft 1510. This extraction (e.g., the pull-back of bag 1550) may cause edge 1515 to cut through the specimen to allow the specimen to enter into lumen 1512.

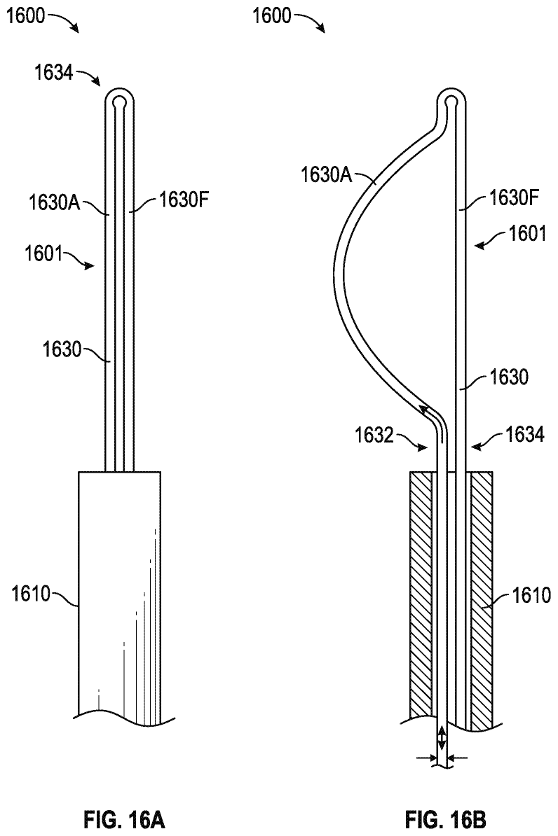

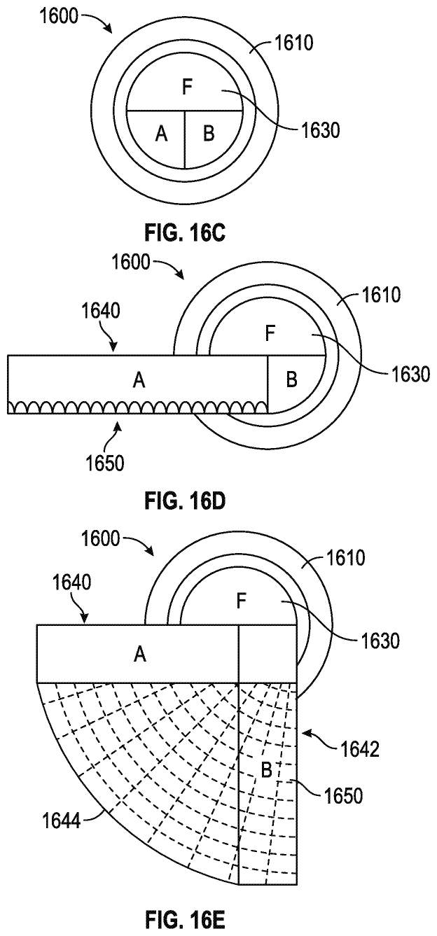

According to some embodiments, for example as shown in FIGS. 16A-16E, an extraction device 1600 may include a shaft 1610 having an inner lumen from which a wire 1630 extends. Wire 1630 may be a deployable wire that is extendible from the distalmost end of shaft 1610 or may be permanently deployed. Wire 1630 may have a sliding side 1632 and a fixed side 1634.

FIGS. 16A and 16B show device 1600 with a tip 1601 in a fully closed state (FIG. 16A), and an open state (FIG. 16B). Device 1600 may be a device for cataract extraction which is configured to enter the eye of a patient through a small incision in a somewhat cylindrical shape as in the configuration of FIG. 16A. As shown, device 1600 may have a tubular shaft 1610 which houses the cataract capturing components (labeled as segments 1630A, 1630B, and 1630F). Segments 1630A, 1630B, and 1630F may be fully retractable into tubular shaft 1610 (e.g., to make entering the eye easier), or may remain outside tubular shaft 1610 as depicted in FIG. 16A. Segments 1630A, 1630B, and 1630F may be joined at a hinge point 1639.

Once in the eye, tip 1601 of device 1600 may be forced to change shape by sliding segment 1630A to make it deform into a bow shape. This deformation may open the mouth of a basket formed to scoop up the lens. Segment 1630F may be fixed from moving in relation to segment 1630A. In addition to opening the mouth of the basket by deforming segment 1630A, the length of the basket may extended by sliding segment 1630B (see, e.g., FIGS. 16C-16E) in the same manner as segment 1630A, but in a different direction (e.g., 90 degrees different) from that of segment 1630A, causing segment 1630B to deform into a long bow shape.

As segment 1600B is deformed, segment 1600B may stretch open a bag or net-like cover structure 1650 as shown in FIGS. 16D and 16E. With segments 1630A and 1630B fully extended as in the configuration of FIG. 16E, device 1600 may look somewhat like a butterfly net, which can be used to capture the lens to be removed. After capturing the lens, the entire assembly may be pulled down into tubular shaft 1610 to crush the lens enough to reduce the size, such that it can be withdrawn through the small incision in the eye.

The top view of FIG. 16C shows device 1600 in a fully closed state, as seen in FIG. 16A from the side view. In the top view of FIG. 16D, segment 1630A has been forced to deform such that the mouth 1640 of the basket opens. The configuration of FIG. 16D corresponds to that of FIG. 16B which shows, from the side, the bowed shape of segment 1630A, and how segment 1630A was slid up the tubular shaft while segment 1600F was held fixed in position. In the configuration of FIG. 16E, segments 1630A and B have been forced to deform such that the length 1642 of the basket 1644 is formed and the bag or net structure 1650 was deployed.

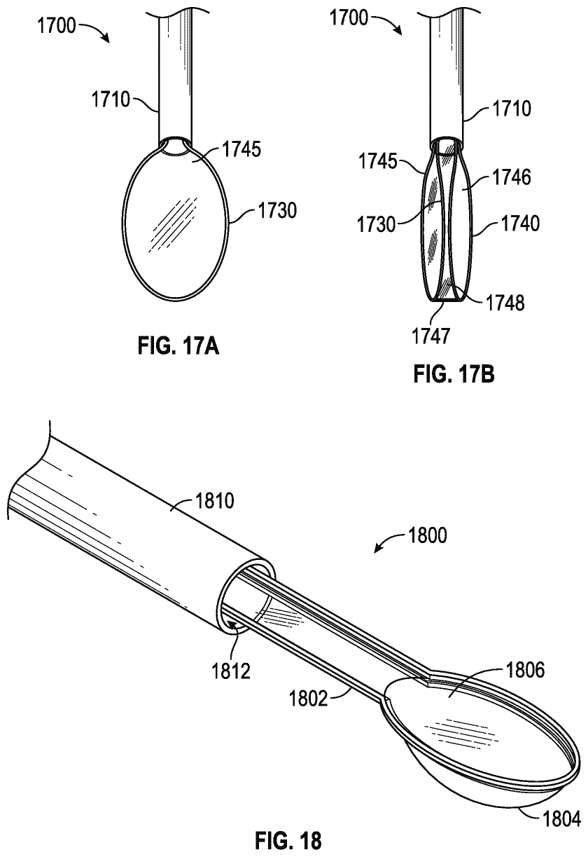

According to some embodiments, for example as shown in FIGS. 17A and 17B, an extraction device 1700 may include a first ring 1730 and a second ring 1740 that are extendible from a shaft 1710. Ring 1730 (e.g., a metal ring such as a NiTi ring) may be provided with a film such as a PTFE film 1745 that spans the ring. Second ring 1740 (e.g., a metal ring such as a NiTi ring) may be an open ring having an opening 1746 that is free of any film.

In the embodiment of FIGS. 17A and 17B, shaft 1710 may form an inserter that can be inserted into an incision (e.g., a 2.75 mm incision). For example, in some embodiments, the internal diameter of shaft 1710 may be 2.2 mm and the outer diameter of shaft 1710 may be 2.4 mm. Rings 1730 and 1740 may be deployed from the inserter behind a hydrodissected lens. Rings 1730 and 1740 may be opened up and separated from each other slightly (e.g., to a distance of 2 mm apart). Rings 1730 and 1740 may be bridged by a film 1747 (e.g., PTFE film) with ring 1730 covered with, for example, PTFE 1745 and ring 1740 open (i.e., not covered by PTFE or other films). The lens may then be guided through the open ring 1740 to sit against the film 1745 that bridges the other ring 1730 (e.g., so that the natural lens is sitting in an open basket 1748 formed by rings 1730 and 1740 and films 1745 and 1747). Once the cataract/lens is in place in the basket 1748, the two rings can then be separated apart from each other by another 2 mm so that the entire lens is in the “basket” 1748. Once the cataract is in the basket 1748, a “lid” (not shown) to the basket may be slid over the open space 1746 in ring 1740. The lid (e.g., formed by an additional ring with, e.g., PTFE covering) can slide in on small slots within ring 1740 for a smooth and controlled deployment of the lid. In some embodiments, one or more additional rings (e.g., 50 micron rings) may be deployable into and/or out of the basket 1748 for further cutting of a sample/lens therein.

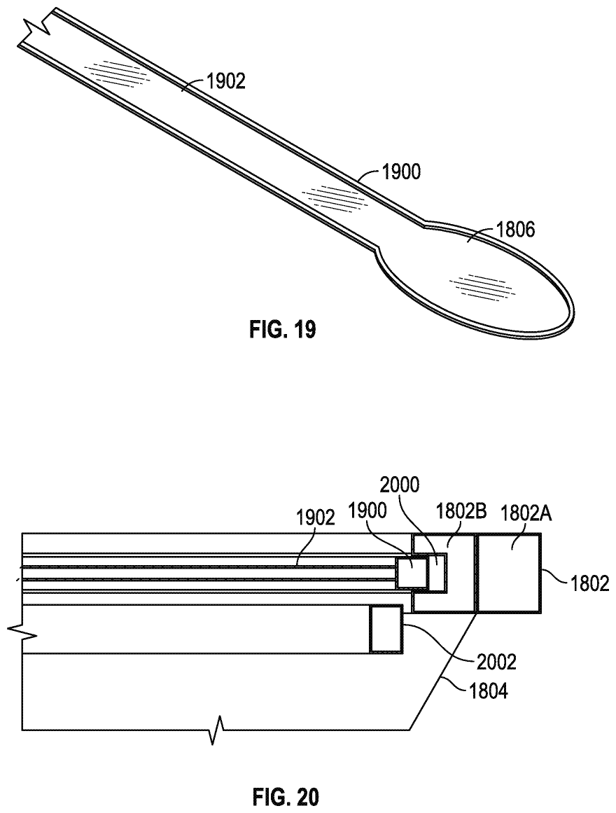

In the example of FIGS. 17A and 17B, film 1745 is shows as a substantially planar film. However, in some embodiments, a multi-ring scoop extraction device may be provided with a ring having a film that forms a basket shape. An example embodiment of a multi-ring scoop extraction device 1800 is shown in FIGS. 18-23.

According to some embodiments, for example as shown in FIGS. 18-23, an extraction device 1800 may include ring frames that are extendible from a lumen 1812 of a shaft 1810. In the example of FIGS. 18-23, extraction device 1800 includes a first (e.g., nitinol) frame 1802 that has a polymer or mesh bag 1804 attached thereto.

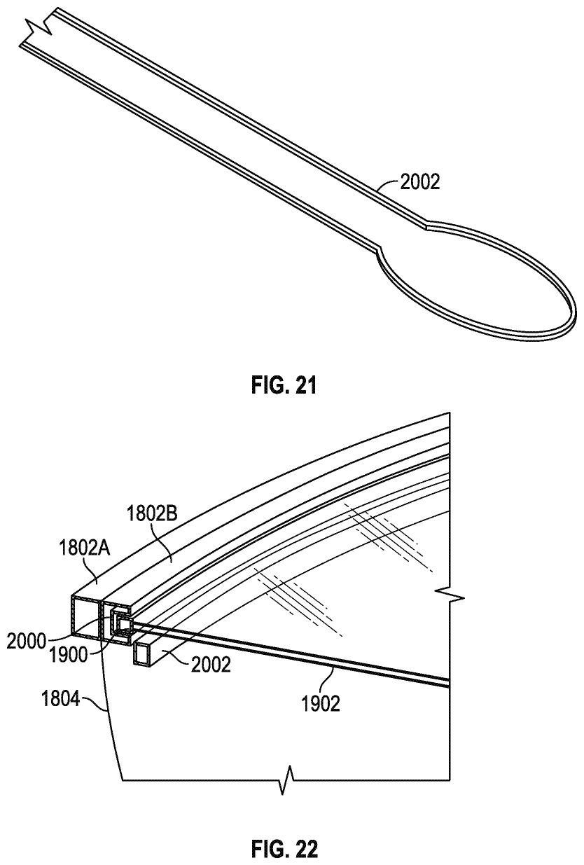

Device 1800 may also include an additional (e.g., nitinol) frame 2002 (see, e.g., FIGS. 20 and 21) that can be deployed into and out of lumen 1812 independently of the first frame 1802. Initially, frame 2002 may be deployed when the initial bag/frame 1804/1802 component is deployed so that second frame 2002 sits just below frame 1802 as shown in the cross-sectional views of FIGS. 20 and 22.

Device 1800 may also include a third frame 1900 (e.g., a nitinol frame) with a covering 1902 that forms a lid 1806 for basket 1804 when third frame 1900 is deployed. Covering 1902 may be a polymer or mesh covering or, in some embodiments, may contain relatively more rigid or solid material to provide additional strength and structure to frame 1900 for cutting of lens tissue as discussed in further detail hereinafter. In use, one or more of the following tasks may be performed.

In a first task, after a capsulorhexis has been performed and a lens of a patient's eye has been hydrodissected out of the natural position (e.g., so that the lens is sitting on the equator and partially in the iris plane), an introducer such as shaft 1810 may be inserted into the patient's eye (e.g., via an incision). In a second task, frame 1802 with bag 1804 and frame 2002 may be deployed into the anterior chamber of the eye. In a third task, the lens may be approached and guided partially into the bag 1804 so that, for example, half the lens is in this artificial bag 1804. In a fourth task, lid 1806 on frame 1900 may then be introduced such that lid 1806 and frame 1900 transect (e.g., bisect) the lens so that a portion (e.g., substantially half) of the lens is in encapsulated within a cavity defined by the bag 1804 and lid 1806 and a remaining portion (e.g., a remaining half) is outside of the cavity. In a fifth task, frame 2002 that was introduced into the eye along with frame 1802 may be withdrawn or retracted from within the cavity into the lumen 1812 of the introducer 1810 to further segment the portion of the lens that is sitting in the cavity between artificial bag 1804 and lid 1806. In a sixth task, the entire complex of all pieces in bag 1804 (e.g., in the cavity defined by bag 1804 and lid 1806) may then be retracted into the introducer 1810 to remove the portion of lens that is encapsulated therein. Introducer 1810 may then be removed from the eye and the segmented pieces are removed from the bag. The first through sixth tasks may be repeated for, for example, the other half lens that remains in the eye. Although the example tasks above have been described with a portion of the lens being encapsulated in the cavity defined by bag 1804 and lid 1806, in some scenarios, the entirety of a lens may be encapsulated within the cavity, transected by one or more frames 2002, and removed by withdrawing frames 1802 and 1900 into introducer 1810 (e.g., without transecting the lens with lid frame 1900 while extending the lid frame 1900).

As shown in the cross-sectional view of FIG. 20, lid frame 1900 may ride on an inside railing formed by a recess 2000 of the first frame 1802 (e.g., a recess in an inner portion 1802A of frame 1802 disposed interior to an outer frame portion 1802B) that contains the bag 1804. The leading end of the lid frame 1900 may be sharp so that frame 1900 can transect (e.g., bisect) the lens easily. In some embodiments, lid frame 1900 may be formed from two or more frame segments to facilitate advancement of the lid. For example, a segmented frame 1900 may be provided to allow for the lid frame 1900 to ride along the structure of the first frame 1802 with more precision (e.g., a bisected frame 1900 may ride on the rails of the first frame 1802 more easily than a monolithic frame).

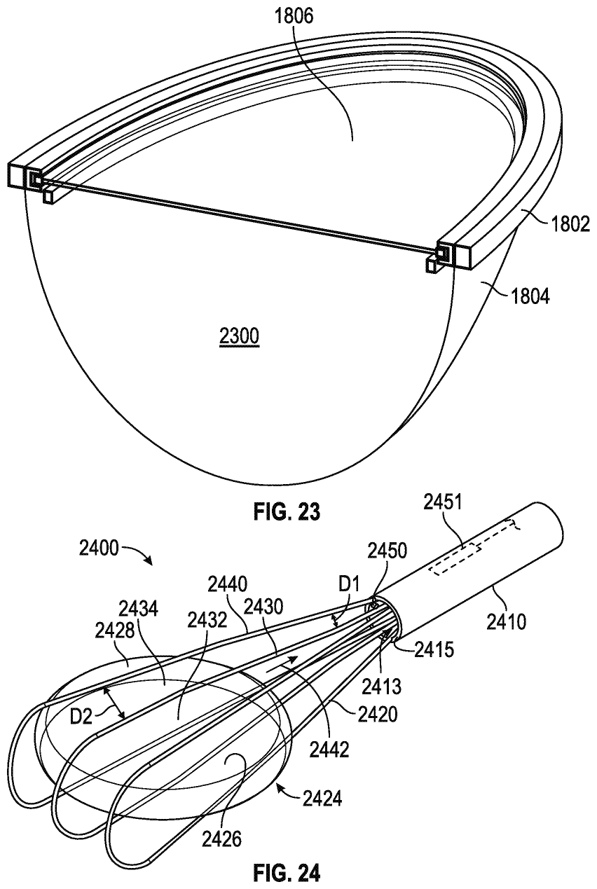

In some embodiments, frame 2002 that is retracted after bagging the lens might be one of many such intermediate frames that are deployable and retractable such that the half lens that is bagged can be segmented multiple times with the multiple intermediate frames. For example, a plurality of frames 2002 may be stacked on top or next to each other for independent deployment and retraction for cutting actions. Although nitinol is often mentioned herein as an example material for frames and/or other components, frames 1802, 1900, and/or 2000 may be formed from nitinol or other metals or polymers in various embodiments. FIG. 23 shows a full cross-sectional perspective view of the cavity 2300 that is defined by and enclosed between bag 1804 and lid 1806 when deployed. Cavity 2300 may hold substantially half of a lens of a patient's eye as described herein for further dissection and removal.

Bag 1804 and/or lid 1806 can contain elastic or inelastic polymer or mesh materials. Shaft 1810 (sometimes referred to as an introducer or an injector) may be composed of metal, polymer or glass materials. The distal end of the introducer, in one or more embodiments, may contain flexible materials that expand when the bag is retracted back into the introducer. Shafts such as shaft 110 etc. described herein may be formed from sections of hypodermic tubing, sometimes referred to as a hypotube, according to some embodiments.

Although bag 1804 and lid 1806 are shown in various examples as continuous sheets of material attached to their respective frames, this is merely illustrative. In some implementations, bag 1804 and/or lid 1806 may be provided with one or more openings (not explicitly shown). The openings may be cutouts from a continuous sheet implemented bag or lid or may be openings in mesh sheets attached to the respective frames that form bag 1804 and/or lid 1806.

In configurations in which bag 1804 and/or lid 1806 are provided with one or more openings, after some or all of a lens of a patient's eye has been secured between bag 1804 and lid 1806 within the anterior chamber of the patient's eye, frames 1802 and/or 1900 may be withdrawn into delivery shaft 1810 such that parts of the secured lens (or portion thereof) are forced to exit the bag and/or lid through the openings into the anterior chamber as the bag and/or lid is being withdrawn into the distal end of the tube. In this way, bag 1804 and/or lid 1806 may be configured to act as a strainer for the lens (or portion thereof) that, upon withdrawal of frames 1802 and/or 1900 into the distal end of delivery shaft 1810, cause some or all of the lens come out through the openings as a ground or strained material that can be later removed (e.g., via suction) from the anterior chamber.

In configurations in which bag 1804 and/or lid 1806 are provided with one or more openings, a method of removing some or all of the lens of a patient's eye may include extending a first frame (e.g., frame 1802) having an attached flexible bag structure (e.g., bag 1804) with a plurality of openings from a distal end of a delivery shaft (e.g., shaft 1810) into an anterior chamber of an eye of a patient such that the flexible bag structure at least partially surrounds at least a portion of a lens of the eye of the patient; extending a second frame (e.g., frame 1900) having an attached lid structure (e.g., lid 1806) along the first frame to secure the at least the portion of the lens between the flexible bag structure and the lid structure; and withdrawing the first and second frames into the distal end of the delivery shaft to strain the at least the portion of the lens through the plurality of openings in the flexible bag structure into the anterior chamber.

The method may also include extending a third frame (e.g., frame 2002) into the eye of the patient together with the first frame; and, prior to withdrawing the first and second frames, withdrawing the third frame to transect the secured at least the portion of the lens, in one or more embodiments.

The method may also include suctioning the strained at least the portion of the lens from the anterior chamber, in one or more embodiments.

According to some embodiments, for example as shown in FIG. 24, an extraction device 2400 can include a delivery shaft 2410 having a lumen 2413 and a distal end 2415 from which one or more wires can extend. For example, as shown in FIG. 24, extraction device 2400 may be implemented as a three-wire snare having first wire 2420, second wire 2430, and third wire 2440. First wire 2420, second wire 2430, and third wire 2440 may each be a wire loop formed from metal (e.g., nitinol), plastic, and/or a shape memory material.

First wire 2420, second wire 2430, and third wire 2440 may be extended from distal end 2415 of delivery shaft 2410 and positioned to lasso or surround lens 2424 (e.g., within the eye following hydrodissection or hydrodelineation of the lens). Once first wire 2420, second wire 2430, and third wire 2440 are positioned around the lens as shown in FIG. 24, first wire 2420, second wire 2430, and third wire 2440 can be retracted back into lumen 2413 of delivery shaft 2410 (e.g., in direction 2442). As first wire 2420, second wire 2430, and third wire 2440 are retracted into lumen 2413, first wire 2420, second wire 2430, and third wire 2440 may be pulled through lens 2424 to split lens 2424 into four pieces 2426, 2428, 2430, and 2432. The four pieces 2426, 2428, 2432, and 2434 of lens 2424 may then each be removed from the eye (e.g., with a forceps that grabs and/or crushes the lens piece).

As shown in FIG. 24, when first wire 2420, second wire 2430, and third wire 2440 are in an extended position with respect to distal end 2415 of delivery shaft 2410, first wire 2420, second wire 2430, and third wire 2440 may be positioned such that the distance between each wire increases with increasing distance from distal end 2415 (e.g., from a first distance D1 near distal end 2215 of shaft 2410 to a second distance D2 that is greater than D1 near a distalmost end of the wire loops). First wire 2420, second wire 2430, and third wire 2440 may be positioned together within lumen 2413 and then separated during or after extension from lumen 2413.

For example, in one implementation, delivery shaft 2410 may include one or more features such as feature 2450 (e.g., an integral or attached extension from the inner surface of delivery shaft 2410 in lumen 2413) that causes first wire 2420, second wire 2430, and third wire 2440 to separate from each other as they are extended from distal end 2415 (e.g., as adjacent wires slide along opposing sides of feature 2450). Feature 2450 may be a protrusion extending from an inner surface of the delivery shaft in the lumen that causes the first, second, and third wire loops to separate upon extension from within the lumen.

As another example, after extension of first wire 2420, second wire 2430, and third wire 2440 together (e.g., in contact with each other in a substantially flattened configuration) from lumen 2413, one or more slidable separators 2451 may be actuated within or extended from lumen 2413 between adjacent wires to cause the adjacent wires to separate into the position shown in FIG. 24. Slidable separator 2451 may be moved between at least two of the first, second, and third wire loops to separate the at least two of the first, second, and third wire loops.

As another example, first wire 2420, second wire 2430, and third wire 2440 may be heat set wires such as nitinol heat set wires that are conditioned to separate after being extended a predetermined distance from within lumen 2413. For example, first, second, and third wire loops 2420, 2430, and 2430 may be heat set wire loops configured to self separate upon extension to a predetermined distance from the distal end of the delivery shaft.

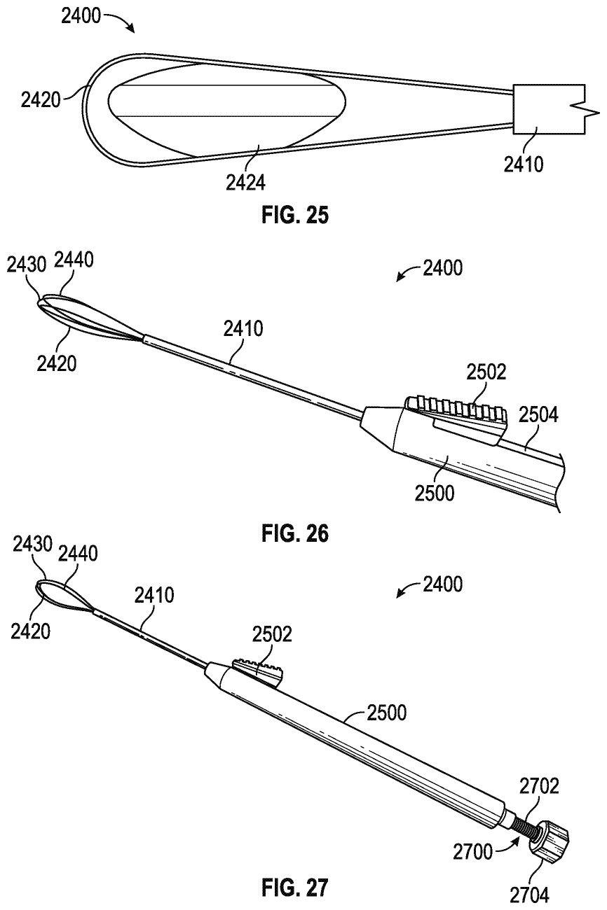

In various implementations, first wire 2420, second wire 2430, and third wire 2440 may be extended from lumen 2413 at the same time or at different times (e.g., using a common extension mechanism or separate extension mechanisms to push the wires out of the lumen). FIG. 25 shows a side view of device 2400 with the wires extending around lens 2424. It should be appreciated that, although wires 2420, 2430, and 2440 have been referred to as a first wire, a second wire, and a third wire, this is merely for convenience and any of wires 2420, 2430, and 2440 can be referred to as a first wire, a second wire, or a third wire (or wire loop).

FIG. 26 shows a broader view of extraction device 2400 in which delivery shaft 2410 can be seen extending from a handle 2500 of device 2400. As shown, delivery shaft 2410 may be a substantially cylindrical tube attached at a proximal end to handle 2500. Handle 2500 may include a slider tab 2502, moveable by a user within a groove 2504, for deployment and retraction of wires 2420, 2430, and 2440 from delivery shaft 2410. In the configuration shown in FIG. 26, slider tab 2502 is in a forwardmost position and wires 2420, 2430, and 2440 are fully extended from shaft 2410. Slider tab 2502 may be coupled to wires 2420, 2430, and 2440 such that sliding slider tab 2502 away from shaft 2410 pulls wires 2420, 2430, and 2440 back into shaft 2410.

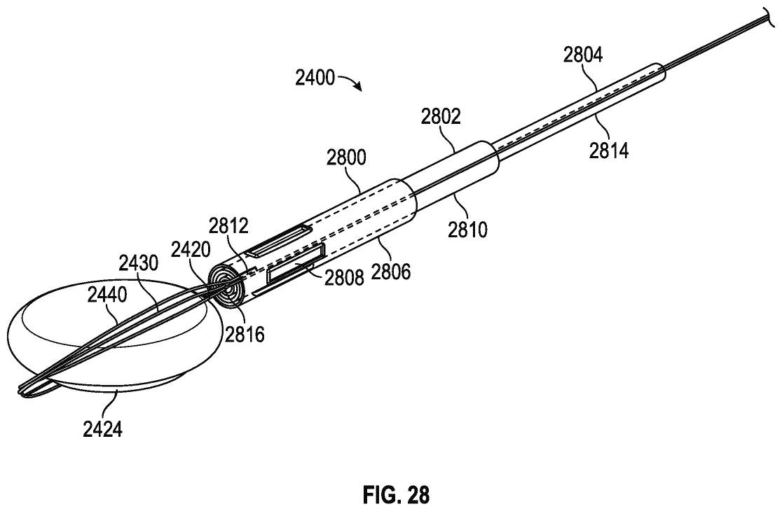

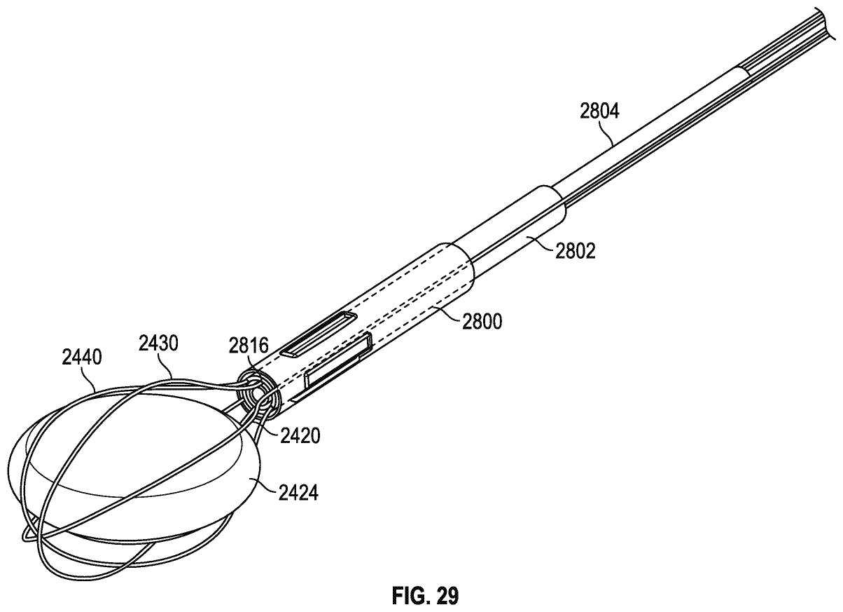

Although a single slider tab 2502 is shown in FIG. 26, it should be appreciated that two, three, or more sliders may be provided that slide in parallel within groove 2504 to move wires 2420, 2430, and 2440 and/or one or more separators or encapsulating bags individually or in groups. Actuation of sliders such as slider tab 2502 can control, together or separately, wires 2420, 2430, and 2440 and/or other components to deploy, retract, and/or rotate the wires and/or components attached thereto. According to some embodiments, the cross-section of the slider mechanism within groove 2504 (and coupled to slider tab 2502) can be various shapes including circular (e.g., one tube within another which allows for rotational motion in addition to translational motion, see FIGS. 28 and 29 as examples).

One or more slider tabs such as slider tab 2502 may each be attached to one or both ends of the wire loops that form wires 2420, 2430, and 2440. For example, one end of each wire loop may be fixed within shaft 2410 and an opposing end attached to a slider tab so that extending the wire from lumen 2413 is performed by moving one side of the wire out of the lumen while the other side remains fixed. As another example, both ends of each wire loop may be attached to a slider tab so that extending the wire from lumen 2413 is performed by moving the entire loop. Wires 2420, 2430, and 2440 may be manipulated around lens 2424 together, prior to separation, or may be sequentially manipulated around lens 2424 after separation.

FIG. 27 shows a perspective view of extraction device 2400 in which a control mechanism 2700 is provided at an end of handle 2500 opposite to the end at which delivery shaft 2410 is attached. In the example of FIG. 27, control mechanism 2700 includes a handle 2704 coupled to a threaded shaft 2702. In this implementation, handle 2704 may be rotated to move shaft 2410 (coupled to delivery shaft 2410 within handle 2500) to control the distance to which shaft 2410 extends from handle 2500. In some implementations, control mechanism 2700 may also allow for wires 2420, 2430, and 2440 to be removed from shaft 2410 and handle 2500 so that another tool such as a forceps can be inserted to remove cut portions of lens 2424 from the patient's eye via lumen 2413. In other implementations, a forceps may be included within delivery shaft 2410 and separately operable from wires 2420, 2430, and 2440. In still other implementations, forceps separate from extraction device 2400 may be used to extract lens pieces from the eye after the lens has been cut by wires 2420, 2430, and 2440. In other implementations, as discussed in further details hereinafter, additional elements such as a mesh or stent, and an encapsulation bag may be used in combination with wire loops 2420, 2430, and 2440 for extraction of lens pieces.

It should be appreciated that, although wires 2420, 2430, and 2440 are shown to be linearly separated in the implementation shown in FIGS. 24-27, this is merely illustrative and other implementations of wires 2420, 2430, and 2440 are possible. For example, FIGS. 28 and 29 show an implementation of extraction device 2400 in which wires 2420, 2430, and 2440 are rotatable relative to one another to surround and dissect lens 2424. FIGS. 28 and 29 show extraction device 2400 without delivery shaft 2410 for clarity.

As shown in FIG. 28, wires 2420, 2430, and 2440 may be extended around lens 2424 together in a flattened, substantially planar common loop. In the implementation of FIGS. 28 and 29, device 2400 is provided with wire-guide structures 2800, 2802, and 2804 (e.g., substantially cylindrical nested wire guide structures that are slidable and/or rotatable within delivery shaft 2410) configured to rotate wires 2420, 2430, and 2440 relative to each other to substantially surround lens 2424 (e.g., as in the configuration of FIG. 29).

Wire-guide structure 2800 may include a cylindrical main body 2806 (e.g., a portion of shaft 2410) and one or more protrusions 2808 (e.g., on the inner surface of shaft 2410 or on an outer surface of structure 2810) that guide the position of wire 2420. For example, a portion of wire 2420 may be positioned between two of protrusions 2808 and between the outer surface of cylindrical main body 2810 and the inner surface of delivery shaft 2410 so that the position of wire 2420 can be controlled by protrusions 2808.

Wire-guide structure 2802 may include a cylindrical main body 2810 that is at least partially nested within cylindrical main body 2806 of wire-guide structure 2800. Wire-guide structure 2802 may include a notch 2812 that allows wire 2440 to pass into an internal recess within wire-guide structure 2802 so that the rotational position of wire 2440 can be controlled by rotation of wire-guide structure 2802 (e.g., relative to wire-guide structures 2800 and 2804).

Wire-guide structure 2804 may include a cylindrical main body 2814 that is at least partially nested within cylindrical main body 2810 of wire-guide structure 2802. Wire-guide structure 2804 may include a notch 2816 that allows wire 2430 to pass into an internal recess within wire-guide structure 2804 so that the rotational position of wire 2430 can be controlled by rotation of wire-guide structure 2804 (e.g., relative to wire-guide structures 2800 and 2802).

In the implementations of FIGS. 28 and 29, first, second, and third wire loops 2420, 2440, and 2430 are configured to separate by a rotation of second and third wire loops 2440 and 2430 relative to first wire loop 2420. Wire-guide structures 2800, 2802, and 2804 may include first, second, and third nested wire-guide structures and configured to control a rotational position of first, second, and third wire loops 2420, 2440, and 2430. First wire-guide structure 2800 may include a cylindrical main body 2806 (e.g., a portion of shaft 2410) and a plurality of protrusions 2808 on an outer surface of the cylindrical main body 2810 or on an inner surface of shaft 2410. Second wire-guide structure 2802 may include a cylindrical main body 2810 at least partially nested within the cylindrical main body 2806 of first wire-guide structure 2800 (e.g., within shaft 2410) and at least one slot 2812 for the second wire loop 2440. Third wire-guide structure 2804 may include a cylindrical main body 2814 at least partially nested within the cylindrical main body 2810 of the second wire-guide structure and at least one slot 2816 for the third wire loop 2430. Second and third wire-guide structures 2802 and 2804 are rotatable relative to first wire-guide structure 2800. First, second, and third wire-guide structures 2800, 2802, and 2804 are slidable within delivery shaft 2410 for extension and retraction of the first, second, and third wire loops 2420, 2440, and 2430.

As shown in FIG. 29, after wires 2420, 2430, and 2440 have been deployed as a flat, somewhat circular, group of multiple wire loops (see, FIG. 28) wire-guide structures 2802 and 2804 may be rotated relative to wire-guide structure 2800 so that one or more wire loops 2420, 2430, and 2440 are rotated to surround the lens. Once in the configuration shown in FIG. 29, wires 2420, 2430, and 2440 can be retracted into delivery shaft 2410 to dissect the lens into multiple pieces as the wires are retracted. The dissected lens pieces can then be removed from the eye using forceps.

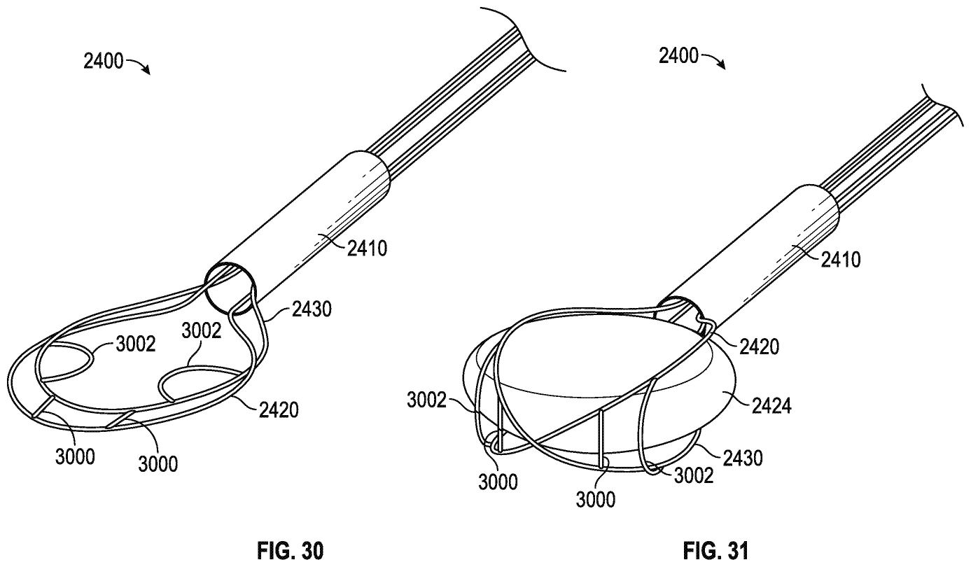

It should be appreciated that, although three wires are shown in the implementations of FIGS. 24-29, more or less than three wires may be provided that can be extended, separated, rotated, and/or retracted to divide lens 2424 into a desired number and size of pieces when the wires are retracted into lumen 2413. For example, FIGS. 30 and 31 show an implementation of extraction device 2400 in which two wires 2420 and 2430 are provided with interconnecting wires 3000 and 3002 that couple wires 2420 and 2430 at various locations around the respective wire loops. In the implementation of FIGS. 30 and 31, two relatively shorter interconnecting wires 3000 extend between wire loops 2420 and 2430 near a distal end of the loops and two relatively longer interconnecting wires 3002 extend between wire loops 2420 and 2430 nearer the distal end of delivery shaft 2410. By rotating wire loops 2420 and 2430 relative to one another, interconnecting wires 3000 and 3002 may be extended from a relaxed shape (as shown in FIG. 30) to a more straight and tensioned shape (as shown in FIG. 31), and thereby form a net-like structure which may surround lens 2424. First and second wire loops 2420 and 2430 in the implementation of FIGS. 30 and 31 are configured to separate by a rotation of first and/or second wire loops.

Wire-guide structures such as wire-guide structures 2800 and 2802 may be provided within delivery shaft 2410 to control the relative rotation of wire loops 2420 and 2430. Once in the configuration shown in FIG. 29, wires 2420 and 2430 and interconnecting wires 3000 and 3002 can be retracted into delivery shaft 2410 to dissect the lens into multiple pieces as the wires are retracted and wires 2420 and 2430 and interconnecting wires 3000 and 3002 cut through lens 2424. The dissected lens pieces can then be removed from the eye using forceps or other structures such as a wire mesh or stent and/or an encapsulation bag.

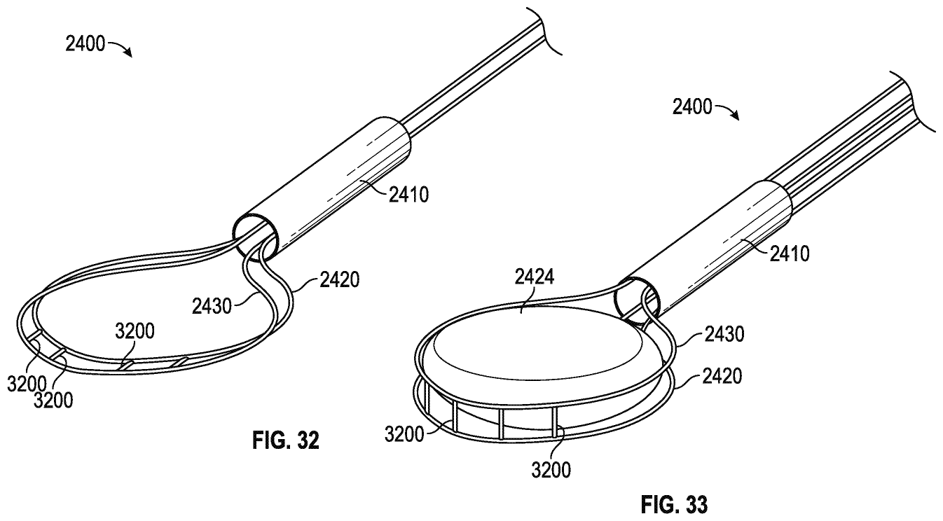

Although wires 2420 and 2430 are rotated in the implementation of FIGS. 30 and 31, it should be appreciated that one or more wire loops with interconnecting wires may be provided that separate to surround and dissect lens 2424. For example, FIGS. 32 and 33 show an implementation of extraction device 2400 in which two linearly separable wires 2420 and 2430 are provided with interconnecting wires 3000 that couple wires 2420 and 2430 at various locations around the respective wire loops. In the implementation of FIGS. 32 and 33, interconnecting wires 3000 of substantially equal length extend between wire loops 2420 and 2430 at various locations around the loops. By separating wire loops 2420 and 2430 after extension, interconnecting wires 3000 may be extended from a relaxed shape (as shown in FIG. 32) to a more straight and tensioned shape (as shown in FIG. 33), and thereby form a net-like structure which may surround lens 2424. First and second wire loops 2420 and 2430 in the configuration of FIGS. 31 and 32 may be configured to linearly separate to a separated configuration in which first wire loop 2420 is substantially parallel to second wire loop 2430 or in which wire loops 2420 and 2430 are separated by an increasing distance with increasing distance from shaft 2410 (see, e.g., FIG. 24).

Once in the configuration shown in FIG. 33, wires 2420 and 2430 and interconnecting wires 3000 can be retracted into delivery shaft 2410 to dissect the lens into multiple pieces as the wires are retracted and wires 2420 and 2430 and interconnecting wires 3000 cut through lens 2424. The dissected lens pieces can then be removed from the eye using forceps or other components such as a wire mesh and/or an encapsulation bag.

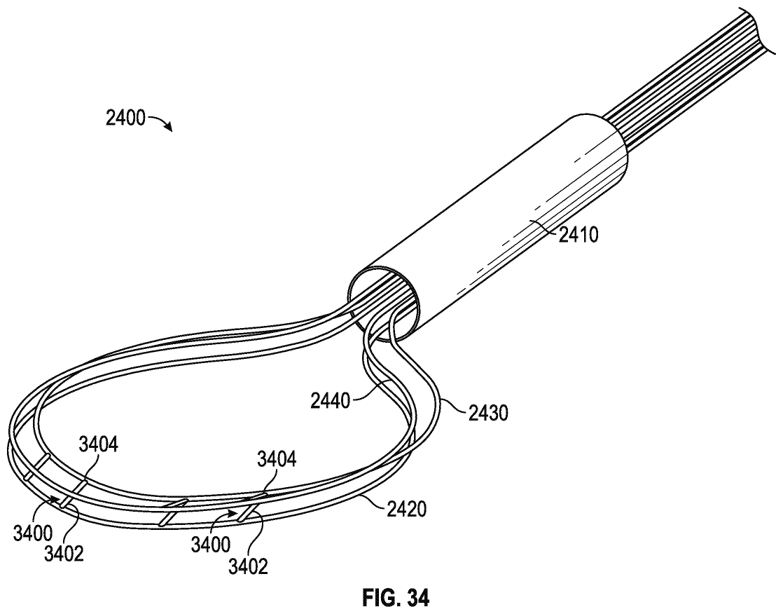

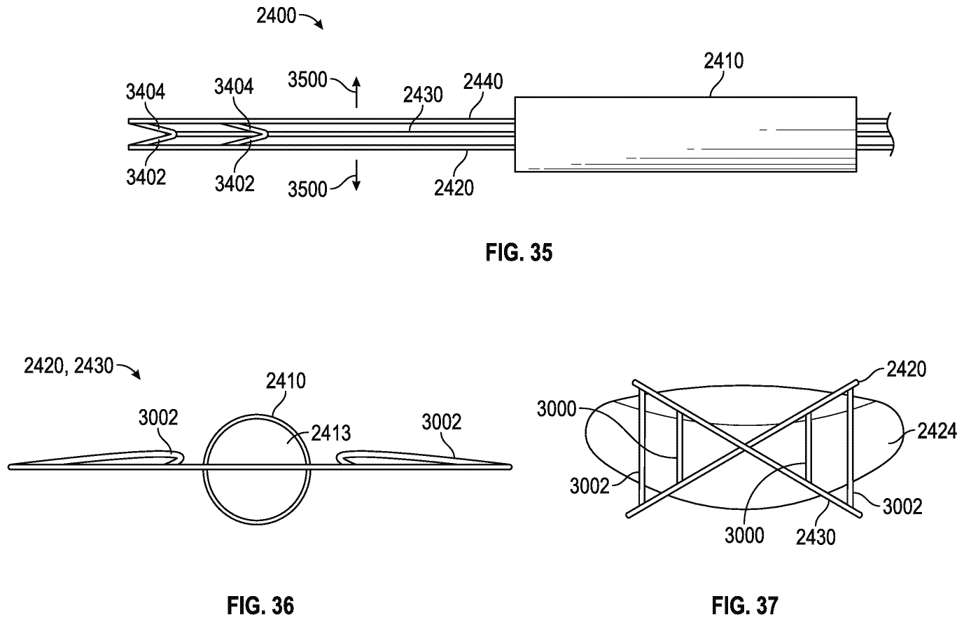

As another example, FIGS. 34 and 35 show an implementation of extraction device 2400 in which three linearly separable wires 2420, 2430, and 2440 are provided with interconnecting wires 3400 that couple wires 2420, 2430, and 2440 at various locations around the respective wire loops. In the implementation of FIGS. 34 and 35, interconnecting wires 3400 of substantially equal length extend between wire loops 2420, 2430, and 2440 at various locations around the loops. Each interconnecting wire includes a first portion 3402 that extends from wire 2420 to wire 2430 and a second portion 3404 that extends from wire 2430 to wire 2440. In the example of FIGS. 34, and 35, portions 3402 and 3404 are located at the same location on the loops. However, portions 3402 and 3404 may be located at different locations around the loops in some implementations.

FIG. 35 shows a side view of the implementation of device 2400 of FIG. 34 in which it can be seen that, prior to separation of wire loops 2420, 2430, and 2440, middle loop 2430 is initially deployed from delivery shaft 2410 with a smaller diameter than the top and bottom loops 2420 and 2430, which causes the group of loops 2420, 2430, and 2440 to lay flat by tensioning the interconnecting wires 3400, and thus pulling the top and bottom loops 2420 and 2440 toward the middle loop 2430. Middle loop 2430 can then be pushed outward from delivery shaft 2410, such that the diameter of middle loop 2430 is increased to the same diameter as that of top and bottom loops 2420 and 2430, causing interconnecting wires 3400 to straighten, pushing wire loops 2420 and 2440 apart (e.g., as indicated by arrows 3500). In the flattened configuration of FIGS. 34 and 35, wire loops 2420, 2430, and 2440 can be moved to surround lens 2424. Wire loops 2420, 2430, and 2440 can then be expanded or separated by extending middle loop 2430 as described, such that interconnecting wires 3400 stand roughly perpendicular to the main loops 2420, 2430, and 2440. Wire loops 2420, 2430, and 2440 can then be retracted into delivery shaft 2410 to cut the lens into multiple pieces as wires 2420, 2430, and 2440 and interconnecting wires 3400 pass through the lens. The dissected lens pieces can then be removed from the eye using forceps or other components such as a wire mesh and/or an encapsulation bag.

For further clarity, face-on views of various implementations of extraction device 2400 (e.g. views facing the distalmost end of device 2400 along the central longitudinal axis of device 2400) are shown in FIGS. 36-40. More specifically, FIGS. 36 and 37 show face-on views of device 2400 in the implementation of FIGS. 30 and 31, FIGS. 38 and 39 show face-on views of device 2400 in the implementation of FIGS. 32 and 33, and FIG. 40 shows a face-on view of device 2400 in the implementation of FIGS. 34 and 35.

FIG. 36 shows a face-on view of device 2400 in the implementation and in the flattened configuration of FIG. 30, and shows how interconnecting wires 3002 may be curved slightly out of a plane defined by non-rotated wires 2420 and 2430 in the flattened configuration. FIG. 37 shows a face-on view of device 2400 in the implementation and rotated configuration of FIG. 31 and shows how interconnecting wires 3000 and 3002 may be straightened by rotated wires 2420 and 2430 that form an x-pattern in the rotated configuration.

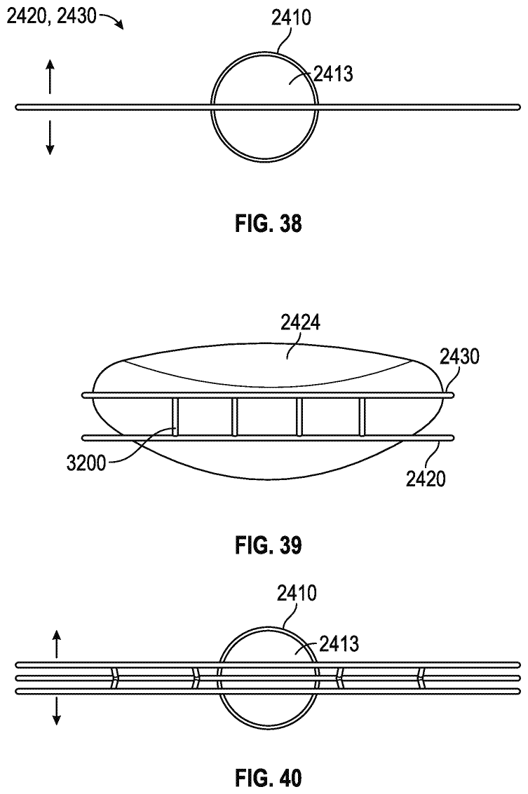

FIG. 38 shows a face-on view of device 2400 in the implementation and flattened-configuration of FIG. 32. By separating wires 2420 and 2430 as indicated by arrows 3800, interconnecting wires 3200 may be straightened so that, as shown in FIG. 39, wires 2420 and 2430 may be substantially parallel with substantially perpendicular interconnecting wires 3200 interposed between.

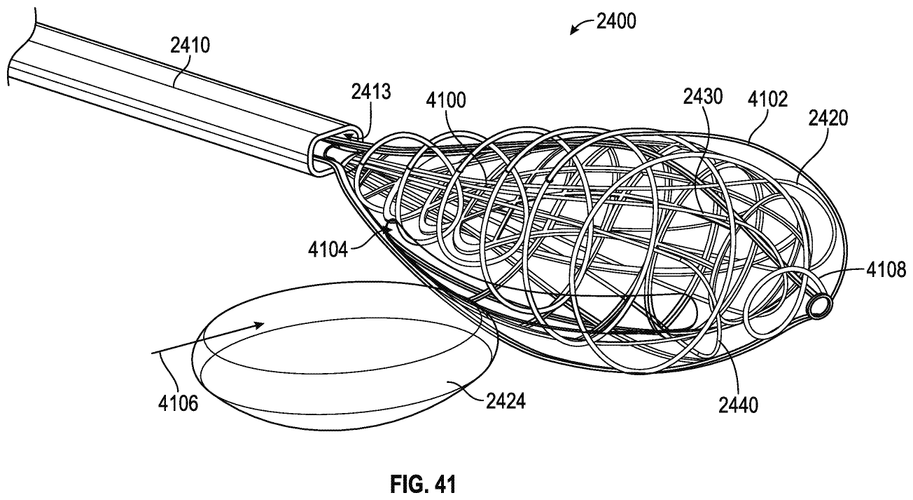

FIG. 40 shows a face-on view of device 2400 in the implementation and flattened-configuration of FIGS. 34 and 35. By separating wires 2420, 2430, and 2440 (e.g., by extending middle wire 2430 as described above in connection with FIGS. 34 and 35) as indicated by arrows 4000, interconnecting wires 3400 may be straightened wires 2420, 2430, and 2440 may be substantially parallel with substantially perpendicular interconnecting wires 3400 interposed between.

The various implementations of extraction device 2400 described in connection with FIGS. 24-40 may be used to dissect lens 2424 into pieces (e.g., roughly equal sized pieces in the example of FIGS. 34, 35, and 40) that individually are sized to fit down delivery shaft 2410 for extraction. However, it should also be appreciated that extraction device 2400 may include one or more other components, in addition to one, two, three, or more cutting wires, such as a wire mesh or stent (e.g., to further dissect the cut lens pieces) and/or an encapsulation bag, extendible over the wires and/or the stent to encapsulate the cut/dissected lens for extraction through delivery shaft 2410. In some implementations, the wire mesh or stent can be used in place of, or in addition to, the cutting wires as described in connection with the implementations shown FIGS. 24-40.

FIG. 41 shows an exemplary implementation of extraction device 2400 in which a stent 4100 and an encapsulation bag 4102 are provided in addition to wire loops 2420, 2430, and 2440. As shown in FIG. 41, after extension of wires 2420, 2430, and 2440, a wire mesh or stent such as stent 4100 may be extended around wire loops 2420, 2430, and 2440. Encapsulation bag 4102 may be extended around stent 4100 (e.g., as described above in connection with various implementations). An opening 4104 may be provided (e.g., an opening formed by coaligned openings in bag 4102 and stent 4100) through which lens 2424 can be inserted into a cavity formed and defined by stent 4100 and bag 4102. For example, by moving extraction device 2400 toward lens 2424 so that lens 2424 (while remaining substantially stationary within the patient's eye) is moved in direction 4106 relative to device 2400, lens 2424 can be inserted through opening 4104 into the cavity and substantially surrounded by wires 2420, 2430, and 2440, stent 4100, and bag 4102.

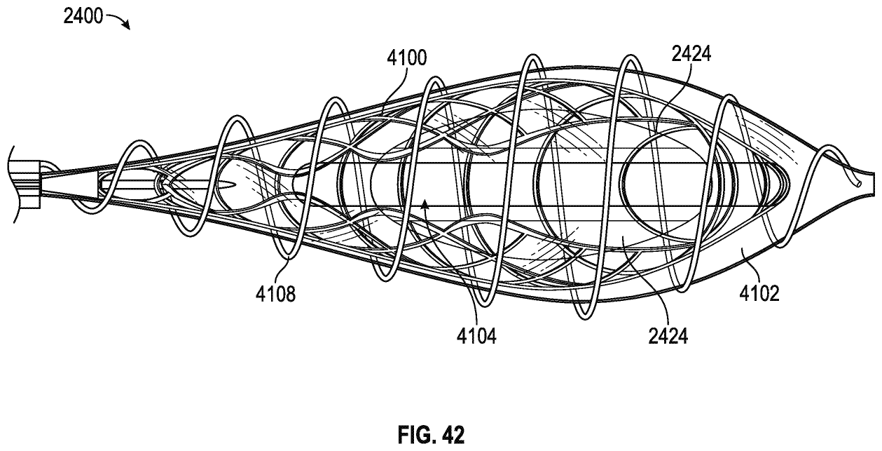

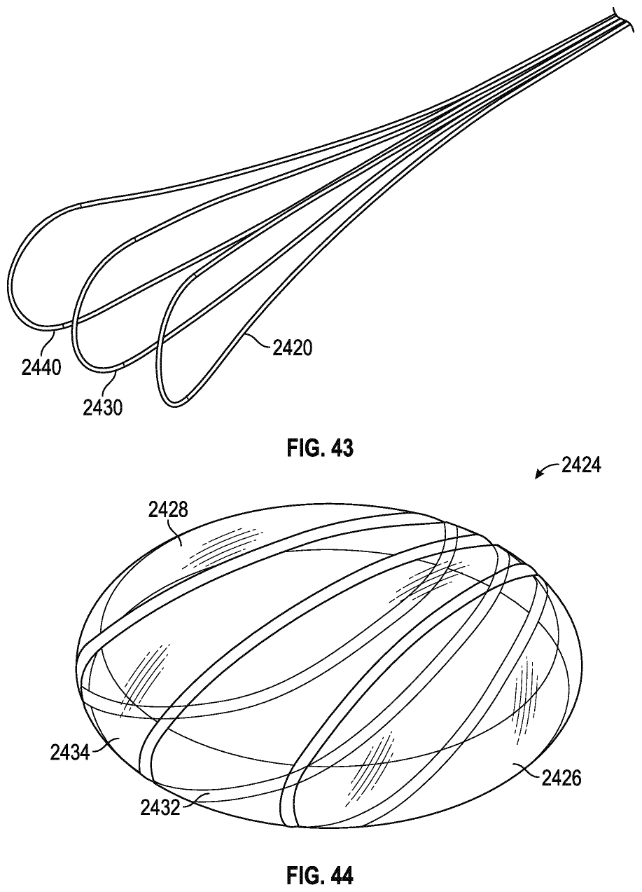

FIG. 42 shows a side view of extraction device 2400 in the configuration of FIG. 41 after insertion of lens 2424 into the cavity formed by wires 2420, 2430, and 2440, stent 4100, and bag 4102. Following insertion of the lens into the cavity formed by wires 2420, 2430, and 2440, stent 4100, and bag 4102, wires 2420, 2430, and 2440 may be retracted into lumen 2413 to cut lens 2424 (e.g., as described above in connection with FIGS. 24 and 25), and then stent 4100 may be retracted to further cut and/or cube the lens into smaller, softer pieces.

Device 2400 may also include a spiral compaction wire 4108 wrapped in spiral windings around bag 4102. After wires 2420, 2430, and 2440 and stent 4100 have been retracted to cut and cube lens 2424 within bag 4102, spiral compaction wire 4108 may be retracted to further compact the pieces of lens 2424 within encapsulation bag 4102 for extraction from the eye (e.g., by extracting bag 4102 and wire 4108 into lumen 2413 or by withdrawing device 2400 from the eye without extracting bag 4102 back into lumen 2413).

FIG. 43 shows wires 2420, 2430, and 2440 of device 2400 of FIG. 41 with other components of device 2400 removed for clarity. Wires 2420, 2430, and 2440 may be formed, for example, from nitinol, may have a cross-sectional width of, for example, 0.004″, and may be extended and/or withdrawn from lumen 2413 as described above in connection with, for example FIGS. 24-27. FIG. 44 shows an illustrative example of a lens 2424 that has been cut into pieces 2426, 2428, 2432, and 2434 by retraction of wires 2420, 2430, and 2440 through lens 2424 into lumen 2413.

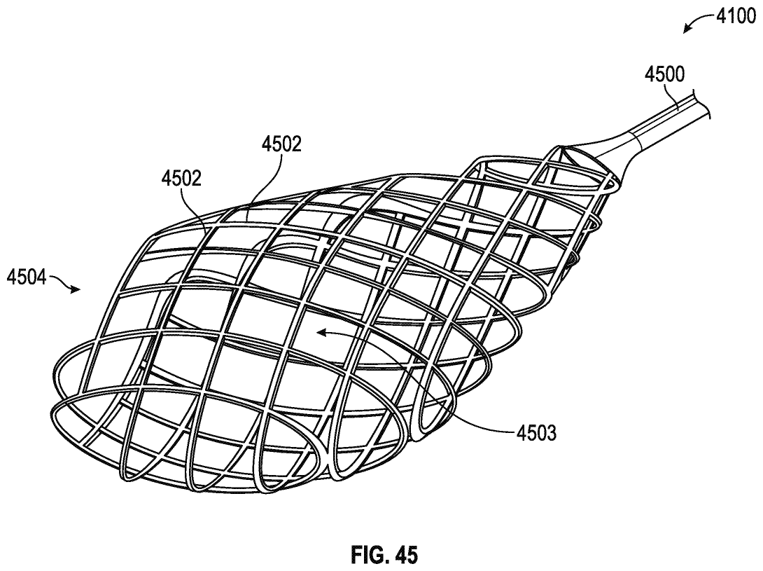

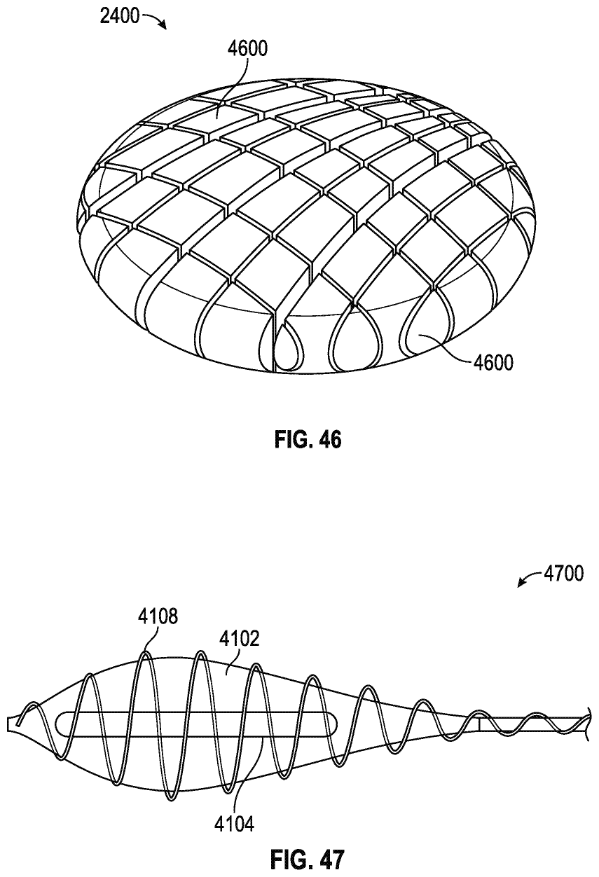

FIG. 45 shows stent 4100 of device 2400 of FIG. 41 with other components of device 2400 removed for clarity. Stent 4100 may include a plurality of intersecting wire elements 4502 extending from a shaft 4500 that form and define a cavity 4503. Stent 4100 may be formed from a wire mesh braid or may be a laser-cut structure. Intersecting wire elements 4502 may be formed from metal and/or plastic and may have a cross-sectional width of between 0.001″ and 0.005″ (e.g., 0.003 inches) in some implementations. Intersecting wire elements 4502 may be configured to return to a natural expanded shape, as shown in FIG. 45, when extended from within lumen 2413 (e.g., from a compressed shape within lumen 2413) to form a cavity 4503, accessible by lens 2424 through an opening 4504. FIG. 46 shows an illustrative example of a lens 2424 in which pieces 2426, 2428, 2432, and 2434 have been cut/cubed into smaller pieces 4600 by retraction and resulting compression of stent 4100 through lens 2424 into lumen 2413.

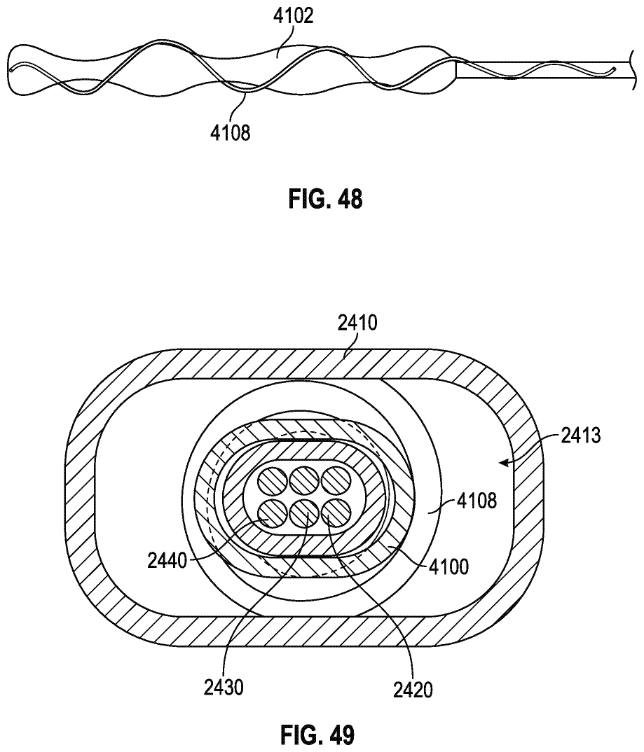

FIG. 47 shows encapsulation bag 4102 and spiral compaction wire 4108 of device 2400 of FIG. 41 with other components of device 2400 removed for clarity. Encapsulation bag 4102 may be formed from an elastic material such as an expanded polytetrafluoroethylene (ePTFE) material. Encapsulation bag 4102 may include an opening 4704. Opening 4704 may be configured to be coaligned with opening 4504 of stent 4100, when stent 4100 and bag 4102 are in an extended configuration, to form opening 4104 of FIG. 41. Spiral compaction wire 4108 may be formed from a metal such as nitinol and may have a cross-sectional width of, for example, between, 0.001″ and 0.005″ (e.g., 0.003″). Spiral compaction wire 4108 may be extracted to radially compress encapsulation bag 4102, in a circular motion, into the configuration of FIG. 48, to collect and compress pieces 4600 of lens 2424 for extraction from the eye. Encapsulation bag 4102 can be removed from the patient's eye by retraction into lumen 2413 or by extraction of the entire device 2400 from the eye without pulling bag 4102 back into lumen 2413.

FIG. 49 shows a cross-sectional view of delivery shaft 2410 with wires 2420, 2430, and 2440, stent 4100, and spiral compaction wire 4108 disposed within lumen 2413 (e.g., prior to extension from, or after retraction into lumen 2413). For clarity, encapsulation bag 4102 is not shown in FIG. 49, but would occupy the empty space visible in lumen 2413. As shown in FIG. 49, in some implementations delivery shaft 2410 may have a cross-sectional shape other than a circular cross-sectional shape (e.g., a rectangular cross-sectional shape with rounded corners). After retraction of wires 2420, 2430, and 2440 and stent 4100 through lens 2424 into lumen 2413, some pieces of lens 2424 may also occupy some of the space within lumen 2413 (e.g., by being pulled into delivery shaft 2410 during the retraction).

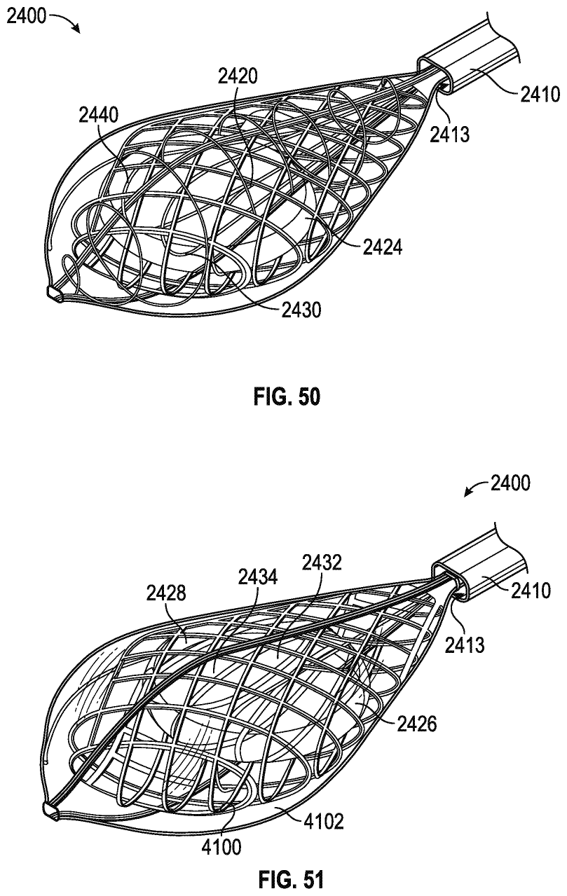

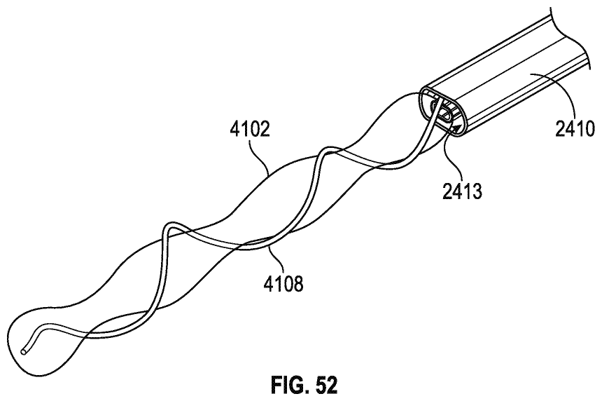

FIGS. 50-52 illustrate extraction device 2400 of FIG. 41 at various stages during cataract extraction operations. In the example of FIG. 50, lens 2424 is disposed within the cavity formed by wires 2420, 2430, and 2440, stent 4100, and encapsulation bag 4102 and wires 2420, 2430, and 2440 have been partially retracted into lumen 2413 and have partially passed into lens 2424. In the example of FIG. 51, lens 2424 has been cut into pieces 2426, 2428, 2432, and 2434 and wires 2420, 2430, and 2440 have been retracted into lumen 2413. Stent 4100 and bag 4102 remain surrounding lens 2424. In the example of FIG. 52, stent 4100 has been retracted into lumen 2413 and spiral compaction wire has been retracted to radially compress bag 4102 to encapsulate, capture, and contain any remaining pieces of lens 2424. In the configuration of FIG. 52, bag 4102 can be retracted into lumen 2413 or device 2400 can be entirely removed from the patient's eye without retracting bag 4102.

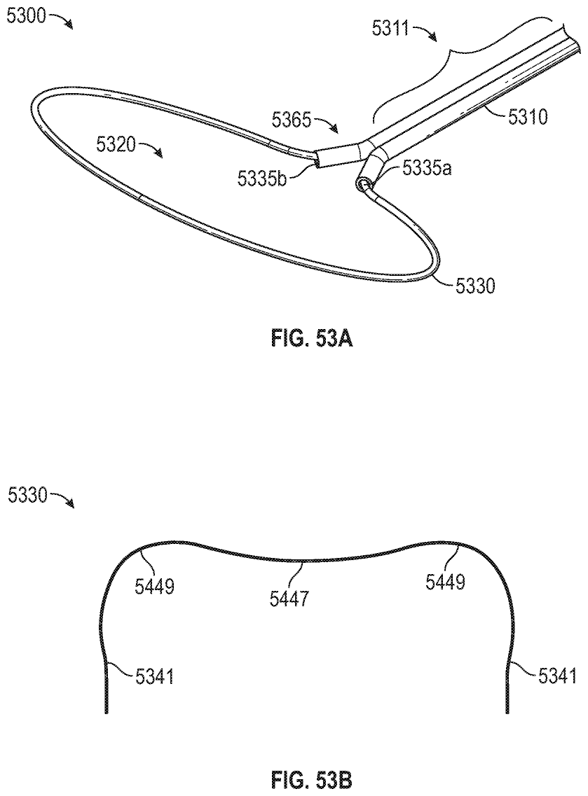

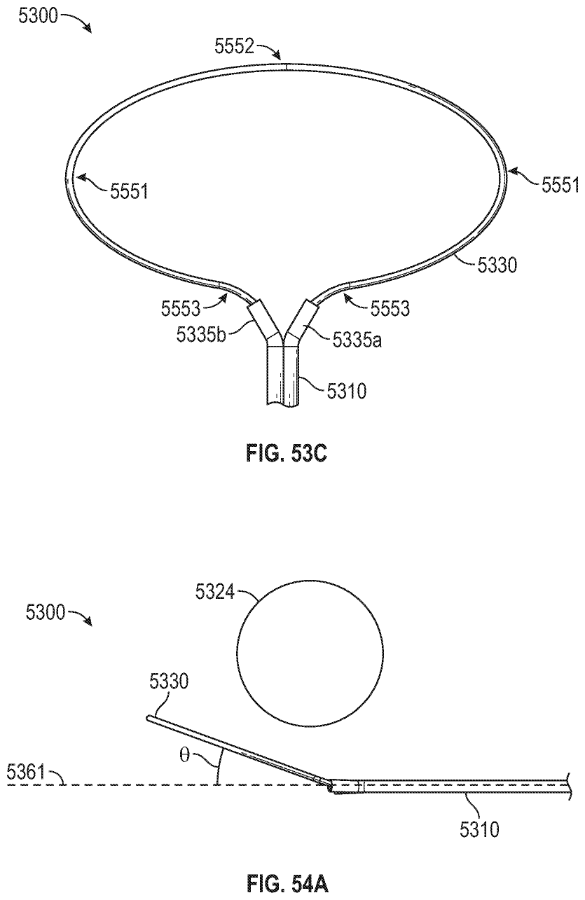

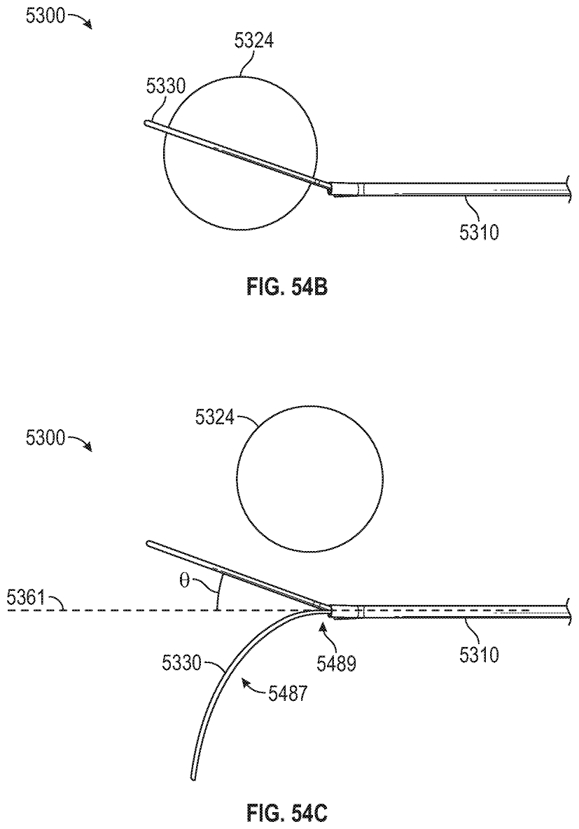

According to some embodiments, for example as shown in FIG. 53A, an extraction device 5300 may include a delivery shaft 5310 having a lumen and a distal end. The lumen may include multiple separated channels for accommodating portions of wire therein. For example, as shown in FIG. 53A, the lumen at the distal end of the delivery shaft 5310 may include a first channel 5335a and a second channel 5335b diverging relative to each other for accommodating ends or portions of a wire 5330. Embodiments are also contemplated in which the separate first and second channels 5335a and 5335b need not diverge from one another (for example, they may extend parallel to each other). The lumen may extend through branching arms 5365 disposed at a distal end of the delivery shaft 5310. According to some embodiments, the wire 5330 may provide a wire loop formed from metal (e.g., nitinol), plastic, and/or a shape memory material. According to some embodiments, as shown in FIG. 53A, the pair of distinct channels 5335a and 5335b may extend an entire length of the delivery shaft 5310. In such embodiments, separate channels at the proximal region 5311 of the delivery shaft 5310 respectively accommodate the two ends of the wire 5330, which may facilitate smooth sliding engagement of the wire 5330 within the lumen. Alternatively, according to some embodiments, the distinct channels 5335a and 5335b may diverge distally from a single channel of the delivery shaft 5310. In such embodiments, all or a portion of proximal region 5311 of the delivery shaft may include a single channel that accommodates both ends of the wire 5330. The distal end of the delivery shaft 5310 and/or the channels 5335a and 5335b may be designed to direct the wire 5330 to form a loop either in a plane with the longitudinal axis of the delivery shaft 5310 or out of plane with the longitudinal axis of the delivery shaft 5310 as shown in FIGS. 53A and 54B.

The wire 5330 may be slidable or otherwise movable relative to the delivery shaft to allow for extension or withdrawal of the wire loop, as desired. According to some embodiments, the wire 5330 may be actuated by a manually or automatically controlled mechanism based on mechanical or electronic activation. The divergence of the channels 5335a and 5335b may facilitate expansion and contraction of a diameter of the wire loop with sliding engagement of the wire 5330 relative to the delivery shaft. A capture region 5320 can be defined, at least in part, by the loop of the wire 5330. Extension (deployment) of the wire 5330 in a distal direction may increase the diameter of the wire loop, causing the size of the capture region 5320 to expand. Retraction (withdrawal) of the wire 5330 in a proximal direction may decrease the diameter of the wire loop, causing the size of the capture region 5320 to shrink.

According to some embodiments, the wire 5330 may be shape set so that it has a preformed, natural shape that forms a closed loop. For example, the wire 5330 may be made from a nitinol wire or shape memory material that has a natural shape forming a closed loop like that shown in FIG. 53A. Such a natural shape can allow the wire to form capture region 5320 without a need for any external bias or force applied by the delivery shaft 5310 when the wire 5320 is in a fully extended or deployed position.

According to some embodiments, the wire 5330 may be a freely flexible wire that is not shape set to form any well-defined natural shape. For example, the wire 5330 may be made from a freely flexible material in which the delivery shaft 5310 or other components of the extraction device 5300 are entirely responsible for constraining or orienting portions of the wire 5330 to form a desired shape such as the closed loop shown in FIG. 53A.

According to some embodiments, for example as shown in FIG. 53B, the wire 5330 may be shape set so that it has a preformed, natural shape other than the shape formed by the wire 5330 when the wire is in the fully deployed or extended position in the extraction device 5300. In such embodiments, components of the extraction device 5300 such as the delivery shaft 5310 can be used to constrain or hold end portions of the wire together so that the wire 5330 forms a closed loop defining capture region 5320 in the fully extended position. FIG. 53B shows an example of such a wire in its natural state, in the absence of any external force or bias.

As seen in FIG. 53B, the wire 5330 may be shape set to form a preformed, natural shape that is an open shape larger than the shape formed by the wire when the wire is in the fully extended position. Such an open shape can be a non-linear shape, such as a generally U-shaped structure or another curved or bent shape that does not form a closed loop in the absence of any external force. The wire 5330 can have preformed bent regions or curved regions 5449 along its length to facilitate formation of the non-linear shape. The preformed bent regions 5449 may include a pair of preformed bent regions located on opposing sides of an intervening region 5447. In the natural state, the intervening region 5447 may be straight, may have lower degree of curvature (larger radius) than the preformed bent regions 5449, or may have an oppositely oriented bend relative to the preformed bent regions 5449.

The wire 5330 can be formed into a loop in the device by constraining end portions 5341 of the wire 5330 within the delivery shaft, as shown for example in FIG. 53C (e.g., by constricting the end portions 5341 towards each other within the channels 5335a and 5335b). According to some embodiments, the preformed curved regions 5449 and intervening region 5447 can be used to urge the wire 5330 into an oblong lenticular shape (e.g., a generally elliptical shape) when the end portions 5341 are constrained by the delivery shaft 5310. The oblong shape can have a long axis (or “major axis”) and a short axis (or “minor axis”). As seen in the figures, the long axis can be oriented transverse to the axis of the delivery shaft (e.g., perpendicular to the longitudinal shaft axis or otherwise transverse). More particularly, the loop can be oriented such that the lateral dimension of the loop (dimension measured perpendicular to the shaft axis) is greater than the longitudinal dimension of the loop (dimension measuring parallel to or along the same direction as the shaft axis). Such an orientation can facilitate efficient capture or encircling of the lens with the wire loop, for example by bending or tilting the delivery shaft to move the deployed wire loop down and around the lens upon entry into the anterior chamber. With reference to FIGS. 53B-C, the preformed curved regions 5449 can correspond to regions of high curvature 5551 (or smaller local radius of the loop) for the lenticular shape, where the loop may otherwise have a tendency to form a circular loop in the absence of the preformed curved regions 5449. Such a lenticular shape can in turn facilitate capture or encircling of a lens while the lens is in the eye with greater precision and/or without adding undue trauma to other structures of the eye surrounding the lens. Additionally or alternatively, in some embodiments the naturally open preformed shape of the wire may create forces on the wire that serve to maintain structural stability of the wire when the wire is constrained to form a closed loop extending from the delivery shaft in the fully extended position. For example, the end portions 5335 of the wire 5330 may provide an outward force against the delivery shaft 5310, and the delivery shaft 5310 may provide an inward counterforce against the wire, as the preformed shape of the wire urges the wire to further expand to return to its natural shape shown in FIG. 53B. Such forces may in turn eliminate slack that may otherwise be present in the wire 5330 and cause the wire to deform out of plane when deployed.

According to some embodiments, the wire may have a constant cross-section along its entire length. According to other embodiments, the wire may have a cross-section that varies along its length. The varying cross-section may vary in at least one of shape or area. Such variations in cross-section may be designed to achieve certain beneficial effects. For example, the cross-section may vary based on curvature of the wire loop (e.g., to facilitate a lenticular shape), to enhance cutting ability of the wire, to mitigate kinking, and/or to enhance structural stability of the deployed wire loop.

According to some embodiments, the wire may have a cross-section that varies based on the degree of curvature of the wire 5330 at a particular location along the length of the wire. Varying the cross-section in accordance with the degree of curvature may, for example, facilitate formation of a substantially lenticular shape for the wire loop when the wire loop is in the deployed, expanded configuration. FIG. 53C shows an example of a lenticular shape for the wire loop, and accordingly a lenticular shape for the capture region 5320. The distalmost region 5552 of the wire 5330 may have a lower degree of curvature than lateral regions 5551, and accordingly, the distalmost region 5552 may have a greater cross-sectional area than the lateral regions 5551 so as to provide a greater bending resistance at the distalmost region that facilitates such a shape. Alternatively, or in combination, the higher curvature lateral regions 5551 may have a different shape than the lower curvature distalmost region 5552. For example, the lateral regions 5551 may have an oblong cross-sectional shape while distalmost region 5552 may have a circular or equilateral cross-sectional shape. Some examples of oblong cross-sectional shapes an oval, a non-circular ellipse, and a non-square rectangle. In some embodiments, the oblong-shaped portion of the wire may be made flat, like a ribbon. An oblong shape generally has a long axis (e.g., axis running parallel to the longer sides of a rectangle) and a short axis (e.g., axis running parallel to the shorter sides of a rectangle). The oblong cross-sectional shape may be oriented for decreased bending resistance in-plane and increased bending resistance out-of-plane with respect to the plane of the loop. In such embodiments, the long axis of the oblong cross-section may be oriented perpendicular to or otherwise transverse to the plane of the loop, and the short axis may be oriented parallel to or along the plane of the loop.

In the example shown in FIG. 53C, the distalmost region 5552 is provided with lower curvature than the lateral regions 5551. However, it is contemplated that the locations along the length of the wire that are chosen for high and low curvature can be substantially anywhere, depending on the desired configuration or ergonomic considerations.

According to some embodiments, varying the cross-section between alternating shapes such as alternating round regions, which may be more conducive to bending or curving, and flat regions, which may be less conducive to bending or curving, may allow specific regions to either bend or not bend when the wire is extended. It is contemplated that such alternating cross-sectional shapes can be useful for cataract surgery or for other procedures, where it is desirable to steer a tube or wire across turns and not just straight. For example, the round segment can make a turn from one vessel branch to another for vascular surgery, or the round segment can urge a loop to bend into the capsule, while the straight segment can urge portions of the wire or the loop to go straight.

According to some embodiments, the cross-section may vary based on desired cutting ability. Such variations may involve smaller areas and/or sharper cross-sectional shapes (e.g., rectangular, square, or triangular) in regions of the wire where enhanced cutting ability is desired. For example with reference to FIGS. 53C, the distalmost region 5552 of the wire 5330 may have a smaller cross-sectional area than lateral regions 5551 so as to enhance a cutting ability of the wire at the distalmost region as the distalmost region is retracted against the lens. Additionally or alternatively, the distalmost region 5552 may have a sharper or more pointed shape (e.g., triangular, rectangular, or square) compared to lateral regions 5551. In this example, the distalmost region 5552 is provided with enhanced cutting ability. However, it is contemplated that the locations along the length of the wire that are chosen for enhanced cutting ability can be substantially any location along the wire where enhanced cutting ability is desired.

According to some embodiments, the cross-section may vary based on regions of stress in the wire to mitigate kinking. For example, the exit regions 5553 of the wire 5330 can be susceptible to kinking due to a sharp bend or small radius curvature upon shaft exit. The exit regions 5553 of the wire can correspond to locations where the wire exits the delivery shaft 5310 and is adjacent to portions of the delivery shaft in the fully extended position. According to some embodiments, such regions can have a larger cross-sectional area or diameter than regions of the wire adjacent to the exit regions 5553 or other remaining regions of the wire 5330. For example, with reference to FIG. 53C, the exit regions 5553 and the distalmost region 5552 may have a square cross-section while other regions may have a round cross-section to provide alternating square and round regions (e.g., alternating every 0.05″ length of the wire loop 5330). Here, the intersection of the loop curvature will have a square cross-section.

According to some embodiments, the cross-section may vary to provide structural stability of the wire. For example, the exit regions 5553 of the wire 5330 may have an oblong cross-sectional shape with a long axis oriented transverse to the plane of the loop. Such a cross-sectional shape at the exit regions 5553 may provide structural stability by minimizing a tendency for the extended loop to bend out-of-plane, in a direction perpendicular or otherwise transverse to the plane of the loop, while permitting the wire to bend in-plane, in a direction along the plane of the loop. Alternatively, or in combination, the exit regions 5553 of the wire may have increased area relative to other regions of the wire to provide for enhanced structural stability for the extended loop.

According to some embodiments, for example, as shown in FIGS. 54A-54B, the capture region 5320 may be suitable for capturing or encircling a lens or other specimen like a lasso. FIG. 54A shows a side profile view of the extraction device 5300 and a lens 5324. FIG. 54B shows a side profile of the extraction device 5300 interacting with a specimen, e.g., by lassoing the lens 5324 with the wire loop to encircle the lens and capture it. The extraction device 5300 shown in FIGS. 54A-54B includes a bend angle θ of the wire loop relative to an axis 5361 (e.g., cylindrical axis) of the delivery shaft 5310. According to some embodiments, the bend angle θ may be implemented as an approximately 20 degree angle between the entire wire loop and the axis 5361. It will be appreciated that having the wire loop bent off-axis by 20 degrees in this manner is equivalent to forming an obtuse angle of 160 degrees between the entire wire loop and the delivery shaft 5310 itself. According to some embodiments, the bend angle θ may be implemented as a non-zero angle within a range of 0-180 degrees, and more particularly, within a range of 10-30 degrees.

According to some embodiments, the deployed wire 5330 may be arranged in a single plane prior to deployment, during deployment, and upon full deployment from the delivery shaft. According to some embodiments, the wire may be arranged in multiple planes prior to deployment, during deployment, and/or upon full deployment from the delivery shaft. For example, as shown in FIGS. 54A-54B, the deployed wire loop may be arranged along a plane to form a two-dimensional shape. As another example, as shown in FIG. 54C, a wire 5330 may not only bend at an angle 5489 from the shaft, but the wire 5330 may also be bent out of plane within the loop so that it forms an angle 5487 in other planes within the loop. Accordingly, the wire loop may form a three-dimensional shape such as that shown in FIG. 54C. Portions of the wire proximal to the loop and/or portions of the wire within the loop may be bent vertically or in plane of the loop, horizontally or out of the plane of the loop, or both vertically and horizontally. The wire proximal to and/or within the loop may be bent in such a manner that the angle increases progressively away from the shaft axis 5361 in a distal direction.

According to some embodiments, the wire 5330 is configured to extend from the delivery shaft straight, without forming any bend angle θ relative to the axis of the delivery shaft 5310.

According to some embodiments, the wire 5330 may be configured to vibrate in and/or out of the lumen. For example, the extraction device 5300 may be configured to induce vibration in the wire 5330 in the X, Y, and/or Z-plane to induce it to slide between tissue planes of the sample, such as between a lens and a capsular bag of a patient.

According to some embodiments, the wire 5330 may be colored or color-coded to enhance visualization once positioned in the eye. For example, the wire 5330 may be yellow, red, blue, or multicolored.

While one wire 5330 is pictured, in some embodiments paired wires, or more generally multiple wires may be utilized. The paired or multiple wires may each have the same size and shape, or the paired or distinct wires may differ in one or more of a size and shape when fully deployed from the lumen.

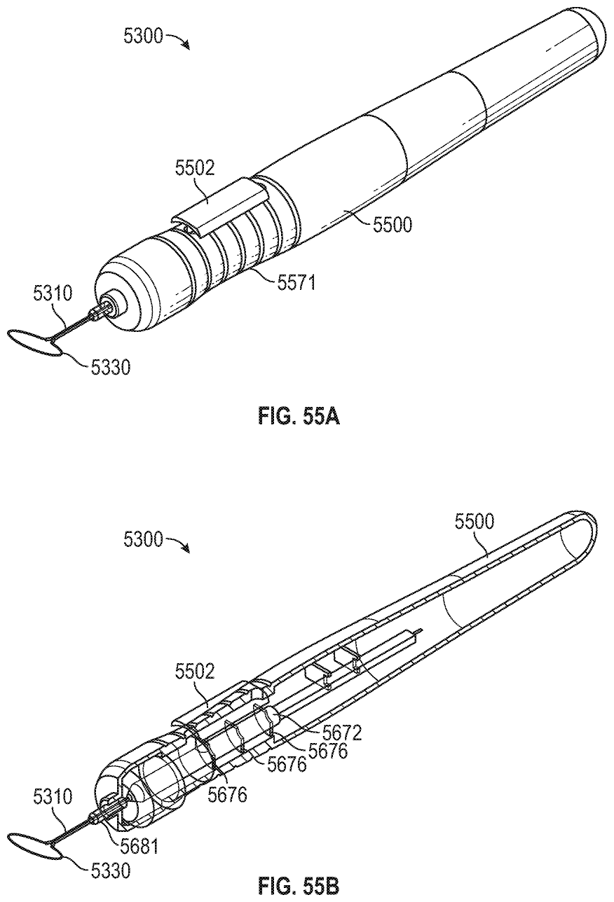

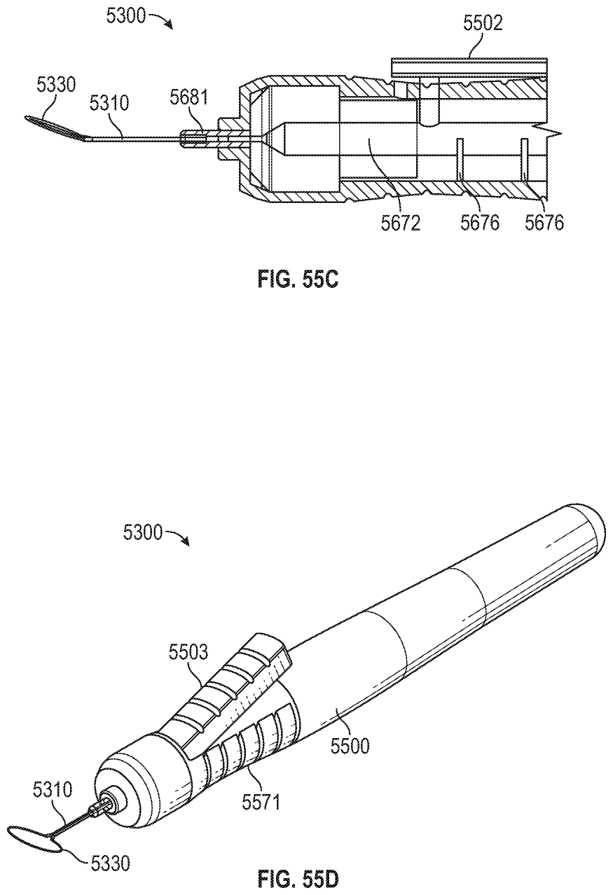

FIG. 55A shows a broader view of extraction device 5300 in which delivery shaft 5310 can be seen coupled to and extending from a handle 5500 of device 5300. As shown, delivery shaft 5310 may be a substantially cylindrical tube attached to handle 5500, which may also be implemented as a substantially cylindrical tube. According to some embodiments, for example, as shown in FIG. 55A, a proximal end of the delivery shaft 5310 may be attached to a distal end of the handle 5500. As shown, an external surface of the handle 5500 may include a grip 5571, which may include surface features (e.g., annular rings) and a contoured shape to facilitate manipulation of the extraction device like a surgical instrument.

According to some embodiments, the bend angle θ may additionally or alternatively be provided in the lumen of the delivery shaft 5310. In such embodiments, the wire 5330 may or may not have the bend angle noted previously, and the bend angle of the lumen may be provided before or after the transition to the diverging channels or branching arms of the lumen. In such embodiments, the bend angle θ may be a non-zero angle relative to an axis of the handle 5500, and may be within any of the ranges for the bend angle θ mentioned above. The bend angle may enhance positioning of the wire relative to the lens once the wire is deployed.

According to some embodiments, the handle 5500 may be equipped with an actuator that can be activated by a user to deploy and/or retract the wire 5330. According to some embodiments, the actuator of the handle 5500 may include a slider tab 5502, moveable by a user within a groove, for deployment (or “extension”) and retraction (or “withdrawal”) of wire 5330 within delivery shaft 5310. Slider tab 5502 may be coupled to one or more portions of the wire 5330 such that sliding the slider tab 5502 away from shaft 5310, in a proximal direction, pulls wire 5330 back into shaft 5410, while sliding slider tab 5502 towards shaft 5310, in a distal direction, pushes wire 5330 through the shaft 5410. As described above, sliding motion of the slider tab 5502 may also serve to expand or contract the wire loop like a lasso. Although a single slider tab 5302 is shown in FIGS. 55A-55C, according to some embodiments multiple sliders may be provided that slide in parallel within the groove to move ends of the wire 5330. Actuation of sliders such as slider tab 5502 can control, together or separately, ends of wire 5330 and/or other components to deploy, retract, and/or rotate the wires and/or components attached thereto. According to some embodiments, one or both ends of the wire 5330 may be coupled to slider tab 5502.

FIGS. 55B-55C shows a mechanism for manipulating the wire 5330 in more detail, according to some embodiments. FIG. 55B shows an isometric view of a partial cut-away of the extraction device 5300 with some components depicted with transparency to facilitate understanding. FIG. 55C shows a zoomed in side profile view of the partial cut-away from FIG. 55B.

As shown in FIGS. 55B-55C, the handle 5500 may include an outer shell that provides a housing for internal components of a slider mechanism. As shown in FIGS. 55B-55C, the slider tab 5502 may be operably coupled to the wire 5330 through inner shaft 5672. The inner shaft 5672 may be fixedly attached to portions of the wire 5330 and may have a cylindrical shape and/or other geometries. The inner shaft 5672 may also be fixedly attached to the slider tab 5502 such that sliding motion of the slider tab 5502 causes movement of the wire 5330 via sliding axial movement of the inner shaft 5672. According to some embodiments, the inner shaft 5672 may be supported by one or more support members 5676 disposed within the handle 5500. As shown in FIGS. 55B-55C, a distal portion of the handle 5500 may be coupled to the delivery shaft 5310. The handle 5500 may be fixedly coupled to the delivery shaft 5310 such that no relative movement between delivery shaft 5310 and handle 5500 occurs, allowing the wire to be extended and withdrawn through the delivery shaft 5310 based on movement of the slider tab 5502 relative to the handle 5500 and delivery shaft 5310. According to some embodiments, the distal portion of the handle 5500 may be coupled to the delivery shaft 5310 via a channel cylinder 5681 disposed within the handle housing. As shown in FIG. 55B-55C, the channel cylinder 5681 may be fixedly coupled to the delivery shaft and fixedly coupled to the handle 5500. The channel cylinder 5681 may also at least partially surround the inner shaft 5672, in an annular region within the handle 5500 and around the inner shaft 5672, such that the inner shaft 5672 slides back and forth through an inner channel of the channel cylinder 5681.

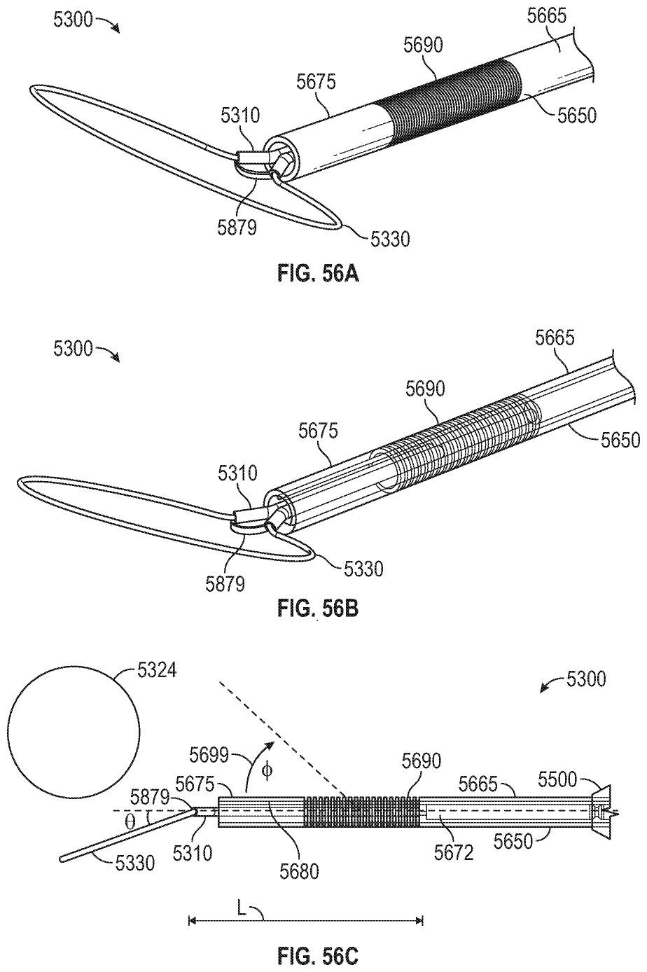

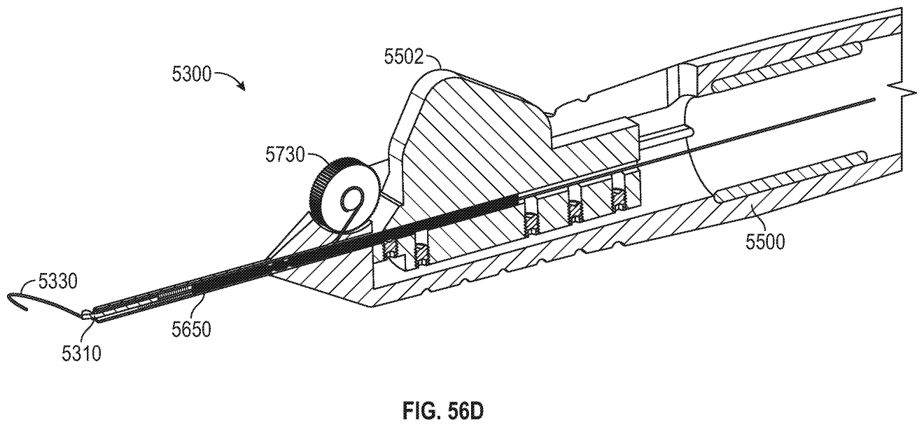

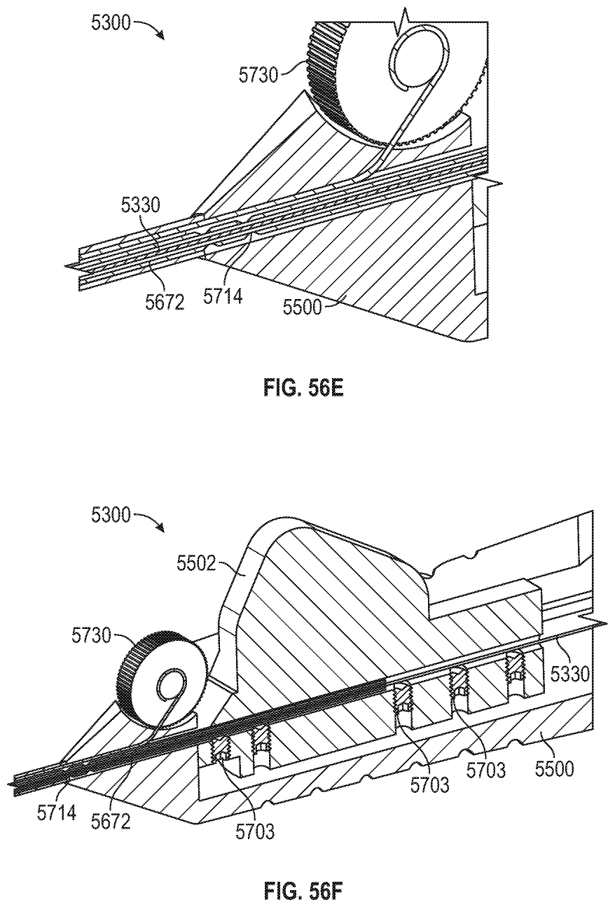

According to some embodiments, for example, as shown in FIG. 55D, the actuator of the handle 5500 may additionally or alternatively be equipped with a push button 5503. The actuator may be configured such that pressing the button 5503 deploys the wire 5330 and releasing the button retracts it. For example, the button 5503 may be biased in a released position (e.g., using a spring) and the button may be coupled to an inner shaft (e.g., inner shaft 5672) in a manner that converts depression of the button to axial motion of the shaft, thereby deploying (extending) the wire 5330 through the delivery shaft 5310. Although a single button 5503 is shown in FIG. 56C, according to some embodiments multiple buttons may be provided. Actuation of buttons such as button 5503 can control, together or separately, ends of wire 5330 and/or other components to deploy, retract, and/or rotate the wires and/or components attached thereto. According to some embodiments, one or both ends of the wire 5330 may be coupled to button 5503.

According to some embodiments, for example as shown in FIGS. 56A-56C, the extraction device 5300 can be equipped with an articulating arm 5650. The articulating arm 5650 can be configured to rotate a manipulator extending from the articulating arm 5650 to manipulate a lens or patient tissue. In various embodiments, the articulating arm can be configured to articulate in any direction, and the manipulator can include any one or more of the manipulator components described previously herein with respect to FIGS. 1-54), such as one or more wires, rings, covers, dissection tools, forceps, etc.

In some embodiments in which the manipulator includes a wire loop, the articulating arm 5650 can be configured to cause rotation of the wire 5330 in a direction transverse to a plane defined by the wire loop. Such movement can facilitate movement of the wire loop around a lens of a patent to, for example, facilitate subsequent cutting of the lens encircled by the wire loop via retraction of the wire. In various embodiments, the articulating arm can be configured to rotate the wire loop relative to the handle 5500 or another component proximal to the wire in any direction transverse to the loop.

According to some embodiments, for example as seen in FIGS. 56A-56C, the articulating arm 5650 can include a proximal segment 5665, a distal segment 5675, and an articulating joint 5690 between the proximal segment 5665 and the distal segment 5675. The articulating joint 5690 can rotatably connect the proximal segment 5665 to the distal segment 5675 so that rotation of the distal segment relative to the proximal segment can be used to move or rotate the wire 5330 or other manipulator extending from the distal segment 5675. The distal segment 5675 can be fixedly coupled to the delivery shaft 5310, so that the delivery shaft 5310 and a corresponding wire loop extending therefrom can move or rotate with the rotation of the distal segment 5675. The proximal segment 5665 can be fixedly coupled to the handle 5500 so that rotation of the distal segment 5675 relative to the proximal segment 5665 can rotate the distal segment 5675 relative to the handle 5500, and thereby cause rotation of the manipulator relative to the handle 5500.

The extraction device 5300 may employ an actuator for operating the articulating arm to cause the rotation of the distal segment 5675 relative to the proximal segment 5665. According to some embodiments, the actuator may include or be coupled to a pull cable 5680 that is operable to rotate the distal segment 5675 relative to the proximal segment 5665. The actuator may include a control component (such as a slider, roller, switch, button, etc.) on the handle 5500 that a user may manipulate to tension or pull on the pull cable 5680. A distal end portion of the pull cable can be coupled distal segment 5675 so that tension on the pull cable is converted to rotation of the distal segment. For example, the distal end portion of the pull cable can be welded or otherwise fixedly attached to the distal segment 5675. Although a single pull cable is shown, it is contemplated that other types of mechanisms may be employed to drive movement of the articulating arm, instead of or in combination with the pull cable. For example, the articulating arm may employ motors, linear actuators, bar linkages, cams, or other mechanisms for controlling motion of the articulating arm, or for converting movements of a control component on the handle to the desired rotational movement of the articulating arm. Further, it is contemplated that multiple pull cables may be used in some embodiments to drive rotation of the distal segment in different directions or to provide multiple degrees of freedom for the distal segment. Alternatively, or in combination with the actuator, in some embodiments the arm may be rotated about the articulating joint 5690 using preformed regions of the wire as the wire loop is deployed and extended through the wire. For example, the wire may have a preformed bent region that applies force to the articulating arm as the wire is extended therethrough, such that the wire urges rotation of the distal segment 5675 relative to the proximal segment 5665 about the articulating joint as the preformed bent region passes over the articulating joint.

The articulating arm 5650 may be configured with substantially any range of motion or degrees of freedom as desired. According to some embodiments, articulation of the articulating arm 5650 may be configured to cause bending rotation of the distal segment 5675 relative to the proximal segment 5665 about the joint 5690, such that a bend angle φ between a longitudinal axis of the distal segment 5675 and a longitudinal axis of the proximal segment 5665 changes with the rotation. Such a bending rotation may, for example, be used to move a wire loop extending from the distal segment down and around a lens of the eye.

According to some embodiments, the articulating arm 5650 may have a range of motion that permits the angle φ to be varied up to +/−180 degrees, where an angle φ of zero degrees corresponds to a straight arrangement in which the axis of the proximal segment 5665 and the distal segment 5675 are aligned with each other. According to some embodiments, the articulating arm may have a range of motion that permits the angle φ to be varied up to +/−90 degrees. According to some embodiments, the control component of the actuator is disposed on one side of the handle, and the articulating arm is configured to bend the distal segment 5675 toward the same side as the control component. According to other embodiments, the articulating arm is configured to bend the distal segment 5675 away from the side of the control component. According to some embodiments, the articulating arm is configured to bend the distal segment in either direction toward or away from the side of the control component as desired. According to some embodiments, the articulating arm 5650 may have a default or non-actuated position, in which the proximal segment 5665 and the distal segment 5675 are in a straight arrangement aligned with each other (angle φ=0), and an actuated position, in which the proximal and distal segments are bent relative to each other to form a non-zero angle φ. The control component can be biased to the non-actuated position such that the non-actuated position corresponds to a position of the articulating arm in the absence of any user interaction with a control component, while the actuated position corresponds to a position upon user interaction with the control component.

Alternatively, or in combination, articulation of the articulating arm 5650 may be configured to cause twisting rotation of the distal segment 5675 relative to the proximal segment 5665 about the joint 5690. In such embodiments, the distal segment can be configured to rotate about the longitudinal axis of the distal segment and/or the longitudinal axis of the proximal segment. In some embodiments employing a twisting rotation, the loop may extend to one side of the shaft, and the twisting rotation may be configured to swing the loop down and around a lens of the eye.

According to some embodiments, the range of motion of the articulating arm 5650 can be selected to facilitate interaction with a lens 5324. For example, and as further discussed herein, the manipulator can include a wire 5330 forming a wire loop when the wire 5330 is in a deployed position. The loop of the wire may define a plane, and as seen for example in FIG. 56C, the rotation of the articulating arm 5650 can be configured to move the wire loop in a direction that is transverse to the plane of the loop. For example, the distal segment 5675 can be fixedly coupled to the delivery shaft 5310, and articulation of the articulating arm can be configured to cause bending rotation in a direction shown by articulation arrow 5699.

According to some embodiments, the articulating arm 5650 can facilitate a procedure in which the arm is inserted into the eye, such as through a cornea and into the anterior chamber of the aye. The articulating arm 5650 can be configured to articulate in various ways depending on, for example, the anatomy of the eye or the preference of the surgeon operating the device. For example, the articulating arm 5650 may be inserted through a corneal incision with the bend angle φ of the articulating arm at a first angle. Next, the wire loop may be deployed with the bend angle φ of the articulating arm at a second angle. Then, the wire loop may be placed around the lens at a third angle. In various embodiments, each of the first, second, and third angle may be the same, or any two or more of the first, second, and third angles may be different than one another.

According to some embodiments in which the wire loop has a non-zero bend angle θ, as described previously, the articulating arm may be configured to rotate the distal segment 5675 in a direction opposite to the bend angle θ. An example of this motion is shown in FIG. 56C, in which the direction of the articulation arrow 5699 is opposite to the direction of the bend angle θ. Such motion can, for example, facilitate encircling the lens with the wire loop in cases where the delivery shaft approaches the lens through a corneal incision and through the anterior chamber. For example, the delivery shaft 5310 and the articulating arm 5650 may enter the eye through the corneal incision from above (anterior to) the native lens to be treated (e.g., the lens to be cut and/or removed). As the delivery shaft 5310 may form an angle with respect to the plane of the native lens, the bend angle of the wire loop allows the wire loop to be deployed from the shaft extended across the front side (anterior side) of the native lens, e.g., in a direction substantially parallel to the plane of the lens. Upon extension of the wire loop, the arm may then be articulated to rotate the wire loop down and around the lens, such that a portion of the wire loop is brought to posterior to the native lens encircling the lens.

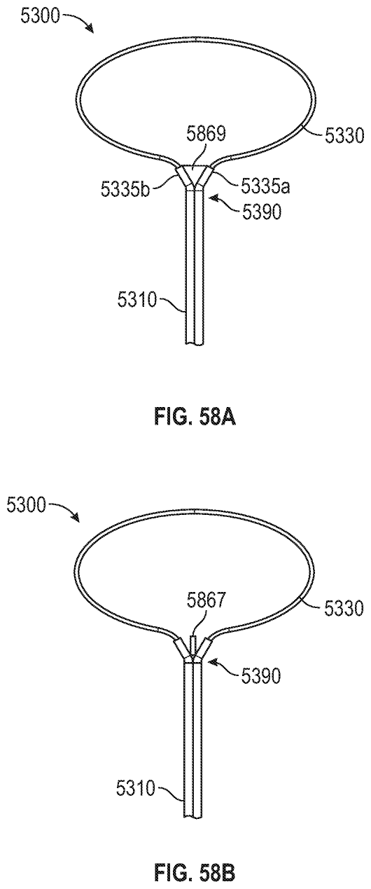

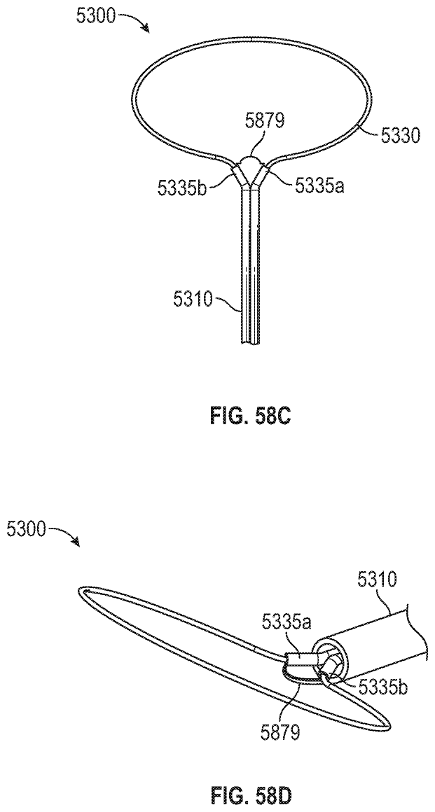

According to some embodiments, dimensions of the articulating arm 5650 may be selected to facilitate articulation of the arm while at least a portion of the arm is maintained within the eye. For example, the articulating arm 5650 may be dimensioned to permit rotation of the distal segment while the articulating joint 5690 is held within the eye, and while the arm is inserted through the eye through a small corneal or other eye incision. The articulating portion of the articulating arm may have a length L, which can be defined from the most proximal portion of the articulating joint to the distalmost end of the delivery shaft. As described further below with respect to FIGS. 58A-58C the distalmost end of the delivery shaft can provide a cutting face, such as a convex surface 5879. The wire loop may also have a diameter D, which can be a diameter of the wire loop measured along the same direction as the shaft axis 5361. In embodiments employing a circular wire loop, this diameter D can be the same when measured in any direction. In some embodiments of an oblong wire loop, this diameter D can be a shortest diameter of the wire loop corresponding to the minor axis, where the major axis of the wire loop is oriented perpendicular to the shaft axis. According to some embodiments, the length L may be approximately equal to the diameter D for a ratio of LID of 1. According to some embodiments, the length L may be greater than the diameter D, such as, for example, a ratio of L/D of 0.1. It is contemplated that the device may employ other dimensions for the ratio of L/D. According to some embodiments, the length L, or the combination of length L plus diameter D, may be no longer than a longest possible length from an inner surface of the cornea to an outer surface of the native lens. This longest length may vary from patient to patient, and the length L of the articulating portion of the articulating arm may vary depending on, for example, mode of operation or articulation direction. Such a configuration may enhance an ability for the surgeon to manipulate the articulating arm within the eye without creating an excessively large incision on the eye.

FIGS. 56D-56F are cutaway views illustrating components of a mechanism that can be used for actuation of the articulating arm. According to some embodiments, for example as seen in FIGS. 56D-56F, an actuator for operating the articulating arm may include a roller 5730. The roller 5730 may be operable to cause the rotation of the distal segment 5675 by tensioning the pull cable 5680. Rolling motion of the roller 5730 can increase or decrease tension on the pull cable 5680 so as to articulate the distal segment to cause bending rotation thereof about the joint 5690. The roller 5730 can be a wheel or other spinning member that rotates about an axis thereof to adjust tension on the pull cable.

According to some embodiments, the roller 5730 may be configured for direct interaction therewith by a user. For example, a surgeon may slide their thumb or other finger over the roller to spin the roller and thereby articulate the articulating arm 5650. According some embodiments, the roller 5730 may be configured to indirect interaction therewith by a user. For example, the roller 5730 may be coupled to another control component, such as a slider or a button, which is configured to interface with the roller 5730 to operate the roller for tensioning the pull cable 5680 or otherwise operating the articulating arm 5650. According to some embodiments, the roller 5730 may be configured for both of the aforementioned modes of operation depending on the user's preference (e.g., the surgeon may be able to operate the roller directly or indirectly, depending on their preference or the situation).

FIGS. 56D-56F also illustrate components of a mechanism that can be used for extension or retraction of a manipulator, such as wire 5330, through the articulating arm 5650. The example shown in FIGS. 56D-56F employs slider tab 5502, which can be configured as described previously, to extend and retract wire 5330 through a delivery shaft 5310 fixed to the distal segment of the articulating arm 5650.

The slider tab 5502 can be coupled to the wire 5330 in any suitable fashion. FIGS. 56E-56D show an example in which set screws 5703 and crimping locations 5672 are used to fixedly couple slider tab 5502, wire 5330, and inner shaft 5672 together so that the wire and inner shaft move together with movement of the slider tab. The set screws 5703 include a first set of set screws in a proximal portion of the slider 5502 to fix the wire 5330 to the slider, and a second set of set screws in a distal portion of the slider 5502 to fix the inner shaft 5672 to the slider. The inner shaft 5672 is further crimped against the wire 5330 at crimping locations 5714 to further fix the wire within the inner shaft for smooth engagement as the slider is actuated to extend and retract the wire via the inner shaft. It will be appreciated that any number of set screws may be used for either or both of the inner shaft and the wire. Further, any other suitable fastening mechanism may be used instead of or in addition to the set screws or crimping, such as adhesives, welding, etc.

According to some embodiments, the actuator for operating the articulating arm may be separate from the actuator for extension and retraction of the wire. According to some embodiments, a combined actuator may be used for both articulation of the articulating arm and for extension/retraction of the wire. For example a single lever or other control component may be operable for both rotation of the articulating arm and movement of the wire within the lumen of the delivery shaft.

FIGS. 56D-56F depict an example in which the actuator for rotating the articulating arm 5650 includes a roller 5730, and the actuator for extending and retracting the wire 5330 includes a slider tab 5502. According to some embodiments, the slider tab and the roller can be fully independently operable. Alternatively, or in combination, the slider tab 5502 may be configured to couple to the roller 5730 so that the operation of the slider is configured to articulate the arm in concert with the extension or retraction of the wire. For example, the slider tab 5502 may include a linear rack mated with the roller 5730 via a series of complementary mated gear teeth on the roller and the rack. Linear or axial sliding motion of the slider tab may thus be configured to rotate the roller via the mated teeth, so that the arm is articulated simultaneously with, after, or otherwise in concert with extension of the wire loop as the slider tab is operated by the user. It will be appreciated that other methods may be used to couple the slider tab and roller together, or to couple other control components together.

In the example shown, the articulating arm 5650 has a substantially tubular construction. Each of the proximal segment 5665 and the distal segment 5675 are constructed as tubular members that may be substantially rigid sections of the tubular arm. Internal components of the actuator mechanism such as the pull cable 5680 and inner shaft 5672 can extend through a lumen of the tubular articulating arm. The joint 5690 is a flexible tubular section between the proximal and distal segment that is bendable to provide a joint that permits articulation thereabout. Such a flexible tubular section may, for example, be constructed as a laser cut section of a nitinol tube or other metal tube that extends the length of the articulating arm, where the laser cut section forms a series of ribs (e.g., circumferential ribs) arranged along the length of the flexible section as seen for example in FIGS. 56A-56C. It is contemplated that the articulating arm can be constructed in numerous ways in various embodiments. For example the articulating arm or portions thereof may be constructed of metal, plastics, or other materials. The joint may be a flexible longitudinally extending segment, or may be configured as a hinge or fixed pivot point. The proximal and distal segments 5665 and 5675 can be non-tubular or non-hollow segments, and portions of the actuation mechanism such as the pull cable 5680 and/or the inner shaft 5672 may be positioned or routed external to segments of the articulating arm.

It is contemplated that various components coupled to each other can be separate components attached to each other or can be integrally formed as a unitary construction. For example, the delivery shaft 5310 can be a separate component that is fixedly attached to the distal segment 5675 of the articulating arm or can be an integral part thereof. Additionally or alternatively, the proximal segment 5665 can be a separate component that is fixedly attached to the handle 5500 of the extraction device 5300 or can be an integral part thereof. Additionally or alternatively, the proximal segment 5665, distal segment, 5675, and articulating joint 5690 can be separate components attached to each other or can be integrally formed with flexibility at the articulating joint 5690 to permit the desired articulation.

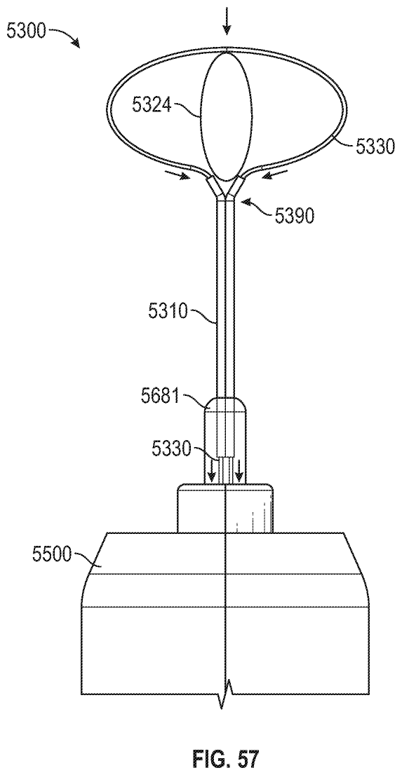

FIG. 57 shows a top view of extraction device 5300. According to some embodiments, for example, as shown in FIG. 57, at least a portion of the delivery shaft 5310 may include a generally Y-shaped hub 5390. The diverging channels 5335a and 5335b of the lumen may extend through corresponding first and second branching arms of the Y-shaped hub and form a Y-shaped lumen. The Y-shaped hub 5390 may also provide a support for the specimen when interacting with the specimen using the wire 5330. For example, as shown in FIG. 57, the Y-shaped hub 5390 may provide a crevice that can fixate the lens 5324 during operation, and allow the lens 5324 to be cut by retracting the wire 5330 while the lens 5324 is fixated or abutting against the Y-shaped hub 5390. According to some embodiments, the Y lumen may inhibit the lens from vaulting out of the lens bag by providing a counter-pressure during retraction and segmentation of the lens. FIG. 57 also shows an implementation of the extraction device 5300 where both ends of the wire 5330 are retracted simultaneously, which pulls the wire in the directions shown by arrows. According to some embodiments, both ends of the wire 5330 may be coupled to the slider tab 5502, such that retraction of the slider tab away from the delivery shaft 5310 pulls both ends of the wire in unison. This may further serve to prevent rotation of the lens 5324 during a cutting operation with the wire 5330.

According to some embodiments, for example as shown in FIG. 58A-58D, the extraction device 5300 can include a cutting face positioned between the channels. In such embodiments, the distal end of the delivery shaft 5310 may not have an empty crevice between the diverging channels due to the presence of the cutting face. The cutting face can be configured to press against the lens and contact the wire upon retraction of the wire so that the wire, once retracted, will rest against a distal part of the cutting face and fully transect the lenticular tissue. In some embodiments, without this cutting face the area between the channels may not allow for complete segmentation of the lens upon retraction of the wire. According to some embodiments, the cutting face includes a surface positioned distal to distal ends of the channels 5335a and 5335b to make a smooth transition for the wire at the exits of the channels when the wire is in a fully retracted position. For example, the distally positioned cutting face surface may make the bend or angle of the wire at the channel exits larger than a small bend radius or angle that would otherwise occur in the absence of the distally positioned cutting face surface. Such a smooth transition may serve to minimize damage or kinking of the wire.

According to some embodiments, for example as shown in FIG. 58A, the cutting face may include a transverse surface 5869. The transverse surface 5869 may be composed of a distal end or distalmost surface of a filler material between the two channels 5335a and 5335b. The transverse surface 5869 may extend across a region between the channels so that it spans the region between the two channels. FIG. 58A shows an example in which the transverse surface 5869 forms a flat surface for the cutting face.

According to some embodiments, for example, as shown in FIG. 58B, the distal end of the delivery shaft 5310 may alternatively have a projection 5867 protruding in a gap between the two channels to fully transect the material upon retraction of the wire 5330. In such embodiments, the cutting face may be formed from a surface at a distal end of the protrusion.

According to some embodiments, for example, as shown in FIG. 58C, the cutting face may include a convex surface 5879. As seen in FIG. 58C, the convex surface 5879 may be bowed outward distally so that it extends distally. The convex surface 5879 is shown extending distally beyond the distal ends of the channels 5335a and 5335b to facilitate a smooth transition for the wire in a fully retracted position, as described above. In various embodiments, the convex cutting face surface may have any appropriate convex shape. In some embodiments, and as seen in FIG. 58C, the convex cutting face surface may have a smooth arcuate surface to further facilitate a smooth transition with reduced kinking.

It is contemplated that the cutting face may be formed using any suitable construction between the channels at the distal end of the delivery shaft 5310, such as rigid plastics, metals, or the like. FIG. 58D shows an example construction of the cutting face configured with a convex surface like that shown in FIG. 58C, in which the cutting face and the channels 5335a and 5335b are formed from a bent tubular member having a cutaway region that can form the cutting face. In particular, the tubular member can include a first end portion forming the first channel 5335a, a second end portion forming the second channel 5335b, and a cutaway region between the first and second end portions. The tubular member can be bent or curved at the cutaway region, so that an exposed surface of the cutaway region forms the convex surface 5879 of the cutting face, extending distally and spanning all the way across the region between the first and second channels 5335a and 5335b.

According to some embodiments there may be notches on the inner surfaces of the branching arms of the hub 5390 to allow for more complete retraction of the wires into a common lumen proximal to where the two branching arms of the Y-lumen start.

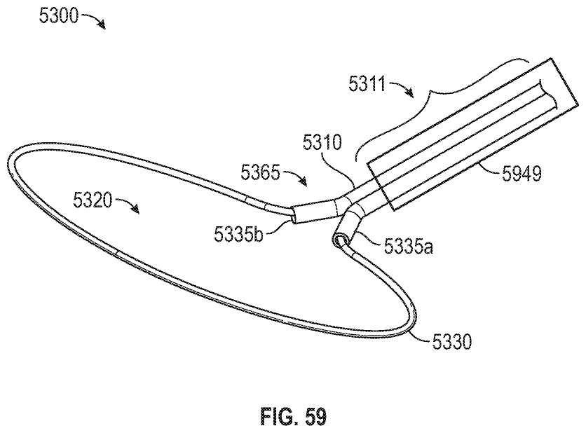

According to some embodiments, for example, as shown in FIG. 59, an outer sleeve 5949 may be disposed around the delivery shaft 5310. The outer sleeve may be configured to compress the branching arms 5365 of the delivery shaft 5310, thereby converging the channels accommodating the wire 5330 and causing the portions of the wire 5330 to retract into the branching arms 5365 at a different angle relative to the movement they exhibit when the branching arms 5365 are fully separated. The outer sleeve 5949 may be slidable in a distal direction relative to the delivery shaft 5310, for example, by retracting the branching arms or delivery shaft 5310 in a proximal direction into the sleeve 5949, or by pushing the sleeve 5949 in a distal direction over the branching arms or over the delivery shaft 5310.

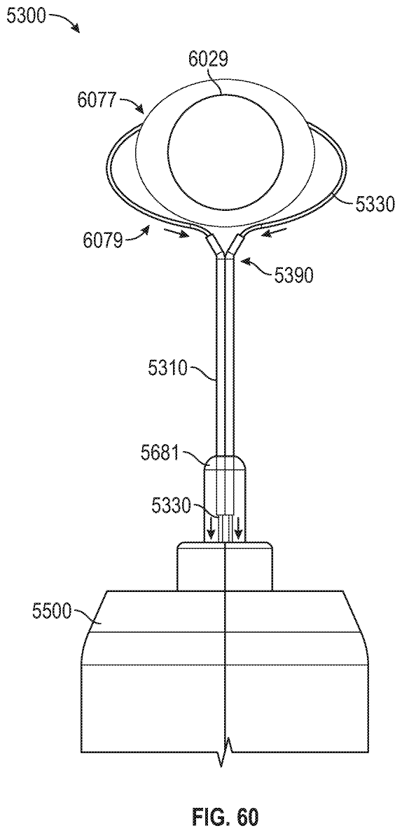

According to some embodiments, for example, as shown in FIG. 60, the design of the extraction device 5300 may allow for the wire 5330 to slide between an anterior leaflet of a capsulorhexis 6029, and position itself underneath the lens and above the posterior capsule. In this case, the wire 5330 may be introduced across the top of the lens, then loop the lens by slipping under the anterior lens capsule. The wire 5330 may then be retracted to cut the lens while the lens is still in the capsular bag. This procedure is illustrated in FIG. 60. As shown in FIG. 60, during this procedure a proximal portion 6079 of the wire 5330 may be positioned above the lens and outside of the capsulorhexis 6029. A distal portion 6077 of the wire 5330 may be positioned below the lens and above the posterior part of the lens capsule. The distal portion 6077 may slide under the capsulorhexis 6029 that is made in the capsular bag.

According to some embodiments, a method for using the extraction device 5300 includes making an incision (e.g., a corneal incision of less than 4 millimeters (mm)) to enter an anterior chamber of an eye of a patient. A capsulorhexis may be performed to form a capsulorhexis. Forming the capsulorhexis may remove a portion of a capsular bag of the eye. A lens 5324 of the eye may be hydrodissected and/or hydrodelineated so that the lens changes position from being entirely within the natural capsular bag to being partially or completely displaced from the capsular bag. Alternatively, this step may be omitted as the design of the extraction device 5300 may allow for the wire 5330 to be deployed between the lens and the capsular bag while the lens is still in the capsular bag. A viscoelastic may be used to further position the lens 5324 for optimum approach by the extraction device 5300.

After forming the capsulorhexis, a delivery shaft 5310 of extraction device 5300 may be inserted into the eye. The delivery shaft 5310 may be introduced to the anterior chamber of the eye through the incision and advanced forward. In some embodiments employing an extendable wire 5330, the wire may be in a proximal or retracted position during the insertion through the incision. In some embodiments employing an articulating arm 5650, the articulating arm may be in a straight or non-articulated configuration during the insertion through the incision. A wire loop of a wire 5330 may be advanced distally and expanded by extending the wire through either or both of channels 5335a and 5335b of a lumen of the delivery shaft using an actuator (e.g., slider tab 5502 or button 5503), to bring the wire to an extended and expanded configuration. The wire 5330 may be deployed between the lens and the capsular bag while the lens is still in the capsular bag. The wire loop may be lassoed around the lens 5324 so that it at least partially surrounds the lens 5324. In some embodiments employing an articulating arm 5650, the arm may be articulated during and/or after extension of the wire to move the wire loop down (in a posterior direction) and around the lens, so that the expanded wire loop encircles the lens. Articulation of the arm may be accomplished using an actuator (e.g., roller 5730). The arm may be articulated about the articulating joint 5690 while the articulating joint is maintained within the eye or within the anterior chamber. The lens may be fixated against a Y-shaped hub and/or cutting face of the delivery shaft 5310, such that the Y-shaped hub or cutting face contacts the lens. The wire loop may be retracted proximally using the actuator so that the lens is cut or segmented into pieces smaller than the original whole lens. To segment the lens, the lens may be dissected and/or bisected in situ (within the bag). The lens may be segmented by retracting the wire against the cutting face to cut tissue of the lens. In some embodiments, the actuator may retract both ends of the wire loop simultaneously to prevent rotation of the lens. The wire or encircling structure may or may not be advanced and retracted several times to further segment the lens. For example, after segmenting the lens in situ, the lens may be rotated using another instrument and the lens may be further segmented using a similar process.

After retracting the wire and cutting the lens, the delivery shaft 5310 may be removed from the anterior chamber of the eye. In some embodiments employing an extendable wire loop, the wire may be in a retracted positioned during the removal from the eye. In some embodiments employing an articulating arm 5650, the articulating arm may be in a straight or non-articulated configuration during the removal from the eye. A device such as a second suction device may be used to remove any remaining lens tissue. An intraocular lens (IOL) may be delivered into the lens capsule after removal of the cut lens tissue. The lens bag may be reformed with viscoelastic to receive an artificial intraocular lens for reversal of aphakia and refractive correction.

One or more features of any one of the extraction devices 100, 200, 300, 400, 600, 705, 805, 905, 1000, 1100, 1200, 1300, 1400, 1500, 1600, 1700, 1800, 2400, and 5300 can be combined with one or more features of any other one of the extraction devices 100, 200, 300, 400, 600, 705, 805, 905, 1000, 1100, 1200, 1300, 1400, 1500, 1600, 1700, 1800, 2400, and 5300. One or more features of any one of the covers and/or bags discussed herein can apply to any other one of the covers and/or bags and the deployment mechanisms such as guides associated therewith.

The subject technology is illustrated, for example, according to various aspects described above. Various examples of these aspects are described as numbered concepts or clauses (1, 2, 3, etc.) for convenience. These concepts or clauses are provided as examples and do not limit the subject technology. It is noted that any of the dependent concepts may be combined in any combination with each other or one or more other independent concepts, to form an independent concept. The following is a non-limiting summary of some concepts presented herein:

Concept 1. An extraction device, comprising:

a delivery shaft having a lumen and a distal end;

a first wire forming a first arc and being positionable distal to the distal end of the delivery shaft while ends of the first arc are at a distalmost end of the delivery shaft; and

a second wire forming a second arc and being positionable distal to the distal end of the delivery shaft while ends of the second arc are at the distalmost end of the delivery shaft,

wherein a distalmost extent of the first wire is distal to a distalmost extent of the second wire, wherein the first wire and the second wire are separately retractable relative to the delivery shaft.

Concept 2. The extraction device of Concept 1 or any other Concept, further comprising:

a first guide and a second guide moveable along the first wire toward the distalmost extent of the first wire from a first position to a second position more distal than the first position; and

a cover attached to the first guide and the second guide, wherein, while in the second position, the cover encompasses an outer cross-sectional dimension of each of (i) the first wire and (ii) the second wire.

Concept 3. The extraction device of Concept 1 or any other Concept, further comprising:

a first ring extending about the first wire and the second wire;

a first cover extending from the first ring to the delivery shaft;

a second ring extending about the first wire and the second wire; and

a second cover extending from the second ring to the distalmost end of the first wire.

Concept 4. An extraction device, comprising:

a delivery shaft having a lumen and a distal end;

a dissection tool distal to the distal end of the delivery shaft;

a first capture portion positionable on a first side of an axis of the delivery shaft and comprising a first cover; and

a second capture portion positionable on a second side of the axis, opposite the first side, and comprising a second cover,

wherein the first capture portion and the second capture portion are configured to move toward the axis upon actuation and define an enclosed space between the first cover and the second cover.

Concept 5. An extraction device, comprising:

a delivery shaft having a distal end;

an irrigation port at the distal end;

a loop having a fluid permeable cover; and

a blade being moveable from a retracted position to an actuated position across at least a portion of the loop.

Concept 6. An extraction device, comprising:

a delivery shaft having:

an inner cylindrical shaft structure with a distal end; and

an outer cylindrical shaft structure having a distal end with a sharp cutting edge, wherein the sharp cutting edge of the outer cylindrical shaft structure is deployable beyond the distal end of the inner cylindrical shaft structure.

Concept 7. An extraction device, comprising:

a delivery shaft having a distal end with a sharp cutting edge; and

a gripping apparatus that is extendible from within a lumen of the delivery shaft and operable to grip and pull tissue against the sharp cutting edge.

Concept 8. An extraction device, comprising:

a delivery shaft having a distal end; and

an excision member that is extendible from within a lumen of the delivery shaft, wherein the excision member comprises a control shaft and a plurality of layered cutting and encapsulation leaves.

Concept 9. The extraction device of Concept 8 or any other Concept, wherein, the layered cutting and encapsulation leaves are configured to be reticulated relative to each other about an axis to create an enclosure for a lens or portion thereof.

Concept 10. An extraction device, comprising:

a delivery shaft having a distal end;

a first frame extendible from the distal end of the delivery shaft and having a flexible bag structure attached thereto; and

a second frame extendible from the distal end of the delivery shaft and having a lid structure attached thereto, wherein, in an extended configuration for the first frame and the second frame, the flexible bag structure and the lid structure are configured to define and enclose a cavity within which at least a portion of a lens of a patient's eye is encapsulated.

Concept 11. The extraction device of Concept 10 or any other Concept, wherein the second frame is configured, upon extension from the distal end of the delivery shaft, to transect the encapsulated at least the portion of the lens of the patient's eye.

Concept 12. The extraction device of Concept 11 or any other Concept, further comprising one or more additional frames, each extendible from the distal end of the delivery shaft with the first frame and retractable from within the cavity into the distal end of the delivery shaft to dissect the at least the portion of the lens of the patient's eye held therein.

Concept 13. A method, comprising:

extending a first frame, having an attached flexible bag structure, and a second frame into an eye of a patient such that the flexible bag structure at least partially surrounds at least a portion of a lens of the eye of the patient;

extending a third frame having an attached lid structure along the first frame to encapsulate the at least the portion of the lens between the flexible bag structure and the lid structure; and

withdrawing the second frame to transect the encapsulated at least the portion of the lens.

Concept 14. The method of Concept 13 or any other Concept, further comprising, prior to extending the first frame with the attached flexible bag structure and the second frame into the eye of the patient, performing a hydrodissection and/or a hydrodelineation procedure to change a position of the lens from being entirely within a natural capsular bag of the lens to being partially or completely displaced from the natural capsular bag.

Concept 15. The method of Concept 13 or any other Concept, further comprising withdrawing the first frame and the third frame from the eye of the patient to remove the transected, encapsulated at least the portion of the lens from the eye.

Concept 16. An extraction device, comprising:

a delivery shaft having a distal end;

a first frame extendible from the distal end of the delivery shaft and having a flexible bag structure attached thereto, wherein the flexible bag structure includes a plurality of openings; and

a second frame extendible from the distal end of the delivery shaft and having a lid structure attached thereto, wherein, in an extended configuration for the first frame and the second frame, the flexible bag structure and the lid structure are configured to secure at least a portion of a lens of a patient's eye therebetween.

Concept 17. The extraction device of Concept 16 or any other Concept, wherein, upon withdrawal of the first frame and the second frame into the distal end of the delivery shaft while the at least the portion of the lens is secured between the flexible bag structure and the lid structure, the flexible bag structure is configured to strain the at least the portion of the lens through the plurality of openings.

Concept 18. The extraction device of Concept 16 or any other Concept, wherein the second frame is configured, upon extension from the distal end of the delivery shaft, to transect the lens.

Concept 19. The extraction device of Concept 16 or any other Concept, further comprising one or more additional frames, each extendible from the distal end of the delivery shaft with the first frame and retractable into the distal end of the delivery shaft to dissect the at least the portion of the lens of the patient's eye held therein.

Concept 20. The extraction device of Concept 16 or any other Concept, wherein the lid structure comprises a plurality of openings.

Concept 21. A method, comprising:

extending a first frame having an attached flexible bag structure with a plurality of openings from a distal end of a delivery shaft into an anterior chamber of an eye of a patient such that the flexible bag structure at least partially surrounds at least a portion of a lens of the eye of the patient;

extending a second frame having an attached lid structure along the first frame to secure the at least the portion of the lens between the flexible bag structure and the lid structure; and

withdrawing the first and second frames into the distal end of the delivery shaft to strain the at least the portion of the lens through the plurality of openings in the flexible bag structure into the anterior chamber.

Concept 22. The method of Concept 21 or any other Concept, further comprising:

extending a third frame into the eye of the patient together with the first frame; and

prior to withdrawing the first and second frames, withdrawing the third frame to transect the secured at least the portion of the lens.

Concept 23. The method of Concept 22 or any other Concept, further comprising suctioning the strained at least the portion of the lens from the anterior chamber.

Concept 24. An extraction device, comprising:

a delivery shaft having a lumen and a distal end; and

first, second, and third wire loops extendible from within the lumen at the distal end, wherein the first, second, and third wire loops are configured to separate upon extension from within the lumen to at least partially surround a lens of a patient's eye, and wherein the separated first, second, and third wire loops are configured to be withdrawn into the lumen to pass through and dissect the lens.

Concept 25. The extraction device of Concept 24 or any other Concept, further comprising:

a handle, wherein a proximal end of the delivery shaft is attached to the handle; and

a slider tab in the handle and attached to the first, second, and third wire loops, wherein the slider tab is slidable to extend and withdraw the first, second, and third wire loops.

Concept 26. The extraction device of Concept 24 or any other Concept, wherein the second wire loop comprises a middle wire loop disposed between the first and third loops, and wherein the first, second, and third wire loops are configured to linearly separate in a direction substantially perpendicular to a plane defined by the middle wire loop.

Concept 27. The extraction device of Concept 26 or any other Concept, further comprising a plurality of interconnecting wires that extend from the first wire loop to the middle wire loop and from the middle wire loop to the third wire loop.

Concept 28. The extraction device of Concept 24 or any other Concept, wherein the first, second, and third wire loops are configured to separate by a rotation of the second and third wire loops relative to the first wire loop.

Concept 29. The extraction device of Concept 28 or any other Concept, further comprising first, second, and third nested wire-guide structures within the delivery shaft and configured to control a rotational position of the first, second, and third wire loops.

Concept 30. The extraction device of Concept 29 or any other Concept, wherein the first wire-guide structure comprises:

a cylindrical main body; and

a plurality of protrusions on an outer surface of the cylindrical main body.

Concept 31. The extraction device of Concept 30 or any other Concept, wherein the second wire-guide structure comprises:

a cylindrical main body at least partially nested within the cylindrical main body of the first wire-guide structure; and

at least one slot for the second wire loop.

Concept 32. The extraction device of Concept 31 or any other Concept, wherein the third wire-guide structure comprises:

a cylindrical main body at least partially nested within the cylindrical main body of the second wire-guide structure; and

at least one slot for the third wire loop.

Concept 33. The extraction device of Concept 32 or any other Concept, wherein the second and third wire-guide structures are rotatable relative to the first wire-guide structure.

Concept 34. The extraction device of Concept 33 or any other Concept, wherein the first, second, and third wire-guide structures are slidable within the delivery shaft for extension and retraction of the first, second, and third wire loops.

Concept 35. An extraction device, comprising:

a delivery shaft having a lumen and a distal end;

first and second wire loops extendible from within the lumen at the distal end; and

a plurality of interconnecting wires that extend from the first wire loop to the second wire loop, wherein the first and second wire loops are configured to separate upon extension from within the lumen to at least partially surround a lens of a patient's eye, and wherein the separated first and second wire loops and the plurality of interconnecting wires are configured to be withdrawn into the lumen to pass through and dissect the lens.

Concept 36. The extraction device of Concept 35 or any other Concept, wherein the first and second wire loops are configured to linearly separate to a separated configuration in which the first wire loop is substantially parallel to the second wire loop.

Concept 37. The extraction device of Concept 35 or any other Concept, wherein the first and second wire loops are configured to separate by a rotation of the first or second wire loop.

Concept 38. The extraction device of Concept 37 or any other Concept, wherein the plurality of interconnecting wires comprises two relatively shorter interconnecting wires that extend between the first and second wire loops near a distal end of the loops and two relatively longer interconnecting wires that extend between the first and second wire loops nearer the distal end of the delivery shaft than the two relatively shorter interconnecting wires.

Concept 39. An extraction device, comprising:

a delivery shaft having a lumen and a distal end;

first, second, and third wire loops extendible from within the lumen at the distal end, wherein the first, second, and third wire loops are configured to separate upon extension from within the lumen and are maneuverable to at least partially surround a lens of a patient's eye;

a stent extendible from within the lumen around the extended first, second, and third wire loops; and

an encapsulation bag extendible from within the lumen around the extended stent and the extended first, second, and third wire loops.

Concept 40. The extraction device of Concept 39 or any other Concept, wherein the stent comprises an opening and the encapsulation bag comprises an opening configured to be coaligned with the opening in the stent in an extended configuration for the stent and the encapsulation bag.

Concept 41. The extraction device of Concept 39 or any other Concept, further comprising a spiral compaction wire configured to radially compress the encapsulation bag.

Concept 42. The extraction device of Concept 41 or any other Concept, wherein the first, second, and third wire loops are configured to be retracted into the lumen to pass through and cut the lens into pieces.

Concept 43. The extraction device of Concept 42 or any other Concept, wherein the stent is configured to be retracted into the lumen following retraction of the first, second, and third wire loops to cut the pieces into smaller pieces within the encapsulation bag.

Concept 44. The extraction device of Concept 43 or any other Concept, wherein the spiral compaction wire is configured to radially compress the encapsulation bag following retraction of the first, second, and third wire loops and the stent.

Concept 45. An extraction device, comprising:

a delivery shaft having a lumen and a distal end, wherein the lumen comprises a first channel and a second channel diverging at the distal end; and

a wire forming a wire loop and extending from the first channel and the second channel, wherein the wire is movable within the lumen.

Concept 46. The extraction device of Concept 45 or any other Concept, further comprising:

a handle coupled to the delivery shaft; and

an actuator coupled to the wire, wherein the actuator is operable to extend and withdraw the wire loop.

Concept 47. The extraction device of Concept 46 or any other Concept, wherein the actuator comprises a slider tab, wherein sliding motion of the slider tab relative to the handle is configured to extend and withdraw the wire loop.

Concept 48. The extraction device of Concept 46 or any other Concept, wherein the actuator comprises a push button, wherein depression of the push button is configured to extend the wire loop, and wherein release of the push button is configured to withdraw the wire loop.

Concept 49. The extraction device of Concept 46 or any other Concept, wherein a proximal end of the delivery shaft is fixedly coupled to a distal end of the handle.

Concept 50. The extraction device of Concept 46 or any other Concept, wherein the actuator is at least partially disposed in a groove of the handle.

Concept 51. The extraction device of Concept 46 or any other Concept, further comprising:

an inner shaft disposed in the handle, wherein the actuator is operatively coupled to the wire through the inner shaft.

Concept 52. The extraction device of Concept 45 or any other Concept, wherein the delivery shaft has a Y-shaped hub at the distal end.

Concept 53. The extraction device of Concept 52 or any other Concept, wherein the wire loop is configured to be retracted into the lumen to pass through a lens of a patient's eye and cut the lens into pieces while the lens is supported against the Y-shaped hub.

Concept 54. The extraction device of Concept 52 or any other Concept, further comprising:

a sleeve, disposed around the delivery shaft and slidable relative to the delivery shaft,

wherein the first channel extends through a first arm of the Y-shaped hub and the second channel extends through a second arm of the Y-shaped hub,

wherein motion of the sleeve in a distal direction relative to the delivery shaft is configured to compress the first arm and the second arm.

Concept 55. The extraction device of Concept 45 or any other Concept, wherein the loop of the wire has a non-zero bend angle relative to an axis of the delivery shaft.

Concept 56. The extraction device of Concept 55 or any other Concept, wherein the non-zero bend angle is in a range of 10-30 degrees relative to the axis of the delivery shaft.

Concept 57. The extraction device of Concept 45 or any other Concept, wherein the delivery shaft has a non-zero bend angle relative to an axis of a handle.

Concept 58. The extraction device of Concept 57 or any other Concept, wherein the non-zero bend angle is in a range of 10-30 degrees relative to the axis of the handle.

Concept 59. A method, comprising:

inserting a delivery shaft into an anterior chamber of an eye of a patient, the delivery shaft having a lumen and a distal end, the lumen having a first channel and a second channel diverging at the distal end; and

at least partially surrounding a lens of the eye with a wire forming a loop and extending from the a first channel and a second channel, wherein the wire is movable within the lumen.

Concept 60. The method of Concept 59 or any other Concept, further comprising:

cutting the lens into pieces by retracting the wire using an actuator of a handle, wherein the handle is coupled to the delivery shaft and the actuator is coupled to the wire.

Concept 61. The method of Concept 60 or any other Concept, further comprising:

supporting the lens against a Y-shaped hub while cutting the lens into pieces.

Concept 62. The method of Concept 60 or any other Concept, further comprising:

wherein cutting the lens into pieces is performed while the lens is still in a capsular bag.

Concept 63. The method of Concept 60 or any other Concept, further comprising:

forming a capsulorhexis in a capsular bag prior to inserting the delivery shaft; and

inserting a distal portion of the wire below the lens and above a posterior part of a capsular bag, wherein a proximal portion of the wire is disposed above the capsulorhexis when the distal portion of the wire is disposed below the lens.

Concept 64. An extraction device, comprising:

a delivery shaft having a lumen and a distal end, the lumen comprising a first channel and a second channel at the distal end;

a wire forming a wire loop and extending from the first channel and the second channel, the wire being extendable and retractable within the lumen; and

a cutting face positioned between the first channel and the second channel, the cutting face being configured to contact the wire upon retraction of the wire.

Concept 65. The extraction device of Concept 64 or any other Concept, wherein the cutting face comprises a surface positioned distal to a distal end of each of the first and second channels.

Concept 66. The extraction device of Concept 64 or any other Concept, wherein the cutting face comprises a convex surface extending distally and spanning across a region between the first and second channels.

Concept 67. The extraction device of Concept 66 or any other Concept, wherein the convex surface is a smooth arcuate surface.

Concept 68. The extraction device of Concept 66 or any other Concept, wherein the delivery shaft comprises a tubular member having a first end portion forming the first channel, a second end portion forming the second channel, and cutaway region between the first and second end portions that forms the cutting face.

Concept 69. The extraction device of Concept 64 or any other Concept, wherein the cutting face comprises a flat surface extending transverse to the first and second channels.

Concept 70. The extraction device of Concept 64 or any other Concept, wherein the cutting face comprises a surface at a distal end of a protrusion that extends through a gap between the first and second channels.

Concept 71. The extraction device of Concept 64 or any other Concept, further comprising:

a handle coupled to the delivery shaft; and

an actuator on the handle operable to retract the wire towards the cutting face.

Concept 72. The extraction device of Concept 71 or any other Concept, wherein the actuator comprises a push button, wherein depression of the push button is configured to extend the wire through the lumen, and wherein release of the push button is configured to retract the wire towards the cutting face.

Concept 73. The extraction device of Concept 64 or any other Concept, further comprising:

a handle coupled to the delivery shaft, wherein the distal end of the delivery shaft is rotatable relative to the handle about an articulating joint.

Concept 74. A method of cataract extraction, comprising:

inserting a delivery shaft of an extraction device into an eye of a patient;

extending a wire from the inserted delivery shaft to expand a loop of the wire, the wire being positioned in a pair of channels of the delivery shaft;

encircling a lens of the eye with the expanded loop of the wire;

contacting the encircled lens with a cutting face between the pair of channels; and

retracting the wire through the encircled lens and against the cutting face to cut tissue of the lens.

Concept 75. The method of Concept 74 or any other Concept, further comprising removing delivery shaft from the eye after retracting the wire, wherein the wire is in a retracted position during the inserting and the removing of the delivery shaft.

Concept 76. The method of Concept 74 or any other Concept, further comprising:

prior to inserting the delivery shaft, forming a capsulorhexis to remove a portion of a capsular bag of the eye;

after cutting the tissue of the lens, removing the cut tissue from the eye; and

after removing the cut tissue, delivering an intraocular lens into the capsular bag.

Concept 77. The method of Concept 74 or any other Concept, wherein the lens is encircled by rotating the delivery shaft about an articulating joint of the extraction device to move the expanded loop around the lens.

Concept 78. An extraction device, comprising:

a handle;

an articulating arm coupled to the handle, the articulating arm having a distal segment, a proximal segment, and an articulating joint between the distal segment and the proximal segment;

a manipulator configured to extend from the distal segment of the articulating arm to manipulate a lens of an eye; and

an actuator on the handle operable to rotate of the distal segment relative to the proximal segment about the articulating joint.

Concept 79. The extraction device of Concept 78 or any other Concept, wherein the manipulator comprises a wire configured to extend from the distal segment to form a loop, and to retract into the distal segment to cut a lens encircled by the loop.

Concept 80. The extraction device of Concept 79 or any other Concept, wherein rotation of the distal segment relative to the proximal segment about the articulating joint is configured to move the loop in a direction transverse to a plane defined by the loop.

Concept 81. The extraction device of Concept 79 or any other Concept, wherein the distal segment is fixed to a pair of channels that each accommodate a portion of the wire, and wherein the proximal segment is fixed to the handle.

Concept 82. The extraction device of Concept 81 or any other Concept, further comprising a cutting face positioned between the pair of channels, wherein the cutting face is configured to contact the wire upon retraction of the wire.

Concept 83. The extraction device of Concept 78 or any other Concept, wherein the actuator is a first actuator, and the extraction device further comprises a second actuator on the handle separate from the first actuator, wherein the second actuator is operable to extend and retract the manipulator from the distal segment of the articulating arm.

Concept 84. The extraction device of Concept 78 or any other Concept, wherein the actuator is a combined actuator configured to extend and retract the manipulator within the distal segment of the articulating arm in concert with rotation of the distal segment.

Concept 85. The extraction device of Concept 78 or any other Concept, wherein the actuator includes a roller operable to rotate the distal segment, wherein the extraction device further comprises a slider operable to extend and retract the manipulator within the distal segment of the articulating arm.

Concept 86. The extraction device of Concept 85 or any other Concept, wherein the slider is coupled to the roller so that operation of the slider is further configured to operate the roller to rotate the distal segment.

Concept 87. The extraction device of Concept 78 or any other Concept, further comprising:

a pull cable having a first end portion fixedly coupled to the distal segment, wherein operation of the actuator is configured to pull on the pull cable to rotate the distal segment.

Concept 88. The extraction device of Concept 78 or any other Concept, wherein the articulating arm comprises a bendable tubular member in which the articulating joint is formed of a flexible portion of the bendable tubular member.

Concept 89. The extraction device of Concept 88 or any other Concept, wherein the bendable tubular member is a nitinol tube, and the flexible portion is a laser cut segment of the nitinol tube, the laser cut segment forming a series of ribs.

Concept 90. The extraction device of Concept 78 or any other Concept, wherein the actuator is operable from a non-actuated state, in which an axis of the distal segment and an axis of the proximal segment are aligned with each other, to an actuated state, in which the axis of the distal segment and the axis of the proximal segment form a non-zero angle relative to each other.

Concept 91. The extraction device of Concept 78 or any other Concept, wherein the actuator comprises a control component on a side of the handle, and the actuator is configured to rotate the distal segment in a direction towards the control component.

Concept 92. The extraction device of Concept 78 or any other Concept, wherein the actuator comprises a control component on a side of the handle, and the actuator is configured to rotate the distal segment in a direction away from the control component.

Concept 93. An extraction device, comprising:

an articulating arm having a distal segment, a proximal segment, and an articulating joint between the distal segment and the proximal segment;

a wire configured to extend out of the distal segment of the articulating arm to form a loop around lens of an eye; and

an actuator configured to operate the articulating arm to rotate the distal segment relative to the proximal segment about the articulating joint.

Concept 94. The extraction device of Concept 93 or any other Concept, wherein the rotation of the distal segment relative to the proximal segment is configured to move the loop in a direction transverse to a plane defined by the loop.

Concept 95. A method of cataract extraction, comprising:

inserting an articulating arm of a device into an eye of a patient;

rotating a distal segment of the articulating arm relative to a proximal segment of the articulating arm about a joint therebetween to move a wire extending out from the distal segment around a lens of the eye; and

while the wire is around the lens, retracting the wire to cut the lens.

Concept 96. The method of Concept 95 or any other Concept, wherein the articulating arm is in a straight configuration during the inserting the articulating arm, wherein the rotating of the distal segment moves the articulating arm from a straight configuration to a bent configuration.

Concept 97. The method of Concept 96 or any other Concept, further comprising:

returning the articulating arm to the straight configuration; and

removing the articulating arm from the eye with the articulating arm in the straight configuration.

Concept 98. The method of Concept 95 or any other Concept, further comprising:

extending the wire through the articulating arm to expand a loop of the wire, wherein the rotating of the distal segment moves the wire in a direction transverse to a plane of the loop.

Concept 99. The method of Concept 95 or any other Concept, wherein the joint is positioned within the eye during the rotating of the distal segment.

Concept 100. The method of Concept 95 or any other Concept, wherein the rotating of the distal segment and the retracting of the wire are each performed by operating an actuator in a handle coupled to the proximal segment.

Concept 101. The method of Concept 95 or any other Concept, wherein the distal segment is rotated by operating a roller to tension a pull cable coupled to the distal segment.

Concept 102. An extraction device, comprising:

a delivery shaft defining a shaft axis, the delivery shaft having a lumen and a distal end; and

a wire movable within the lumen between a retracted position and an extended position, wherein in the extended position the wire forms an oblong loop having a longitudinal dimension and a lateral dimension greater than the longitudinal dimensional, and wherein the wire has a natural shape that is expanded relative to the loop so that the delivery shaft applies a force to the wire to constrain the wire in the loop when in the extended position.

Concept 103. The extraction device of Concept 102 or any other Concept, wherein the natural shape of the wire is an open configuration.

Concept 104. The extraction device of Concept 102 or any other Concept, wherein the natural shape of the wire has preformed curved regions that urge the loop into a lenticular shape when the wire is in the extended configuration.

Concept 105. The extraction device of Concept 10 or any other Concept 2, wherein the wire loop has a first region of higher curvature than a second region, and the wire has a smaller cross-sectional area at the first region than the second region.

Concept 106. The extraction device of Concept 102 or any other Concept, further comprising a cutting face configured to contact a portion of the wire when the wire is in the retracted position, wherein the portion has a sharper cross-sectional shape than another portion of the wire.

Concept 107. The extraction device of Concept 102 or any other Concept, further comprising a cutting face configured to contact a portion of the wire when the wire is in the retracted position, wherein the portion has a reduced cross-sectional area than another portion of the wire.

Concept 108. The extraction device of Concept 102 or any other Concept, wherein the wire comprises a segment having a flat cross-section and another segment having a round cross-section.

Concept 109. The extraction device of Concept 108 or any other Concept, wherein the segment having a flat cross-section is configured to minimize out of plane bending of the wire.

Concept 110. The extraction device of Concept 109 or any other Concept, wherein the flat section has a long axis oriented in a direction transverse to a plane defined by the loop.

Concept 111. The extraction device of Concept 102 or any other Concept, wherein, when in the extended position, an exit region of the wire located proximal to the delivery shaft has a different cross-sectional shape than a distal region of the wire.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

A phrase such as “an aspect” does not imply that such aspect is essential to the subject technology or that such aspect applies to all configurations of the subject technology. A disclosure relating to an aspect may apply to all configurations, or one or more configurations. An aspect may provide one or more examples of the disclosure. A phrase such as “an aspect” may refer to one or more aspects and vice versa. A phrase such as “an embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples of the disclosure. A phrase such “an embodiment” may refer to one or more embodiments and vice versa. A phrase such as “a configuration” does not imply that such configuration is essential to the subject technology or that such configuration applies to all configurations of the subject technology. A disclosure relating to a configuration may apply to all configurations, or one or more configurations. A configuration may provide one or more examples of the disclosure. A phrase such as “a configuration” may refer to one or more configurations and vice versa.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Terms such as “top,” “bottom,” “front,” “rear” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Furthermore, to the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

While certain aspects and embodiments of the subject technology have been described, these have been presented by way of example only, and are not intended to limit the scope of the subject technology. Indeed, the novel methods and systems described herein may be embodied in a variety of other forms without departing from the spirit thereof. The accompanying claims and their equivalents are intended to cover such forms or modifications as would fall within the scope and spirit of the subject technology.

1. An extraction device, comprising:
a delivery shaft having a lumen and a distal end, the lumen comprising a first channel and a second channel at the distal end;
a wire forming a wire loop and extending from the first channel and the second channel, the wire being extendable and retractable within the lumen; and
a cutting face positioned between the first channel and the second channel, the cutting face being configured to contact the wire upon retraction of the wire.

2. The extraction device of claim 1, wherein the cutting face comprises a surface positioned distal to a distal end of each of the first and second channels.

3. The extraction device of claim 1, wherein the cutting face comprises a convex surface extending distally and spanning across a region between the first and second channels.

4. The extraction device of claim 3, wherein the convex surface is a smooth arcuate surface.

5. The extraction device of claim 3, wherein the delivery shaft comprises a tubular member having a first end portion forming the first channel, a second end portion forming the second channel, and cutaway region between the first and second end portions that forms the cutting face.

6. The extraction device of claim 1, wherein the cutting face comprises a flat surface extending transverse to the first and second channels.

7. The extraction device of claim 1, wherein the cutting face comprises a surface at a distal end of a protrusion that extends through a gap between the first and second channels.

8. The extraction device of claim 1, further comprising:
a handle coupled to the delivery shaft; and
an actuator on the handle operable to retract the wire towards the cutting face.

9. The extraction device of claim 8, wherein the actuator comprises a push button, wherein depression of the push button is configured to extend the wire through the lumen, and wherein release of the push button is configured to retract the wire towards the cutting face.

10. The extraction device of claim 1, further comprising:
a handle coupled to the delivery shaft, wherein the distal end of the delivery shaft is rotatable relative to the handle about an articulating joint.

11. A method of cataract extraction, comprising:
inserting a delivery shaft of an extraction device into an eye of a patient;
extending a wire from the inserted delivery shaft to expand a loop of the wire, the wire being positioned in a pair of channels of the delivery shaft;
encircling a lens of the eye with the expanded loop of the wire;
contacting the encircled lens with a cutting face between the pair of channels; and
retracting the wire through the encircled lens and against the cutting face to cut tissue of the lens.

12. The method of claim 11, further comprising removing delivery shaft from the eye after retracting the wire, wherein the wire is in a retracted position during the inserting and the removing of the delivery shaft.

13. The method of claim 11, further comprising:
prior to inserting the delivery shaft, forming a capsulorhexis to remove a portion of a capsular bag of the eye;
after cutting the tissue of the lens, removing the cut tissue from the eye; and
after removing the cut tissue, delivering an intraocular lens into the capsular bag.

14. The method of claim 11, wherein the lens is encircled by rotating the delivery shaft about an articulating joint of the extraction device to move the expanded loop around the lens.

15. An extraction device, comprising:
a handle;
an articulating arm coupled to the handle, the articulating arm having a distal segment, a proximal segment, and an articulating joint between the distal segment and the proximal segment;
a manipulator configured to extend from the distal segment of the articulating arm to manipulate a lens of an eye; and
an actuator on the handle operable to rotate of the distal segment relative to the proximal segment about the articulating joint.

16. The extraction device of claim 15, wherein the manipulator comprises a wire configured to extend from the distal segment to form a loop, and to retract into the distal segment to cut a lens encircled by the loop.

17. The extraction device of claim 16, wherein rotation of the distal segment relative to the proximal segment about the articulating joint is configured to move the loop in a direction transverse to a plane defined by the loop.

18. The extraction device of claim 16, wherein the distal segment is fixed to a pair of channels that each accommodate a portion of the wire, and wherein the proximal segment is fixed to the handle.

19. The extraction device of claim 18, further comprising a cutting face positioned between the pair of channels, wherein the cutting face is configured to contact the wire upon retraction of the wire.

20. The extraction device of claim 15, wherein the actuator is a first actuator, and the extraction device further comprises a second actuator on the handle separate from the first actuator, wherein the second actuator is operable to extend and retract the manipulator from the distal segment of the articulating arm.

21-67. (canceled)
